SEQUENCE LISTING (89 sequences)

SEQ ID NO: 1 - protein, 455 aa, Artificial Sequence
  Synthetic Sequence (PD-1/HC)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, Artificial Sequence
  Synthetic Sequence (PD-1/LC)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 455 aa, Artificial Sequence
  Synthetic Sequence (PD-1/HC, S131C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 455 aa, Artificial Sequence
  Synthetic Sequence (PD-1/HC, L128C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 455 aa, Artificial Sequence
  Synthetic Sequence (PD-1/HC, A129C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLCPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 455 aa, Artificial Sequence
  Synthetic Sequence (PD-1/HC, F170C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 455 aa, Artificial Sequence
  Synthetic Sequence (PD-1/HC, A141C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGTACLGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, Artificial Sequence
  Synthetic Sequence (PD-1/LC, P119C)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPSVFIFCPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 9 - protein, 219 aa, Artificial Sequence
  Synthetic Sequence (PD-1/LC, S121C)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 10 - protein, 219 aa, Artificial Sequence
  Synthetic Sequence (PD-1/LC, T164C)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVCEQDSKDSTYSLSSTLTLSKADYEKHKVYACEVTHQGLSSPVTKSFNRGEA

SEQ ID NO: 11 - protein, 219 aa, Artificial Sequence
  Synthetic Sequence (PD-1/LC, L135C)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCCLNNFYPREAKVQWKVDNALQSGNSQESVTEQDSKDSTYSLSSTLTLSKADYEKHKVYACEVTHQGLSSPVTKSFNRGEA

SEQ ID NO: 12 - protein, 455 aa, Artificial Sequence
  Synthetic Sequence (PD-1/HC)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 219 aa, Artificial Sequence; identical to SEQ ID NO: 2
  Synthetic Sequence (PD-1/LC)

SEQ ID NO: 14 - protein, 448 aa, Artificial Sequence
  Synthetic Sequence (CTLA-4/HC)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 215 aa, Artificial Sequence
  Synthetic Sequence (CTLA-4/LC)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 448 aa, Artificial Sequence
  Synthetic Sequence (CTLA-4/HC, F126C)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 215 aa, Artificial Sequence
  Synthetic Sequence (CTLA-4/LC, S121C)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 18 - protein, 448 aa, Artificial Sequence
  Synthetic Sequence (CTLA-4/HC, L128C)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 448 aa, Artificial Sequence
  Synthetic Sequence (CTLA-4/HC, F170C)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 215 aa, Artificial Sequence
  Synthetic Sequence (CTLA-4/LC, T164C)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVCEQDSKDSTYSLSSTLTLSKADYEKHKVYACEVTHQGLSSPVTKSFNRGEA

SEQ ID NO: 21 - protein, 448 aa, Artificial Sequence
  Synthetic Sequence (CTLA-4/HC, S131C)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 215 aa, Artificial Sequence
  Synthetic Sequence (CTLA-4/LC, P119C)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFCPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 23 - protein, 455 aa, Artificial Sequence
  Synthetic Sequence (PD-1/HC, F126C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 455 aa, Artificial Sequence
  Synthetic Sequence (PD-1/HC, L128C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 455 aa, Artificial Sequence
  Synthetic Sequence (PD-1/HC, F170C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 455 aa, Artificial Sequence
  Synthetic Sequence (PD-1/HC, S131C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, Artificial Sequence
  Synthetic Sequence (CTLA-4/LC)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 448 aa, Artificial Sequence
  Synthetic Sequence (CTLA-4/HC, P171C)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 214 aa, Artificial Sequence
  Synthetic Sequence (CTLA-4/LC, S165C)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGQPKAAPSVTLFPPSSEELQANKATLVCLISDFYPGAVTVAWKADSSPVKAGVETTTPCKQSNNKYAASSYLSLTPEQWKSHRSYSCQVTHEGSTVEKTVAPTEAS

SEQ ID NO: 30 - protein, 218 aa, Artificial Sequence
  Synthetic Sequence (PD-1/LC)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 455 aa, Artificial Sequence
  Synthetic Sequence (PD-1/HC, P171C)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 218 aa, Artificial Sequence
  Synthetic Sequence (PD-1/LC, S165C)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKGQPKAAPSVTLFPPSSEELQANKATLVCLISDFYPGAVTVAWKADSSPVKAGVETTTPCKQSNNKYAASSYLSLTPEQWKSHRSYSCQVTHEGSTVEKTVAPTEAS

SEQ ID NO: 33 - protein, 455 aa, Artificial Sequence
  PD-1/HC, T139R, F170C
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGRAALGCLVKDYFPEPVTVSWNSGALTSGVHTCPAVLQSSGLYSLSSVVTVPSSSLGTQTYICNVNHKPSNTKVDKKVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 219 aa, Artificial Sequence
  Synthetic Sequence (PD-1/LC, S114E, T164C)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPEVFIFPPSDEQLKSGTASVVCLLNNFYPREAKVQWKVDNALQSGNSQESVCEQDSKDSTYSLSSTLTLSKADYEKHKVYACEVTHQGLSSPVTKSFNRGEA

SEQ ID NO: 35 - protein, 448 aa, Artificial Sequence
  CTLA-4/HC, T139D
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGDAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 215 aa, Artificial Sequence
  Synthetic Sequence (CTLA-4/LC, S114K)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPKVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 455 aa, Artificial Sequence
  Synthetic Sequence (PD-1/HC)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGRAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 219 aa, Artificial Sequence
  Synthetic Sequence (PD-1/LC)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPEVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 455 aa, Artificial Sequence
  Synthetic Sequence (PD-1/HC)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSSASTKGPSVFPLAPSSKSTSGGDAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 219 aa, Artificial Sequence
  Synthetic Sequence (PD-1/LC)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIKRTVAAPKVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 448 aa, Artificial Sequence
  Synthetic Sequence (CTLA-4/HC)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGRAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - protein, 215 aa, Artificial Sequence
  Synthetic Sequence (CTLA-4/LC)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPEVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 5 aa, Artificial Sequence
  Synthetic Sequence (PD-1/HCDR1)
DYEMH

SEQ ID NO: 44 - protein, 17 aa, Artificial Sequence
  Synthetic Sequence (PD-1/HCDR2)
LIDPETGGTVYNQKFKD

SEQ ID NO: 45 - protein, 15 aa, Artificial Sequence
  Synthetic Sequence (PD-1/HCDR3)
ERFSYYGSTSDWYFD

SEQ ID NO: 46 - protein, 16 aa, Artificial Sequence
  Synthetic Sequence (PD-1/LCDR1)
RSSQSLVHSTGNTYLE

SEQ ID NO: 47 - protein, 7 aa, Artificial Sequence
  Synthetic Sequence (PD-1/LCDR2)
KVSNRFS

SEQ ID NO: 48 - protein, 9 aa, Artificial Sequence
  Synthetic Sequence (PD-1/LCDR3)
FQGSHVPYT

SEQ ID NO: 49 - protein, 125 aa, Artificial Sequence
  Synthetic Sequence (PD-1/VH)
EVQLVQSGAEVKKPGSSVKVSCKASGGTFSDYEMHWVRQAPGQGLEWMGLIDPETGGTVYNQKFKDRVTITADKSTSTAYMELSSLRSEDTAVYYCARERFSYYGSTSDWYFDVWGQGTTVTVSS

SEQ ID NO: 50 - protein, 112 aa, Artificial Sequence
  Synthetic Sequence (PD-1/VL)
DIVMTQTPLSLPVTPGEPASISCRSSQSLVHSTGNTYLEWYLQKPGQSPQLLIYKVSNRFSGVPDRFSGSGSGTDFTLKISRVEAEDVGVYYCFQGSHVPYTFGGGTKVEIK

SEQ ID NO: 51 - protein, 5 aa, Artificial Sequence
  Synthetic Sequence (CTLA-4/HCDR1)
SYTMH

SEQ ID NO: 52 - protein, 17 aa, Artificial Sequence
  Synthetic Sequence (CTLA-4/HCDR2)
FISYDGNNKYYADSVKG

SEQ ID NO: 53 - protein, 9 aa, Artificial Sequence
  Synthetic Sequence (CTLA-4/HCDR3)
TGWLGPFDY

SEQ ID NO: 54 - protein, 12 aa, Artificial Sequence
  Synthetic Sequence (CTLA-4/LCDR1)
RASQSVGSSYLA

SEQ ID NO: 55 - protein, 7 aa, Artificial Sequence
  Synthetic Sequence (CTLA-4/LCDR2)
GAFSRAT

SEQ ID NO: 56 - protein, 9 aa, Artificial Sequence
  Synthetic Sequence (CTLA-4/LCDR3)
QQYGSSPWT

SEQ ID NO: 57 - protein, 118 aa, Artificial Sequence
  Synthetic Sequence (CTLA-4/VH)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 58 - protein, 108 aa, Artificial Sequence
  Synthetic Sequence (CTLA-4/VL)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 59 - protein, 445 aa, Artificial Sequence
  Synthetic Sequence (J591/HC)
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 213 aa, Artificial Sequence
  Synthetic Sequence (J591/LC)
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 61 - protein, 453 aa, Artificial Sequence
  Synthetic Sequence (006/HC, F170C)
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 214 aa, Artificial Sequence
  Synthetic Sequence (006/LC, T164C)
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVCEQDSKDSTYSLSSTLTLSKADYEKHKVYACEVTHQGLSSPVTKSFNRGEA

SEQ ID NO: 63 - protein, 445 aa, Artificial Sequence; identical to SEQ ID NO: 59
  Synthetic Sequence (J591/HC)

SEQ ID NO: 64 - protein, 214 aa, Artificial Sequence
  Synthetic Sequence (J591/LC)
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 453 aa, Artificial Sequence
  Synthetic Sequence (006/HC, P171C)
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - protein, 213 aa, Artificial Sequence
  Synthetic Sequence (006/LC, S165C)
DIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKGQPKAAPSVTLFPPSSEELQANKATLVCLISDFYPGAVTVAWKADSSPVKAGVETTTPCKQSNNKYAASSYLSLTPEQWKSHRSYSCQVTHEGSTVEKTVAPTEAS

SEQ ID NO: 67 - protein, 689 aa, Artificial Sequence
  Synthetic Sequence (CD40-Fc-FAP/HC, P171C)
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLQQSGVEVKKPGASVTVSCRASGYSFADHFIHWVRQAPGQGFQWMGWINPNRGVTHHAQDFQGRVAMTRDMSTDTVYMELTSLRSDDTAVYYCARDASLTARPYYFYGFDVWGQGTLVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSS

SEQ ID NO: 68 - protein, 219 aa, Artificial Sequence
  Synthetic Sequence (CD40/LC)
DIVMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 213 aa, Artificial Sequence
  Synthetic Sequence (FAP/LC, S165C)
DIQMTQSPSSVSASVGDRVTITCRASQGISSWLAWYQQKPGKAPKLLIYAASSLQSGVPSRFSGSGSGTDFTLTISSLQPEDFATYYCQQANSFPPAFGQGTKVEIKGQPKAAPSVTLFPPSSEELQANKATLVCLISDFYPGAVTVAWKADSSPVKAGVETTTPCKQSNNKYAASSYLSLTPEQWKSHRSYSCQVTHEGSTVEKTVAPTESS

SEQ ID NO: 70 - protein, 689 aa, Artificial Sequence
  Synthetic Sequence (CD40-Fc-FAP/HC, P171C)
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSSASTKGPSVFPLAPSSKSTSGGTAALGCLVKDYFPEPVTVSWNSGALTSGVHTFCAVLQSSGLYSLSSVVTVPSSSLGTQTYICNVNHKPSNTKVDKKVEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLQQSGVEVKKPGASVTVSCRASGYSFADHFIHWVRQAPGQGFQWMGWINPNRGVTHHAQDFQGRVAMTRDMSTDTVYMELTSLRSDDTAVYYCARDASLTARPYYFYGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 71 - protein, 218 aa, Artificial Sequence
  Synthetic Sequence (CD40/LC, S165C)
DIVMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIKGQPKAAPSVTLFPPSSEELQANKATLVCLISDFYPGAVTVAWKADSSPVKAGVETTTPCKQSNNKYAASSYLSLTPEQWKSHRSYSCQVTHEGSTVEKTVAPTESS

SEQ ID NO: 72 - protein, 214 aa, Artificial Sequence
  Synthetic Sequence (FAP/LC)
DIQMTQSPSSVSASVGDRVTITCRASQGISSWLAWYQQKPGKAPKLLIYAASSLQSGVPSRFSGSGSGTDFTLTISSLQPEDFATYYCQQANSFPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 5 aa, Artificial Sequence
  Synthetic Sequence (CD40/HCDR1 )
TYWIT

SEQ ID NO: 74 - protein, 17 aa, Artificial Sequence
  Synthetic Sequence (CD40/HCDR2)
DIHPGSGSTKYNEKFKS

SEQ ID NO: 75 - protein, 16 aa, Artificial Sequence
  Synthetic Sequence (CD40/LCDR1)
RSSQNIVNSQGNTYLE

SEQ ID NO: 76 - protein, 7 aa, Artificial Sequence
  Synthetic Sequence (CD40/LCDR2)
KVTNRFS

SEQ ID NO: 77 - protein, 9 aa, Artificial Sequence
  Synthetic Sequence (CD40/LCDR3)
FQASLVPWT

SEQ ID NO: 78 - protein, 112 aa, Artificial Sequence
  Synthetic Sequence (CD40/VH)
QVQLVQSGAEVKKPGASVKVSCKASGYILTTYWITWVRQAPGQGLEWMGDIHPGSGSTKYNEKFKSRVTLTVDTSISTAYMELSRLRSEDTAVYYCARRDYWGQGTTVTVSS

SEQ ID NO: 79 - protein, 112 aa, Artificial Sequence
  Synthetic Sequence (CD40/VL)
DIVMTQSPLSLPVTPGEPASISCRSSQNIVNSQGNTYLEWYLQKPGQSPQLLIYKVTNRFSGVPDRFSGSGSGTDFTLKISRVEAEDVGVYYCFQASLVPWTFGGGTKVEIK

SEQ ID NO: 80 - protein, 5 aa, Artificial Sequence
  Synthetic Sequence (FAP/HCDR1)
DHFIH

SEQ ID NO: 81 - protein, 17 aa, Artificial Sequence
  Synthetic Sequence (FAP/HCDR2)
WINPNRGVTHHAQDFQG

SEQ ID NO: 82 - protein, 16 aa, Artificial Sequence
  Synthetic Sequence (FAP/HCDR3)
DASLTARPYYFYGFDV

SEQ ID NO: 83 - protein, 11 aa, Artificial Sequence
  Synthetic Sequence (FAP/LCDR1)
RASQGISSWLA

SEQ ID NO: 84 - protein, 7 aa, Artificial Sequence
  Synthetic Sequence (FAP/LCDR2)
AASSLQS

SEQ ID NO: 85 - protein, 9 aa, Artificial Sequence
  Synthetic Sequence (FAP/LCDR3)
QQANSFPPA

SEQ ID NO: 86 - protein, 125 aa, Artificial Sequence
  Synthetic Sequence (FAP/VH)
QVQLQQSGVEVKKPGASVTVSCRASGYSFADHFIHWVRQAPGQGFQWMGWINPNRGVTHHAQDFQGRVAMTRDMSTDTVYMELTSLRSDDTAVYYCARDASLTARPYYFYGFDVWGQGTLVTVSS

SEQ ID NO: 87 - protein, 107 aa, Artificial Sequence
  Synthetic Sequence (FAP/VL)
DIQMTQSPSSVSASVGDRVTITCRASQGISSWLAWYQQKPGKAPKLLIYAASSLQSGVPSRFSGSGSGTDFTLTISSLQPEDFATYYCQQANSFPPAFGQGTKVEIK

SEQ ID NO: 88 - protein, 98 aa, Artificial Sequence
  Synthetic Sequence (CH1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 89 - protein, 107 aa, Artificial Sequence [sequence omitted]

(19) United States
(12) Patent Application Publication
(10) Pub. No.: US 2024/0010754 A1
(43) Pub. Date: Jan. 11, 2024

(54) MULTISPECIFIC ANTIGEN BINDING PROTEIN

(71) Applicant: JIANGSU HENGRUI MEDICINE CO., LTD., Jiangsu (CN)

(72) Inventors: Wei ZHANG, Jiangsu (CN); Fuwei JIANG, Jiangsu (CN); Leilei WANG, Jiangsu (CN); Simeng CHEN, Jiangsu (CN); Cheng LIAO, Jiangsu (CN)

(21) Appl. No.: 18/265,217

(22) PCT Filed: Dec. 3, 2021
(86) PCT No.: PCT/CN2021/135254

§ 371 Date: Jun. 2, 2023

(30) Foreign Application Priority Data
Dec. 3, 2020 (CN) 202011412507.X

(51) Int. Cl. C07K 16/46; C07K 16/28

(52) U.S. Cl. CPC C07K 16/468; C07K 16/2818; C07K 2317/94; C07K 2317/522; C07K 2317/72; C07K 2317/31; C07K 2317/71

(57) ABSTRACT

Provided is a multispecific antigen binding protein. Provided is a multispecific antigen binding protein comprising one or more amino acid substitutions at CH1 and CL, a composition comprising same, a preparation method therefor, and a medical use thereof. The specific antigen binding protein effectively reduces the mismatching of light chains.

### TECHNICAL FIELD

The present disclosure belongs to the field of biopharmaceutics and particularly relates to a multispecific antigen-binding protein, a preparation method therefor and use thereof in pharmaceutics.

### BACKGROUND

Being capable of recognizing different antigen molecules or different epitopes of the same antigen molecule, bispecific antibodies have distinctive biological functions that monoclonal antibodies do not have and are gradually accepted by the market. Although the technology related to bispecific antibodies has developed for twenty years, there remain many realistic technical problems that restrict the production and development of bispecific antibodies. As technology has advanced, many novel molecular formats and strategies for modifying and producing bispecific antibodies have emerged. Take 1+1 asymmetric (Fab A+Fab B) bispecific antibodies as an example: to avoid light chain mispairing (a light chain for antigen A being paired with a heavy chain for antigen B, or a light chain for antigen B being paired with a heavy chain for antigen A), a number of strategies have been reported as yet.

Common light chain antibodies: It has been reported that in vitro display technologies or mice with common light chains were used to select specific light chains (WO2012067176; WO2013134263), so as to pair a heavy chain for antigen A with a heavy chain for antigen B and maintain the original biological functions of the corresponding antibodies. Two-in-one antibodies: It has been reported that phage display and rational design (WO2010027981) were used to optimize an antibody that binds to antigen A so that it had the ability to bind to antigen B while retaining its original ability to bind to antigen A, and thus one antibody binding to two targets was achieved. Requiring a large amount of engineering, both the strategies described above are technically difficult to implement; their universality remains to be confirmed. Therefore, the engineering of Fab with orthogonality (VH/VL or/and CH1/CL interaction interface) has received increasing attention in the industry in recent years.

IgG/TCR (WO2014014796; WO2019057122): It has been reported that the CH1/CL of FabA was replaced with the TCR constant region to avoid potential light chain mispairing in view of the structural similarity between the antibody CH1/CL and the TCR constant region. Crossmab (WO2012023053): VH/VL, CH1/CL or HC/LC for a Fab were interchanged to reduce the possibility of light chain mispairing. DuetMab (WO2013096291): The original disulfide bond in the CH1/CL of a Fab for antigen A was replaced with a non-natural disulfide bond to reduce the possibility of light chain mispairing. Computer-aided design: Computer-aided design (WO2014150973; WO2016172485) was used to avoid light chain mispairing.

As a novel drug format, bispecific antibodies have special structures, and their preparation and industrialization are therefore more difficult than those of monoclonal antibodies. Although there have been many approaches attempting to address the mispairing problem between a heavy chain and a light chain, the consequent structural adjustments may alter the stability, immunogenicity or pharmacokinetic properties of the molecule. There remains a need to develop new techniques to improve the yields of multispecific antibodies (e.g., bispecific antibodies).

### SUMMARY

The present disclosure increases the proportion of correct pairing of a light chain and a heavy chain in multispecific antibodies relative to wild types by removing a natural disulfide bond from the CH1/CL interface and introducing a non-natural disulfide bond into the interface, or by introducing a pair of electrostatically complementary amino acids into the CH1/CL interface, or by removing a natural disulfide bond from the CH1/CL interface and introducing a non-natural disulfide bond and also a pair of electrostatically complementary amino acids into the interface.

The present disclosure provides a dimerized polypeptide comprising a heavy chain constant region 1 (CH1) and a light chain constant region (CL), wherein: CH1 and CL comprise natural-non-cysteine-to-cysteine amino acid substitutions at positions selected from one or more of (i-1) to (i-6):
(i-1) position 170 of CH1 and position 164 of CL,
(i-2) position 128 of CH1 and position 121 of CL,
(i-3) position 129 of CH1 and position 121 of CL,
(i-4) position 131 of CH1 and position 119 of CL,
(i-5) position 141 of CH1 and position 135 of CL, and
(i-6) position 171 of CH1 and position 165 of CL.

In the context of the present disclosure, heavy chain positions are numbered according to the EU numbering scheme; for example, the positions of the amino acid substitutions in CH1 are numbered on the basis of the CH1 (SEQ ID NO: 88) of human IgG1; light chain positions are numbered according to the Kabat numbering scheme; for example, the positions of the amino acid substitutions in CL are numbered on the basis of the human κ light chain (IGLC, SEQ ID NO: 89).

(SEQ ID NO: 88)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
(SEQ ID NO: 89)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

It will be appreciated by those skilled in the art that other IgG subtypes other than IgG1, such as IgG2, IgG3 and IgG4, comprising, at counterparts of the positions at which IgG1 CH1 comprises the amino acid mutations of the present disclosure, identical types of amino acid mutations also fall within the protection scope of the present disclosure.

In some embodiments, a natural disulfide bond may or may not be included between CH1 and CL.

In some embodiments, CH1 retains the natural cysteine at position 220, and CL retains the natural cysteine at position 214.

In some embodiments, the natural cysteine at position 220 of CH1 and/or the natural cysteine at position 214 of CL are/is substituted with an amino acid other than cysteine.

In some embodiments, CH1 comprises an amino acid substitution C220A, and CL comprises an amino acid substitution C214A.

In some embodiments, CH1 and CL comprise the following amino acid substitutions:
(a) C220A in CH1 and C214A in CL; and
(b) amino acid substitutions selected from at least one of the following:
   (b-1) F170C in CH1 and T164C in CL;
   (b-2) L128C in CH1 and S121C in CL;
   (b-3) A129C in CH1 and S121C in CL;
   (b-4) S131C in CH1 and P119C in CL;
   (b-5) A141C in CH1 and L135C in CL; and
   (b-6) P171C in CH1 and S165C in CL.

In some embodiments, CH1 and CL comprise the following amino acid substitutions: (a) C220A in CH1 and C214A in CL; and (b) F170C in CH1 and T164C in CL.

In some embodiments, CH1 and CL comprise the following amino acid substitutions: (a) C220A in CH1 and C214A in CL; and (b) P171C in CH1 and S165C in CL.

In some embodiments, CH1 and CL comprise amino acid substitutions that cause an electrostatic interaction interface to be formed between CH1 and CL.

In some embodiments, the amino acid substitutions that cause an electrostatic interaction interface to be formed between CH1 and CL are at position 139 of CH1 and position 114 of CL.

In some embodiments, the amino acid at position 139 of CH1 is substituted with a positively charged amino acid, and the amino acid at position 114 of CL is substituted with a negatively charged amino acid; or the amino acid at position 139 of CH1 is substituted with a negatively charged amino acid, and the amino acid at position 114 of CL is substituted with a positively charged amino acid.

In some embodiments, the positively charged amino acid is selected from the group consisting of K, R and H; the negatively charged amino acid is selected from the group consisting of D and E.

In some embodiments, CH1 and CL comprise amino acid substitutions selected from any one of the following:
(1) T139R in CH1 and S114E in CL;
(2) T139R in CH1 and S114D in CL;
(3) T139K in CH1 and S114E in CL;
(4) T139K in CH1 and S114D in CL;
(5) T139D in CH1 and S114K in CL;
(6) T139D in CH1 and S114R in CL;
(7) T139E in CH1 and S114K in CL; and
(8) T139E in CH1 and S114R in CL.

In some embodiments, CH1 and CL comprise the following amino acid substitutions:
(a) C220A in CH1 and C214A in CL; and
(b) amino acid substitutions selected from at least one of the following:
   (b-1) F170C in CH1 and T164C in CL;
   (b-2) L128C in CH1 and S121C in CL;
   (b-3) A129C in CH1 and S121C in CL;
   (b-4) S131C in CH1 and P119C in CL;
   (b-5) A141C in CH1 and L135C in CL; and
   (b-6) P171C in CH1 and S165C in CL; and
(c) amino acid substitutions selected from any one of the following:
   (c-1) T139R in CH1 and S114E in CL;
   (c-2) T139R in CH1 and S114D in CL;
   (c-3) T139K in CH1 and S114E in CL; and
   (c-4) T139K in CH1 and S114D in CL.

In some embodiments, CH1 and CL comprise the following amino acid substitutions:
(a) C220A in CH1 and C214A in CL;
(b) amino acid substitutions selected from at least one of the following:
   (b-1) F170C in CH1 and T164C in CL;
   (b-2) L128C in CH1 and S121C in CL;
   (b-3) A129C in CH1 and S121C in CL;
   (b-4) S131C in CH1 and P119C in CL;
   (b-5) A141C in CH1 and L135C in CL; and
   (b-6) P171C in CH1 and S165C in CL; and
(c) amino acid substitutions selected from any one of the following:
   (c-1) T139D in CH1 and S114K in CL;
   (c-2) T139D in CH1 and S114R in CL;
   (c-3) T139E in CH1 and S114K in CL; and
   (c-4) T139E in CH1 and S114R in CL.

In some embodiments, CH1 and CL comprise the following amino acid substitutions: (a) C220A in CH1 and C214A in CL; (b) F170C in CH1 and T164C in CL; and (c) T139R in CH1 and S114E in CL.

In some embodiments, CH1 and CL comprise the following amino acid substitutions: (a) C220A in CH1 and C214A in CL; (b) F170C in CH1 and T164C in CL; and (c) T139D in CH1 and S114K in CL.

In some embodiments, CH1 and CL comprise the following amino acid substitutions: (a) C220A and C214A; (b) P171C in CH1 and S165C in CL; and (c) T139R in CH1 and S114E in CL.

In some embodiments, CH1 and CL comprise the following amino acid substitutions: (a) C220A in CH1 and C214A in CL; (b) P171C in CH1 and S165C in CL; and (c) T139D in CH1 and S114K in CL.

In some embodiments, CL is from an antibody λ light chain (Cλ) or κ light chain (Cκ).

The present disclosure provides an antigen-binding protein comprising the dimerized polypeptide described above.

In some embodiments, the antigen-binding protein comprises a first antigen-binding domain, and the first antigen-binding domain comprises a Fab comprising a first heavy chain variable region VH1, a first light chain variable region VL1, and the dimerized polypeptide; in the dimerized polypeptide, the CH1 is a first CH1, and the CL is a first CL; VH1 and the first CH1 are linked directly or by a linker, and VL1 and the first CL are linked directly or by a linker. In some embodiments, the C-terminus of VH1 and the N-terminus of the first CH1 are linked directly or by a linker, and the C-terminus of VL1 and the N-terminus of the first CL are linked directly or by a linker.

In some embodiments, the linker is a peptide linker. In some embodiments, the peptide linker is a peptide with an amino acid sequence of at least 5 amino acids; in one embodiment, the peptide linker is a peptide with an amino acid sequence of 5 to 100 amino acids; in a further embodiment, the peptide linker is a peptide with an amino acid sequence of 10 to 50 amino acids. In one embodiment, the peptide linker is (GxS)n or (GxS)nGm, wherein G=glycine, S=serine, and (x=3, n=3, 4, 5 or 6, and m=0, 1, 2 or 3) or (x=4, n=2, 3, 4 or 5, and m=0, 1, 2 or 3). In one embodiment, x=4 and n=3 or 4. In one embodiment, the peptide linker is (G4S)4.

In some embodiments, the antigen-binding protein comprises a first antigen-binding domain and a second antigen-binding domain, wherein the second antigen-binding domain comprises a second heavy chain variable region VH2 and a second light chain variable region VL2, and the first antigen-binding domain and the second antigen-binding domain bind to different antigens or bind to different epitopes on the same antigen; in some embodiments, the second antigen-binding domain comprises a Fab. The Fab comprises a second heavy chain variable region VH2, a second heavy chain constant region 1 (a second CH1), a second light chain variable region VL2, and a second light chain constant region (a second CL2). In some embodiments, the C-terminus of VH2 and the N-terminus of the second CH1 are linked directly or by a linker, and the C-terminus of VL2 and the N-terminus of the second CL are linked directly or by a linker.

In some embodiments, the second CH1 and the second CL do not comprise natural-non-cysteine-to-cysteine amino acid substitutions selected from one or more of the following:
(i-1) position 170 of the second CH1 and position 164 of the second CL,
(i-2) position 128 of the second CH1 and position 121 of the second CL,
(i-3) position 129 of the second CH1 and position 121 of the second CL,
(i-4) position 131 of the second CH1 and position 119 of the second CL,
(i-5) position 141 of the second CH1 and position 135 of the second CL, and
(i-6) position 171 of the second CH1 and position 165 of the second CL.

In the context of the present disclosure, heavy chain positions are numbered according to the EU numbering scheme; for example, the positions of the amino acid substitutions in CH1 are numbered on the basis of the CH1 (SEQ ID NO: 88) of human IgG1; light chain positions are numbered according to the Kabat numbering scheme; for example, the positions of the amino acid substitutions in CL are numbered on the basis of the human κ light chain (IGLC, SEQ ID NO: 89).

(SEQ ID NO: 88)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
(SEQ ID NO: 89)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

It will be appreciated by those skilled in the art that other IgG subtypes other than IgG1, such as IgG2, IgG3 and IgG4, comprising, at counterparts of the positions at which IgG1 CH1 comprises the amino acid mutations of the present disclosure, identical types of amino acid mutations also fall within the protection scope of the present disclosure.

In some embodiments, the second CH1 and the second CL do not comprise natural-non-cysteine-to-cysteine amino acid substitutions.

In some embodiments, the second CH1 and the second CL retain the natural cysteines 220C and 214C.

In some embodiments, the second CH1 and the second CL do not comprise natural-non-cysteine-to-cysteine amino acid substitutions and retain the natural cysteines 220C and 214C.

In some embodiments, the first CH1 and the first CL comprise the following amino acid substitutions:
(a) C220A in CH1 and C214A in CL; and
(b) amino acid substitutions selected from at least one of the following:
   (b-1) F170C in CH1 and T164C in CL;
   (b-2) L128C in CH1 and S121C in CL;
   (b-3)A129C in CH1 and S121C in CL;
   (b-4) S131C in CH1 and P119C in CL;
   (b-5) A141C in CH1 and L135C in CL; and
   (b-6) P171C in CH1 and S165C in CL;
      and the second CH1 and the second CL do not comprise natural-non-cysteine-to-cysteine amino acid substitutions and retain the natural cysteines 220C and 214C.

In some embodiments, the first CH1 and the first CL comprise the following amino acid substitutions:
(a) C220A in CH1 and C214A in CL; and
(b) F170C in CH1 and T164C in CL;
and the second CH1 and the second CL do not comprise natural-non-cysteine-to-cysteine amino acid substitutions and retain the natural cysteines 220C and 214C.

In some embodiments, the first CH1 and the first CL comprise the following amino acid substitutions: (a) C220A in CH1 and C214A in CL; and (b) P171C in CH1 and S165C in CL; and the second CH1 and the second CL do not comprise natural-non-cysteine-to-cysteine amino acid substitutions and retain the natural cysteines 220C and 214C.

In some embodiments, the first CH1 and the first CL comprise amino acid substitutions that cause an electrostatic interaction interface to be formed between the first CH1 and the first CL; and/or the second CH1 and the second CL comprise amino acid substitutions that cause an electrostatic interaction interface to be formed between the second CH1 and the second CL.

In some embodiments, the amino acids for forming an electrostatic interaction interface in the first CH1 and the second CH1 are oppositely charged, and the amino acids for forming an electrostatic interaction interface in the first CL and the second CL are oppositely charged.

In some embodiments, the amino acid substitutions that cause an electrostatic interaction interface to be formed between the first CH1 and the first CL are at position 139 of the first CH1 and position 114 of the first CL; and/or the amino acid substitutions that cause an electrostatic interaction interface to be formed between the second CH1 and the second CL are at position 139 of the second CH1 and position 114 of the second CL.

In some embodiments, position 139 of the first CH1 and position 139 of the second CH1 are each substituted with an oppositely charged amino acid, and position 114 of the first CL and position 114 of the second CL are each substituted with an oppositely charged amino acid.

In some embodiments, the amino acid at position 139 of the first CH1 is substituted with a positively charged amino acid, and the amino acid at position 114 of the first CL is substituted with a negatively charged amino acid; or the amino acid at position 139 of the first CH1 is substituted with a negatively charged amino acid, and the amino acid at position 114 of the first CL is substituted with a positively charged amino acid; and/or

the amino acid at position 139 of the second CH1 is substituted with a negatively charged amino acid, and the amino acid at position 114 of the second CL is substituted with a positively charged amino acid; or the amino acid at position 139 of the second CH1 is substituted with a positively charged amino acid, and the amino acid at position 114 of the second CL is substituted with a negatively charged amino acid.
In some embodiments, the positively charged amino acid is selected from the group consisting of K, R and H; the negatively charged amino acid is selected from the group consisting of D and E.

In some embodiments, the first CH1 and the first CL comprise amino acid substitutions selected from any one of the following:

(1) T139R in CH1 and S114E in CL; T139R in CH1 and S114D in CL; T139K in CH1 and S114E in CL; T139K in CH1 and S114D in CL; T139D in CH1 and S114K in CL; T139D in CH1 and S114R in CL; T139E in CH1 and S114K in CL; and T139E in CH1 and S114R in CL; and/or
the second CH1 and the second CL comprise amino acid substitutions selected from the group consisting of: T139R in CH1 and S114E in CL; T139R in CH1 and S114D in CL; T139K in CH1 and S114E in CL; T139K in CH1 and S114D in CL; T139D in CH1 and S114K in CL; T139D in CH1 and S114R in CL; T139E in CH1 and S114K in CL; and T139E in CH1 and S114R in CL.

In some embodiments, the first CH1 and the first CL comprise amino acid substitutions selected from the group consisting of: T139R in CH1 and S114E in CL; T139R in CH1 and S114D in CL; T139K in CH1 and S114E in CL; and T139K in CHI and S114D in CL; and/or

the second CH1 and the second CL comprise amino acid substitutions selected from the group consisting of: T139D in CH1 and S114K in CL; T139D in CH1 and S114R in CL; T139E in CH1 and S114K in CL; and T139E in CH1 and S114R in CL.

In some embodiments, the first CH1 and the first CL comprise amino acid substitutions selected from the group consisting of: T139D in CH1 and S114K in CL; T139D in CH1 and S114R in CL; T139E in CH1 and S114K in CL; and T139E in CH1 and S114R in CL; and/or
the second CH1 and the second CL comprise amino acid substitutions selected from the group consisting of: T139R in CH1 and S114E in CL; T139R in CH1 and S114D in CL; T139Kin CH1 and S114E in CL; and T139Kin CH1 and S114D in CL.

In some embodiments, the first CH1 and the first CL comprise the following amino acid substitutions:
(a) C220A in CH1 and C214A in CL;
(b) amino acid substitutions selected from at least one of the following: F170C in CH1 and T164C in CL; L128C in CH1 and S121C in CL; A129C in CH1 and S121C in CL; S131C in CH1 and P119C in CL; A141C in CH1 and L135C in CL; and P171C in CH1 and S165C in CL; and
(c) amino acid substitutions selected from one of the following: T139R in CH1 and S114E in CL; T139R in CH1 and S114D in CL; T139K in CH1 and S114E in CL; and T139K in CH1 and S114D in CL;
   and the second CH1 and the second CL comprise amino acid substitutions selected from the group consisting of: T139D in CH1 and S114K in CL; T139D in CH1 and S114R in CL; T139E in CH1 and S114K in CL; and T139E in CH1 and S114R in CL.

In some embodiments, the first CH1 and the first CL comprise the following amino acid substitutions:
(a) C220A in CH1 and C214A in CL;
(b) amino acid substitutions selected from at least one of the following: F170C in CH1 and T164C in CL; L128C in CH1 and S121C in CL; A129C in CH1 and S121C in CL; S131C in CH1 and P119C in CL; A141C in CH1 and L135C in CL; and P171C in CH1 and S165C in CL; and
(c) amino acid substitutions selected from one of the following: T139D in CH1 and S114K in CL; T139D in CH1 and S114R in CL; T139E in CH1 and S114K in CL; and T139E in CH1 and S114R in CL;
   and the second CH1 and the second CL comprise amino acid substitutions selected from the group consisting of: T139R in CH1 and S114E in CL; T139R in CH1 and S114D in CL; T139Kin CH1 and S114E in CL; and T139Kin CH1 and S114D in CL.

In some embodiments, the first CH1 and the first CL comprise the following amino acid substitutions:
(a) C220A in CH1 and C214A in CL;
(b) F170C in CH1 and T164C in CL; and
(c) T139R in CH1 and S114E in CL;
   and the second CH1 and the second CL comprise the following amino acid substitutions: T139D in CH1 and S114K in CL.

In some embodiments, the first CH1 and the first CL comprise the following amino acid substitutions:
(a) C220A in CH1 and C214A in CL;
(b) F170C in CH1 and T164C in CL; and
(c) T139D in CH1 and S114K in CL;
   and the second CH1 and the second CL comprise the following amino acid substitutions: T139R in CH1 and S114E in CL.

In some embodiments, the first CH1 and the first CL comprise the following amino acid substitutions:
(a) C220A in CH1 and C214A in CL;
(b) P171C in CH1 and S165C in CL; and
(c) T139R in CH1 and S114E in CL;
   and the second CH1 and the second CL comprise the following amino acid substitutions: T139D in CH1 and S114K in CL.

In some embodiments, the first CH1 and the first CL comprise the following amino acid substitutions:
(a) C220A in CH1 and C214A in CL;
(b) P171C in CH1 and S165C in CL; and
(c) T139D in CH1 and S114K in CL;
   and the second CH1 and the second CL comprise the following amino acid substitutions: T139R in CH1 and S114E in CL.

In some embodiments, when the first CH1 and the first CL comprise natural-non-cysteine-to-cysteine amino acid substitutions, the second CH1 and the second CL do not comprise natural-non-cysteine-to-cysteine amino acid substitutions and retain the natural cysteines 220C in CH1 and 214C in CL.

In some embodiments, the first CL is from an antibody κ light chain (Cκ); the second CL is from an antibody λ light chain (Cλ) or κ light chain (Cκ). In some embodiments, the first CL is from a κ light chain and the second CL is from a λ light chain.

In some embodiments, the antigen-binding protein further comprises a Fc region comprising a first subunit Fc1 and a second subunit Fc2 capable of associating with each other. In some embodiments, the Fc region is selected from the group consisting of the Fc of human IgG1, IgG2, IgG3 and IgG4, e.g., the Fc of human IgG1.

In some embodiments, Fc1 and Fc2 comprise such amino acid substitutions that Fc1 is preferentially paired with Fc2 over Fc1 (or that a heterodimer is preferentially formed); for example, Fc1 and Fc2 comprise such amino acid substitutions in CH3 domains. In some embodiments, the amino acid substitutions in Fc1 and Fc2 result in greater electrostatic complementarity than a wild type without the substitutions. Methods for measuring electrostatic complementarity at a protein/protein interface are known in the art and are described, for example, in McCoy et al., (1997) J Mol Biol 268,570-584; Lee et al., (2001) Protein Sci. 10,362-377; and Chau et al., (1994) J Comp Mol Des 8,51325. In some embodiments, the amino acid substitutions in Fc1 and Fc2 result in greater steric complementarity than a wild type without the substitutions. Methods for measuring electrostatic complementarity at a protein/protein interface are known in the art and are described, for example, in Lawrence et al., (1993) J Mol Biol 234,946-950; Walls et al., (1992) J Mol Biol 228,277-297; and Schueler-Furman et al., (2005) Proteins 60,187-194. The term “complementarity” refers to, for example, a combination of interactions that affect heavy/light chain pairing at the interface of CH1 and CL (or CH3 and CH3) of the antigen-binding protein described herein. “Steric complementarity” or “conformational complementarity” refers to, for example, the compatibility of three-dimensional structures at the interaction surface of CH1 and CL (or CH3 and CH3). “Electrostatic complementarity” refers to, for example, the compatibility of negatively and/or positively charged atoms placed at the interaction surface of CH1 and CL (or CH3 and CH3).

In some embodiments, in Fc1 and Fc2, for example, within the CH3/CH3 interface, one or more amino acid residues in the CH3 domain of Fc1 are substituted with one or more amino acid residues with larger side-chain volumes, thereby forming protuberances (or knobs) in the surface of the CH3 domain of Fc1; one or more, preferably two or three, of the amino acid residues in the CH3 domain of Fc2 that interact with the CH3 domain of Fc1 are substituted with amino acid residues with small side-chain volumes, thereby forming cavities (or holes) in the surface of the CH3 domain of Fc2 that interacts with the CH3 domain of Fc1. In some embodiments, the CH3 domains of Fc1 and Fc2 (e.g., Fc1 and Fc2 in any of the embodiments described herein) are altered such that within the interface, one or two amino acid residues in the CH3 domain of Fc2 are substituted with an equivalent number of amino acid residues with larger side-chain volumes, thereby forming within the interface of the CH3 domain of Fc2 protuberances (or knobs) that are positionable in cavities (or holes) within the surface of the CH3 domain of Fc1; the CH3 domain of Fc1 is altered such that within the surface of the CH3 domain of Fc2 in contact with the interface of the CH3 domain of Fc2, two or three amino acid residues are substituted with an equivalent number of amino acid residues with smaller side-chain volumes, thereby forming within the interface of the CH3 domain of Fc1 cavities in which protuberances within the interface of the CH3 domain of Fc2 are positionable. In some embodiments, an import residue with a larger side-chain volume is phenylalanine (F), tyrosine (Y), arginine (R) or tryptophan (W). In some embodiments, the protuberance or knob mutations include a substitution of threonine at position 366 with tryptophan; amino acids are numbered according to the EU numbering scheme of Kabat et al. (Sequences of proteins of immunological interest, 5th Edition, Volume 1 (1991; NIH, Bethesda, MD), pp. 688-696). In some embodiments, an import residue with a smaller side-chain volume is serine (S), alanine (a), valine (V) or threonine (T). In one embodiment, a CH3 domain comprising cavities comprises substitutions of two or more original amino acids selected from the group consisting of threonine, leucine and tyrosine. In some embodiments, a CH3 domain comprising cavities comprises two or more import residues selected from the group consisting of alanine, serine, threonine and valine. In some embodiments, a knob mutation modification is T366W, and hole mutation modifications are at least one or at least two of T366S, L368A and Y407V. In some embodiments, a knob mutation modification is T366W, and hole mutation modifications are T366S, L368A and Y407V.

In the context of the present disclosure, the positions of amino acid substitutions in Fc are numbered according to the EU numbering scheme, for example, on the basis of the Fc of human IgG1.

In some embodiments, Fc1 and Fc2 may comprise natural-non-cysteine-to-cysteine substitutions, for example, in CH3; for example, Fc1 comprises S354C, and Fc2 comprises Y349C; or Fc1 comprises Y349C, and Fc2 comprises S354C.

In some embodiments, Fc1 and/or the Fc2 comprise(s) a modification that alters the half-life of the antigen-binding protein, wherein the half-life is dependent on FcRn binding affinity.

In some embodiments, Fc1 and/or the Fc2 comprise(s) a modification that alters effector functions, wherein binding affinity for Fcγreceptors or C1q complement protein is increased or decreased.

In some embodiments, Fc1 and Fc2 comprise, for example, within the Fc1 CH3/Fc2 CH3 interface, amino acid substitutions selected from one or more of the following:
(1) T366Y/Y407T;
(2) T366W/Y407A;
(3) T366Y/Y407T;
(4) T394W/F405A;
(5) T366Y/F405AT394W/Y407T;
(6) T366W/F405WT394S/Y407A;
(7) F405W/T394S;
(8) D399C/K392C;
(9) T366W/T366S/L368A/Y407V;
(10) T366W/D399C/T366S/L368A/K392C/Y407V;
(11) T366W/K392C/T366S/D399C/L368A/Y407V;
(12) S354C/T366W/Y349C/T366S/L368A/Y407V;
(13) Y349C/T366W/S354C/T366S/L368A/Y407V;
(14) E356C/T366W/Y349C/T366S/L368A/Y407V;
(15) Y349C/T366W/E356C/T366S/L368A/Y407V;
(16) E357C/T366W/Y349C/T366S/L368A/Y407V; and
(17) Y349C/T366W/E357C/T366S/L368A/Y407V.

In some embodiments, the Fc1 comprises at least one or at least two amino acid substitutions selected from the group consisting of T366S, L368A and Y407V, and the Fc2 comprises T366W; or the Fc1 comprises T366W, and the Fc2 comprises at least one or at least two amino acid substitutions selected from the group consisting of T366S, L368A and Y407V.

In some embodiments, the Fc1 comprises amino acid substitutions T366S, L368A and Y407V, and the Fc2 comprises T366W; or the Fc1 comprises T366W, and the Fc2 comprises amino acid substitutions T366S, L368A and Y407V.

In some embodiments, Fc1 and Fc2 also comprise amino acid substitutions that cause an electrostatic interaction interface to be formed between Fc1 and Fc2 (e.g., CH3 and CH3). The amino acid substitutions that cause an electrostatic interaction interface to be formed may be selected from one or more of the following:
(1) K370E/D399K/K439D/D356K/E357K/K409D;
(2) K409D/D399K;
(3) K409E/D399K;
(4) K409E/D399R;
(5) K409D/D399R;
(6) D339K/E356K;
(7) D399K/E356K/K409D/K392D;
(8) D399K/E356K/K409D/K439D;
(9) D399K/E357K/K409D/K370D;
(10) D399K/E356K/E357K/K409D/K392D/K370D;
(11) D399K/E357K/K409D/K392D;
(12) K392D/K409D/D399K; and
(13) K409D/K360D/D399K.

In some embodiments, Fc1 and/or Fc2 comprise(s) domains from different antibody subtypes, e.g., CH3 from different antibody subtypes. For example, Davis et al. (2010, Protein Engineering, Design and Selection, 23:195-202) described a Fc platform of a heterodimer that uses a strand-exchange engineered domain (SEED) CH3 region, and the CH3 region is a derivative of human IgG and the IgA CH3 domain (see also WO 2007/110205).

In some embodiments, Fc1 and/or Fc2 comprise(s) amino acid substitutions for altering effector functions, for example, in CH3. “Effector function” refers to those biological activities which are attributable to the Fc region (a natural sequence Fc region or amino acid sequence variant Fc region) of an antibody and which vary with the antibody isotype. Examples of antibody effector functions include: C1q binding and complement-dependent cytotoxicity, Fc receptor binding, antibody-dependent cellular cytotoxicity (ADCC), phagocytosis, down-regulation of cell surface receptors (e.g., B-cell receptor), and B cell activation. The amino acid substitutions that alter effector functions are selected from one or more of the following:
(1) S298A/E333A/K334A;
(2) S239D/I332E/A330L;
(3) S239D/I332E/G236A;
(4) G236A/S239D/A330L/I332E;
(5) F243L/R292P/Y300LN3051/P396L;
(6) K326A/E333A;
(7) K326W/E333S;
(8) K326M/E333S;
(9) C221D/D222C;
(10) S267E/H268F/S324T;
(11) E345R;
(12) S298A/E333A/K334A/N434A;
(13) E294 deletion/T307P/N434Y;
(14) T256N/A378V/S383N/N434Y;
(15) T252L/T253S/T254F;
(16) M252Y/S254T/T256E;
(17) M428L/N434S;
(18) L234A/L235A;
(19) S228P/L235E;
(20) L234A/L235A/P331S;
(21) L234A/L235A/P329G;
(22) D265A/E233P;
(23) H268QN309L/A330S/P331S;
(24) V234A/G237A/P238S/H268A/V309L/A300S/P331S;
(25) L234A/L235A/G237A/P238S/H268A/V309L/A300S/P331S;
(26) S228P/F234A/L235A;
(27) D270A/P329A;
(28) L234F/L235E;
(29) L234F/L235E/P331S;
(30) F241A/V264A/D265A;
(31) N297G/D265A; and
(32) L234Y/G236W/S298A.

In some embodiments, the Fc1 and/or the Fc2 comprise(s) amino acid substitutions L234A and L235A, or comprise(s) amino acid substitutions L234F and L235E.

In some embodiments, Fc1 and/or Fc2 comprise(s) one or more isoallotype mutations, for example, in CH3. In some embodiments, the isoallotype mutations are D356E and L358M.

In some embodiments, Fc1 and Fc2 comprise amino acid substitutions for altering the half-life, for example, in CH3. An increase in half-life can allow for a reduction in the amount of a drug given to a patient and a reduction in the frequency of administration. Accordingly, the antibodies herein with increased half-lives may be generated by modifying (for example, substituting, deleting or adding) amino acid residues identified as being involved in the interaction between the Fc and the FcRn receptor (U.S.7,083,784). In some aspects, a methionine at position 252, and/or a serine at position 254 and/or a threonine at position 256 of an IgG1 isotype antibody can be changed to tyrosine, threonine and glutamic acid, respectively, such that the resulting antibody comprises tyrosine-252, threonine-254 and glutamic acid-256 (i.e., M252Y, S254T and T256E). Such a Fc region of an IgG1 antibody comprises a YTE modification and counterpart positions can be similarly modified in IgG2, IgG3 and IgG4 antibodies. In addition, the half-life of the antibody herein may be increased by conjugation to PEG or albumin using techniques known in the art. In some aspects, the Fc modifications for increasing heterodimer formation may be combined with other modifications for altering the half-life of the antibody, including but not limited to M252Y and/or S254T and/or T256E; and/or with other known Fc modifications for altering effector functions and/or altering binding to one or more Fc ligands, including those described herein.

In some embodiments, the antigen-binding protein provided by the present disclosure comprises a first heavy chain, a first light chain, a second heavy chain and a second light chain, wherein:
the first heavy chain is [VH1]-[first CH1]-[Fc 1] in order from N-terminus to C-terminus;
the first light chain is [VL1]-[first CL] in order from N-terminus to C-terminus; the second heavy chain is [VH2]-[second CH1]-[Fc2] in order from N-terminus to C-terminus;
the second light chain is [VL2]-[second CL] in order from N-terminus to C-terminus.

In some embodiments, the antigen-binding protein provided by the present disclosure comprises a heavy chain, a first light chain and a second light chain, wherein:
the heavy chain is [VH1]-[first CH1]-[Fc1]-[linker]-[VH2]-[second CH1] in order from N-terminus to C-terminus;
the first light chain is [VL1]-[first CL] in order from N-terminus to C-terminus; the second light chain is [VL2]-[second CL] in order from N-terminus to C-terminus.

In some embodiments, the antigen-binding protein provided by the present disclosure comprises a first heavy chain, a first light chain, a second heavy chain and a second light chain, wherein:
the first heavy chain is [VH1]-[first CH1]-[Fc1]-[linker]-[VH2]-[second CH1] in order from N-terminus to C-terminus;
the first light chain is [VL1]-[first CL] in order from N-terminus to C-terminus;
the second heavy chain is [VH1]-[first CH1]-[Fc2]-[linker]-[VH2]-[second CH1] in order from N-terminus to C-terminus;
the second light chain is [VL2]-[second CL] in order from N-terminus to C-terminus.

In some embodiments, antigens to which the first antigen-binding domain and/or the second antigen-binding domain bind(s) include, but are not limited to: PD-1; PD-L1; CTLA-4; LAG-3; OX40; GTIR; A2AR; B7-H3 (CD276); B7-H3; B7-H4; IDO; KIR; Tim-3; LAG-3; 4-IBB (CD137); BAFF; folate receptor 1; TEM1; CCR4; VISTA; ICOS; IFN-γ; TGF-B; EGFR; Erb(ErbB 1; ErbB3; ErbB4); HER2; TNF-α; TNF-(3; TNF-γ; TNF-receptor; BCMA; RANK; VEGF-A; VEGF-B; VEGFR; ROR1; BTLA; 2B4; TIGIT; c-Met; GITR; FAP; PVRIG; BCMA; CAIX; CEA; EGP2; EGP-40; TROP-2; EpCAM; folate-binding protein (1-BP); fetal acetylcholine receptor (AChR); ganglioside G2 (GD2); ganglioside G3 (GD3); human telomerase reverse transcriptase (hTERT); Lewis A (CA 1.9.9); Lewis Y (LeY); GPC3; L1CAM; NG2D ligand; oncofetal antigen (h5T4); prostate stem cell antigen (PSCA); prostate specific membrane antigen (PSMA); TAG-72; CLDN18.2; Wilms tumor protein (WT-1); ROR1; members of the mucin family (e.g., MUC1, MUC2, MUC3A, MUC3B, MUC4, MUC5AC, MUC5B, MUC6, MUC7, MUC8, MUC12, MUC13, MUC15, MUC16, MUC17, MUC19 and MUC20); interleukins and receptors thereof (e.g., IL-1; IL-la; IL-1(3; IL-2; IL-2R; IL-3; IL-4; IL-5; IL-4; IL-4R; IL-6; IL-6R; IL-7; IL-8; IL-9; IL-11; IL-12; IL-12β; IL-13; IL13Rα2; IL-15; IL-15R; IL-17; IL-18; IL-23; and IL-23a); leukocyte differentiation antigens (e.g., CD3; CD4; CDS; CD6; CD7; CD8; CD10; CD14; CD15; CD19; CD20; CD21; CD22; CD23; CD24; CD25; CD26; CD27; CD28; CD30; CD33; CD34; CD36; CD37; CD38; CD40; CD41; CD44; CD45; CD46; CD47; CD51; CD52; CD53; CD54; CD56; CD66; CD70; CD74; CD79a/CD79b; CD80; CD92; CD103; CD122; CD123; CD126; CD133; CD138; CD147; CD148; CD150; CD152; CD171; CD261; CD262; CD317; and CD362); CA125; mesothelin; interferon A/B receptor; HLA-DR; RTN4; VWF; MCP-1; EGFR; IGF-1R; TRAIL-R2; insulin-like growth factor 1 receptor; DLL4; ILGF2; SLAMF7; TWEAKR; CD54; interferon receptors; integrin Avβ3; HNGF; HGF; TYRP1; IGF-1; Cldn18.2; selectin P; SDC1; PDCD1; CFD; hepatitis B surface antigen; IGHE; KIR2D; TAG-72; CSF2; RON; angiopoietin 2; CDK4; CEACAMS/CEACAM6; CO17-1A; CO-43 (blood group Leb); CO-514 (blood group Lea); CTA-1; cytokeratin 8; D1.1; D156-22; DRS; GAGE (GAGE-1 and GAGE-2); GICA 19-9; gp100; Gp37 (human leukemia T cell antigen); gp75 (melanoma antigen); gpA33; HMFG (human milk fat globule antigen); human papillomavirus-E6/human papillomavirus-E7; HMW-MAA (high molecular weight melanoma antigen); I antigen; integrin β6; KIDS; KID31; KS1/4 pan-antigen; L6 and L20 (human lung carcinoma antigens); LEA; LUCA-2; M18; M39; MAGE (MAGE-1 and MAGE-3); MART; Myl; N-acetylglucosaminyltransferase; neoglycoprotein; NS-10; OFA-1; OFA-2; oncostatin M; p15; p97; PEM (polymorphic epithelial mucin); PEMA (polymorphic epithelial mucin antigen); PIPA; PSA (prostate specific antigen); prostatic acid phosphatase (PAP); R24 found in melanoma; stage specific embryonic antigens (e.g., SSEA-1; SSEA-3; and SSEA-4); T5A7; TAG-72; TL5 (blood group A); TRA-1-85 (blood group H); transferrin receptor; C-type lectin-like molecule-1 (CLL-1 or CLECL1); 6-like 3 (DLL3); epidermal growth factor receptor variant III (EGFRvIII); n antigens ((Tn Ag) or (GaINAcu-Ser/Thr)); Fms-like tyrosine kinase 3 (FLT3); protease serine 21 (testisin or PRSS21); PDGFR-(3; neural cell adhesion molecule (NCAM); mutated elongation factor 2 (ELF2M); ephrin B2; proteasome (prosome, macropain) subunit, β type, 9 (LMP2); glycoprotein 100 (gp100); oncogene fusion protein consisting of breakpoint cluster region (BCR) and Abelson murine leukemia viral oncogene homolog 1 (AB1) (bcr-abl); tyrosinase; ephrin type-A receptor 2 (EphA2); fucosyl-GM1; transglutaminase 5 (TGSS); STEAP1; Claudin 6; thyroid stimulating hormone receptor (TSHR); CXORF61; ALK; polysialic acid; PLAC1; mammary gland differentiation antigen (NY-BR-1); uroplakin 2 (UPK2); hepatitis A virus cellular receptor 1 (HAVCR1); adrenoceptor (33 (ADRB3); pannexin 3 (PANX3); G protein-coupled receptor 20 (GPR20); LY6K; OR51E2; cancer/testis antigen 1 (NY-ESO-1); cancer/testis antigen 2 (LAGE-1A); melanoma-associated antigen 1 (MAGE-A1); ETV6-AML; SPA17; XAGE1; Tie2; MAD-CT-1; MAD-CT-2; FOS-related antigen 1; tumor protein p53 (p53); p53 mutant; PCTA-1; hTERT; melanoma inhibitor of apoptosis (ML-IAP); PAX3; androgen receptor; cyclin B1; MYCN; RhoC; TRP-2; CYP1B1 ; SART3; PAX5 ; lymphocyte-specific protein tyrosine kinase (LCK); RAGE-1; RUl; RU2; HPV E6; HPV E7; LAIR1; LILRA2; bone marrow stromal cell antigen 2 (BST2); EGF-like module-containing mucin-like hormone receptor-like 2 (EMR2); lymphocyte antigen 75 (LY75); glypican-3 (GPC3); Fc receptor-like 5 (FCRLS); and immunoglobulin polypeptide 1 (IGLL1).

In some embodiments, the first antigen-binding domain specifically binds to CTLA-4, and the second antigen-binding domain specifically binds to PD-1; or, the first antigen-binding domain specifically binds to PD-1, and the second antigen-binding domain specifically binds to CTLA-4.

In some embodiments, the first antigen-binding domain comprises a heavy chain variable region VH1 and a light chain variable region VL1, and the second antigen-binding domain comprises a heavy chain variable region VH2 and a light chain variable region VL2, wherein the VH1 comprises: a HCDR1 with a sequence set forth in SEQ ID NO: 51, a HCDR2 with a sequence set forth in SEQ ID NO: 52, and a HCDR3 with a sequence set forth in SEQ ID NO: 53, and the VL1 comprises a LCDR1 with a sequence set forth in SEQ ID NO: 54, a LCDR2 with a sequence set forth in SEQ ID NO: and a LCDR3 with a sequence set forth in SEQ ID NO: 56; and/or the VH2 comprises: a HCDR1 with a sequence set forth in SEQ ID NO: 43, a HCDR2 with a sequence set forth in SEQ ID NO: 44, and a HCDR3 with a sequence set forth in SEQ ID NO: 45, and the VL2 comprises a LCDR1 with a sequence set forth in SEQ ID NO: 46, a LCDR2 with a sequence set forth in SEQ ID NO: 47, and a LCDR3 with a sequence set forth in SEQ ID NO: 48.

In some embodiments, the VH1 is a heavy chain variable region with a sequence set forth in SEQ ID NO: 57, and the VL1 is a light chain variable region with a sequence set forth in SEQ ID NO: 58; and/or the VH2 is a heavy chain variable region with a sequence set forth in SEQ ID NO: 49, and the VL2 is a light chain variable region with a sequence set forth in SEQ ID NO: 50.

In one embodiment, the antigen-binding protein of the present disclosure comprises: a first heavy chain with a sequence set forth in SEQ ID NO: 18, a first light chain with a sequence set forth in SEQ ID NO: 17, a second heavy chain with a sequence set forth in SEQ ID NO: 12, and a second light chain with a sequence set forth in SEQ ID NO: 13.

In one embodiment, the antigen-binding protein of the present disclosure comprises: a first heavy chain with a sequence set forth in SEQ ID NO: 19, a first light chain with a sequence set forth in SEQ ID NO: 20, a second heavy chain with a sequence set forth in SEQ ID NO: 12, and a second light chain with a sequence set forth in SEQ ID NO: 13.

In one embodiment, the antigen-binding protein of the present disclosure comprises: a first heavy chain with a sequence set forth in SEQ ID NO: 21, a first light chain with a sequence set forth in SEQ ID NO: 22, a second heavy chain with a sequence set forth in SEQ ID NO: 12, and a second light chain with a sequence set forth in SEQ ID NO: 13.

In one embodiment, the antigen-binding protein of the present disclosure comprises: a first heavy chain with a sequence set forth in SEQ ID NO: 14, a first light chain with a sequence set forth in SEQ ID NO: 15, a second heavy chain with a sequence set forth in SEQ ID NO: 23, and a second light chain with a sequence set forth in SEQ ID NO: 9.

In one embodiment, the antigen-binding protein of the present disclosure comprises: a first heavy chain with a sequence set forth in SEQ ID NO: 14, a first light chain with a sequence set forth in SEQ ID NO: 15, a second heavy chain with a sequence set forth in SEQ ID NO: 24, and a second light chain with a sequence set forth in SEQ ID NO: 9.

In one embodiment, the antigen-binding protein of the present disclosure comprises: a first heavy chain with a sequence set forth in SEQ ID NO: 14, a first light chain with a sequence set forth in SEQ ID NO: 15, a second heavy chain with a sequence set forth in SEQ ID NO: 25, and a second light chain with a sequence set forth in SEQ ID NO: 10.

In one embodiment, the antigen-binding protein of the present disclosure comprises: a first heavy chain with a sequence set forth in SEQ ID NO: 14, a first light chain with a sequence set forth in SEQ ID NO: 15, a second heavy chain with a sequence set forth in SEQ ID NO: 26, and a second light chain with a sequence set forth in SEQ ID NO: 8.

In one embodiment, the antigen-binding protein of the present disclosure comprises: a first heavy chain with a sequence set forth in SEQ ID NO: 14, a first light chain with a sequence set forth in SEQ ID NO: 27, a second heavy chain with a sequence set forth in SEQ ID NO: 12, and a second light chain with a sequence set forth in SEQ ID NO: 13.

In one embodiment, the antigen-binding protein of the present disclosure comprises: a first heavy chain with a sequence set forth in SEQ ID NO: 28, a first light chain with a sequence set forth in SEQ ID NO: 29, a second heavy chain with a sequence set forth in SEQ ID NO: 12, and a second light chain with a sequence set forth in SEQ ID NO: 13.

In one embodiment, the antigen-binding protein of the present disclosure comprises: a first heavy chain with a sequence set forth in SEQ ID NO: 14, a first light chain with a sequence set forth in SEQ ID NO: 27, a second heavy chain with a sequence set forth in SEQ ID NO: 25, and a second light chain with a sequence set forth in SEQ ID NO: 10.

In one embodiment, the antigen-binding protein of the present disclosure comprises: a first heavy chain with a sequence set forth in SEQ ID NO: 14, a first light chain with a sequence set forth in SEQ ID NO: 15, a second heavy chain with a sequence set forth in SEQ ID NO: 31, and a second light chain with a sequence set forth in SEQ ID NO: 32.

In one embodiment, the antigen-binding protein of the present disclosure comprises: a first heavy chain with a sequence set forth in SEQ ID NO: 19, a first light chain with a sequence set forth in SEQ ID NO: 20, a second heavy chain with a sequence set forth in SEQ ID NO: 12, and a second light chain with a sequence set forth in SEQ ID NO: 30.

In one embodiment, the antigen-binding protein of the present disclosure comprises: a first heavy chain with a sequence set forth in SEQ ID NO: 35, a first light chain with a sequence set forth in SEQ ID NO: 36, a second heavy chain with a sequence set forth in SEQ ID NO: 33, and a second light chain with a sequence set forth in SEQ ID NO: 34.

In one embodiment, the antigen-binding protein of the present disclosure comprises: a first heavy chain with a sequence set forth in SEQ ID NO: 14, a first light chain with a sequence set forth in SEQ ID NO: 15, a second heavy chain with a sequence set forth in SEQ ID NO: 25, and a second light chain with a sequence set forth in SEQ ID NO: 10.

In one embodiment, the antigen-binding protein of the present disclosure comprises: a first heavy chain with a sequence set forth in SEQ ID NO: 45, a first light chain with a sequence set forth in SEQ ID NO: 46, a second heavy chain with a sequence set forth in SEQ ID NO: 37, and a second light chain with a sequence set forth in SEQ ID NO: 38.

In one embodiment, the antigen-binding protein of the present disclosure comprises: a first heavy chain with a sequence set forth in SEQ ID NO: 41, a first light chain with a sequence set forth in SEQ ID NO: 42, a second heavy chain with a sequence set forth in SEQ ID NO: 39, and a second light chain with a sequence set forth in SEQ ID NO: 40.

In some embodiments, the first antigen-binding domain specifically binds to CD40, and/or the second antigen-binding domain specifically binds to FAP.

In some embodiments, the first antigen-binding domain comprises a heavy chain variable region VH1 and a light chain variable region VL1, and the second antigen-binding domain comprises a heavy chain variable region VH2 and a light chain variable region VL2, wherein the VH1 comprises: a HCDR1 with a sequence set forth in SEQ ID NO: 73, a HCDR2 with a sequence set forth in SEQ ID NO: 74, and a HCDR3 with a sequence of RDY, and the VL1 comprises a LCDR1 with a sequence set forth in SEQ ID NO: 75, a LCDR2 with a sequence set forth in SEQ ID NO: 76, and a LCDR3 with a sequence set forth in SEQ ID NO: 77; and/or the VH2 comprises: a HCDR1 with a sequence set forth in SEQ ID NO: 80, a HCDR2 with a sequence set forth in SEQ ID NO: 81, and a HCDR3 with a sequence set forth in SEQ ID NO: 82, and the VL2 comprises a LCDR1 with a sequence set forth in SEQ ID NO: 83, a LCDR2 with a sequence set forth in SEQ ID NO: 84, and a LCDR3 with a sequence set forth in SEQ ID NO: 85.

In some embodiments, the VH1 is a heavy chain variable region with a sequence set forth in SEQ ID NO: 78, and the VL1 is a light chain variable region with a sequence set forth in SEQ ID NO: 79; and/or the VH2 is a heavy chain variable region with a sequence set forth in SEQ ID NO: 86, and the VL2 is a light chain variable region with a sequence set forth in SEQ ID NO: 87.

In one embodiment, the antigen-binding protein of the present disclosure comprises: a first heavy chain with a sequence set forth in SEQ ID NO: 67, a first light chain with a sequence set forth in SEQ ID NO: 68, and a second light chain with a sequence set forth in SEQ ID NO: 69.

In one embodiment, the antigen-binding protein of the present disclosure comprises: a first heavy chain with a sequence set forth in SEQ ID NO: 70, a first light chain with a sequence set forth in SEQ ID NO: 71, and a second light chain with a sequence set forth in SEQ ID NO: 72.

In some embodiments, the first antigen-binding domain specifically binds to a different epitope of PSMA from the second antigen-binding domain.

In some embodiments, the antigen-binding protein of the present disclosure comprises: a first heavy chain with a sequence set forth in SEQ ID NO: 59, a first light chain with a sequence set forth in SEQ ID NO: 60, a second heavy chain with a sequence set forth in SEQ ID NO: 61, and a second light chain with a sequence set forth in SEQ ID NO: 62.

In some embodiments, the antigen-binding protein of the present disclosure comprises: a first heavy chain with a sequence set forth in SEQ ID NO: 63, a first light chain with a sequence set forth in SEQ ID NO: 64, a second heavy chain with a sequence set forth in SEQ ID NO: 65, and a second light chain with a sequence set forth in SEQ ID NO: 66.

The present disclosure provides a PD-1/CTLA-4 bispecific antibody comprising:
(i) a PD-1 antigen-binding domain comprising a first light chain and a first heavy chain, wherein the CH1 of the first heavy chain and the CL of the first light chain comprise the following amino acid substitutions:
   (a) C220A in CH1 and C214A in CL;
   (b) amino acid substitutions selected from at least one of the following:
      (b-1) F170C in CH1 and T164C in CL;
      (b-2) L128C in CH1 and S121C in CL;
      (b-3) A129C in CH1 and S121C in CL;
      (b-4) S131C in CH1 and P119C in CL;
      (b-5) A141C in CH1 and L135C in CL; and
      (b-6) P171C in CH1 and S165C in CL; and
   (c) amino acid substitutions selected from any one of the following:
      (c-1) T139R in CH1 and S114E in CL;
      (c-2) T139R in CH1 and S114D in CL;
      (c-3) T139K in CH1 and S114E in CL; and
      (c-4) T139K in CH1 and S114D in CL; and
(ii) a CTLA-4 antigen-binding domain comprising a second light chain and a second heavy chain, wherein the CH1 of the second heavy chain and the CL of the second light chain comprise amino acid substitutions selected from any one of the following:
   (1) T139D in CH1 and S114K in CL;
   (2) T139D in CH1 and S114R in CL;
   (3) T139E in CH1 and S114K in CL; and
   (4) T139E in CH1 and S114R in CL.

The present disclosure provides a PD-1/CTLA-4 bispecific antibody comprising:
(i) a PD-1 antigen-binding domain comprising a first light chain and a first heavy chain, wherein the CH1 of the first heavy chain and the CL of the first light chain comprise amino acid substitutions selected from any one of the following:
   (1) T139D in CH1 and S114K in CL;
   (2) T139D in CH1 and S114R in CL;
   (3) T139E in CH1 and S114K in CL; and
   (4) T139E in CH1 and S114R in CL; and
(ii) a CTLA-4 antigen-binding domain comprising a second light chain and a second heavy chain, wherein the CH1 of the second heavy chain and the CL of the second light chain comprise the following amino acid substitutions:
   (a) C220A in CH1 and C214A in CL;
   (b) amino acid substitutions selected from at least one of the following:
      (b-1) S131C in CH1 and P119C in CL;
      (b-2) L128C in CH1 and S121C in CL;
      (b-3) A129C in CH1 and S121C in CL;
      (b-4) F170C in CH1 and T164C in CL;
      (b-5) A141C in CH1 and L135C in CL; and
      (b-6) P171C in CH1 and S165C in CL; and
   (c) amino acid substitutions selected from any one of the following:
      (1) T139R in CH1 and S114E in CL;
      (2) T139R in CH1 and S114D in CL;
      (3) T139K in CH1 and S114E in CL; and
      (4) T139K in CH1 and S114D in CL.

The present disclosure provides a FAP/CD40 bispecific antibody comprising:
(i) a CD40 antigen-binding domain comprising a first light chain and a first heavy chain, wherein the CH1 of the first heavy chain and the CL of the first light chain comprise the following amino acid substitutions:
   (a) C220A in CH1 and C214A in CL;
   (b) amino acid substitutions selected from at least one of the following:
      (b-1) F170C in CH1 and T164C in CL;
      (b-2) L128C in CH1 and S121C in CL;
      (b-3) A129C in CH1 and S121C in CL;
      (b-4) S131C in CH1 and P119C in CL;
      (b-5) A141C in CH1 and L135C in CL; and
      (b-6) P171C in CH1 and S165C in CL; and
(ii) a FAP antigen-binding domain comprising a second light chain and a second heavy chain.

The present disclosure provides a FAP/CD40 bispecific antibody comprising:
(i) a CD40 antigen-binding domain comprising a first light chain and a first heavy chain; and
(ii) a FAP antigen-binding domain comprising a second light chain and a second heavy chain, wherein the CH1 of the second heavy chain and the CL of the second light chain comprise the following amino acid substitutions:
   (a) C220A in CH1 and C214A in CL;
   (b) amino acid substitutions selected from at least one of the following:
      (b-1) F170C in CH1 and T164C in CL;
      (b-2) L128C in CH1 and S121C in CL;
      (b-3) A129C in CH1 and S121C in CL;
      (b-4) S131C in CH1 and P119C in CL;
      (b-5) A141C in CH1 and L135C in CL; and
      (b-6) P171C in CH1 and S165C in CL.

In some embodiments, the first heavy chain and the second heavy chain are linked by a linker. In some embodiments, the peptide linker is a peptide with an amino acid sequence of at least 5 amino acids; in one embodiment, the peptide linker is a peptide with an amino acid sequence of 5 to 100 amino acids; in a further embodiment, the peptide linker is a peptide with an amino acid sequence of 10 to 50 amino acids. In one embodiment, the peptide linker is (GxS)n or (GxS)nGm, wherein G=glycine, S=serine, and (x=3, n=3, 4, 5 or 6, and m=0, 1, 2 or 3) or (x=4, n=2, 3, 4 or 5, and m=0, 1, 2 or 3). In one embodiment, x=4 and n=3 or 4. In one embodiment, the peptide linker is (G4S)4.

The present disclosure provides a PSMA-binding biepitopic antibody comprising:
(i) a first light chain and a first heavy chain that bind to a first epitope, wherein the CH1 of a second heavy chain and the CL of a second light chain comprise the following amino acid substitutions:
   (a) C220A in CH1 and C214A in CL;
   (b) amino acid substitutions selected from at least one of the following:
      (b-1) S131C in CH1 and P119C in CL;
      (b-2) L128C in CH1 and S121C in CL;
      (b-3) A129C in CH1 and S121C in CL;
      (b-4) F170C in CH1 and T164C in CL;
      (b-5) A141C in CH1 and L135C in CL; and
      (b-6) P171C in CH1 and S165C in CL; and
(ii) a second light chain and a second heavy chain that bind to a second epitope.

The present disclosure provides a PSMA-binding biepitopic antibody comprising:
(i) a first light chain and a first heavy chain that bind to a first epitope; and
(ii) a second light chain and a second heavy chain that bind to a second epitope, wherein the CH1 of the second heavy chain and the CL of the second light chain comprise the following amino acid substitutions:
   (a) C220A in CH1 and C214A in CL;
   (b) amino acid substitutions selected from at least one of the following:
      (b-1) S131C in CH1 and P119C in CL;
      (b-2) L128C in CH1 and S121C in CL;
      (b-3) A129C in CH1 and S121C in CL;
      (b-4) F170C in CH1 and T164C in CL;
      (b-5) A141C in CH1 and L135C in CL; and
      (b-6) P171C in CH1 and S165C in CL.

The present disclosure provides an antigen-binding protein comprising:
(i) a first antigen-binding domain comprising a polypeptide H1 and a polypeptide Ll, wherein the polypeptide H1 comprises a first CH1 linked to a first VH, and the polypeptide L1 comprises a first CL linked to a first VL, wherein the first CH1 and the first CL comprise natural-non-cysteine-to-cysteine amino acid substitutions at positions selected from one or more of (i-1) to (i-6):
   (i-1) position 170 of the first CH1 and position 164 of the first CL,
   (i-2) position 128 of the first CH1 and position 121 of the first CL,
   (i-3) position 129 of the first CH1 and position 121 of the first CL,
   (i-4) position 131 of the first CH1 and position 119 of the first CL,
   (i-5) position 141 of the first CH1 and position 135 of the first CL, and
   (i-6) position 171 of the first CH1 and position 165 of the first CL; and
(ii) a second antigen-binding domain comprising a polypeptide H2 and a polypeptide L2, wherein the polypeptide H2 comprises a second CH1 linked to a second VH, and the polypeptide L2 comprises a second CL linked to a second VL.
   In some embodiments, the polypeptide H1 comprises a first CH1 linked to a first heavy chain variable region VH1; the polypeptide L1 comprises a first CL linked to a first light chain variable region VL1.

In some embodiments, the polypeptide H1 comprises VH1 and the first CH1 in order from N-terminus to C-terminus; the polypeptide L1 comprises VL1 and the first CL in order from N-terminus to C-terminus.

In some embodiments, the polypeptide H1 comprises VH1, the first CH1 and Fc 1 in order from N-terminus to C-terminus; the polypeptide L1 comprises VL1 and the first CL in order from N-terminus to C-terminus.

In some embodiments, the polypeptide H1 is a first heavy chain, and the polypeptide L1 is a first light chain.

In some embodiments, the polypeptide H2 comprises a second CH1 linked to a second heavy chain variable region VH2; the polypeptide L2 comprises a second CL linked to a second light chain variable region VL2.

In some embodiments, the polypeptide H2 comprises VH2 and the second CH1 in order from N-terminus to C-terminus; the polypeptide L2 comprises VL2 and the second CL in order from N-terminus to C-terminus.

In some embodiments, the polypeptide H2 comprises VH2, the second CH1 and Fc2 in order from N-terminus to C-terminus; the polypeptide L2 comprises VL2 and the second CL in order from N-terminus to C-terminus.

In some embodiments, the polypeptide H2 is a second heavy chain, and the polypeptide L2 is a second light chain.

In some embodiments, the polypeptide H1 and the polypeptide H2 may be linked by a linker. In some embodiments, the polypeptide H1 and the polypeptide H2 that are linked by a linker are [VH1]-[first CH1]-Fc1-[linker]-[VH2]-[secondCH1] in order from N-terminus to C-terminus.

In some embodiments, the first CH1, the first CL, the second CH1 and the second CL are as defined above.

In some embodiments, the polypeptide L1 is an antibody light chain, e.g., a human IgG antibody light chain, which is a κ light chain (Cκ); the polypeptide L2 is an antibody light chain, e.g., a human IgG antibody light chain, which may be a λ light chain (Cλ) or κ light chain (Cκ). In some embodiments, the polypeptide L1 is a κ light chain and the polypeptide L2 is a λ light chain.

In some embodiments, the polypeptide H1 comprises Fc1, the polypeptide H2 comprises Fc2, and the Fc1 and/or the Fc2 are/is selected from the group consisting of the Fc of human IgG1, IgG2, IgG3 and IgG4, for example, the Fc of human IgG1.

In some embodiments, Fc1 and Fc2 are engineered, or modified or substituted with amino acids, as defined above.

In some embodiments, Fc1 and/or the Fc2 comprise(s) a modification that alters the half-life of the antigen-binding protein, wherein the half-life is dependent on FcRn binding affinity.

In some embodiments, Fc1 and/or the Fc2 comprise(s) a modification that alters effector functions, wherein binding affinity for Fcγreceptors or Clq complement protein is increased or decreased.

In some embodiments, Fc 1 and Fc2 comprise such amino acid substitutions that Fc1 is preferentially paired with Fc2 over Fc1.

In some embodiments, the polypeptide L1 comprises amino acid replacements: S165C and C214A, and the polypeptide H1 comprises amino acid replacements: P171C, C220A, L234A, L235A, D356E, L358M, Y349C, T366S, L368A and Y407N; and the polypeptide H2 comprises amino acid replacements: L234A, L235A, D356E, L358M, S354C and T366W; or,
the polypeptide L1 comprises amino acid replacements: S165C and C214A, and the polypeptide H1 comprises amino acid replacements: P171C, C220A, L234A, L235A, D356E, L358M, S354C and T366W; and the polypeptide H2 comprises amino acid replacements: L234A, L235A, D356E, L358M, Y349C, T366S, L368A and Y407N.

In some embodiments, the polypeptide L1 comprises amino acid replacements: T164C, C214A and S114E, and the polypeptide H1 comprises amino acid replacements: T139R, F170C, C220A, L234A, L235A, D356E, L358M, Y349C, T366S, L368A and Y407N; and the polypeptide L2 comprises an amino acid replacement S114K, and the polypeptide H2 comprises amino acid replacements: T139D, L234A, L235A, D356E, L358M, S354C and T366W;
or,
the polypeptide L1 comprises amino acid replacements: T164C, C214A and S114E, and the polypeptide H1 comprises amino acid replacements: T139R, F170C, C220A, L234A, L235A, D356E, L358M, S354C and T366W; and the polypeptide L2 comprises an amino acid replacement S114K, and the polypeptide H2 comprises amino acid replacements: T139D, L234A, L235A, D356E, L358M, Y349C, T366S, L368A and Y407N.

The present disclosure provides a bispecific bivalent antigen-binding protein comprising:
(i) a first antigen-binding domain comprising a polypeptide H1 and a polypeptide Ll, wherein the polypeptide H1 comprises a first CH1 linked to a first VH, and the polypeptide L1 comprises a first CL linked to a first VL, wherein the first CH1 and the first CL each comprise a natural-cysteine-to-non-cysteine amino acid substitution, and the first CH1 and the first CL further comprise natural-non-cysteine-to-cysteine amino acid substitutions at positions selected from the group consisting of:
   (i-1) position 170 of the first CH1 and position 164 of the first CL,
   (i-2) position 128 of the first CH1 and position 121 of the first CL,
   (i-3) position 129 of the first CH1 and position 121 of the first CL,
   (i-4) position 131 of the first CH1 and position 119 of the first CL,
   (i-5) position 141 of the first CH1 and position 135 of the first CL, and
   (i-6) position 171 of the first CH1 and position 165 of the first CL; and
(ii) a second antigen-binding domain comprising a polypeptide H2 and a polypeptide L2, wherein the polypeptide H2 comprises a second CH1 linked to a second VH, and the polypeptide L2 comprises a second CL linked to a second VL; wherein the polypeptide H1 comprises VH, CH1 and Fc in order from N-terminus to C-terminus; the polypeptide H2 comprises VH, CH1 and Fc in order from N-terminus to C-terminus.

The present disclosure provides a bispecific tetravalent antigen-binding protein comprising:
(i) a first antigen-binding domain comprising a polypeptide H1 and a polypeptide L1, wherein the polypeptide H1 comprises a first CH1 linked to a first VH, and the polypeptide L1 comprises a first CL linked to a first VL, wherein the first CH1 and the first CL each comprise a natural-cysteine-to-non-cysteine amino acid substitution, and the first CH1 and the first CL further comprise natural-non-cysteine-to-cysteine amino acid substitutions at positions selected from the group consisting of:
   (i-1) position 170 of the first CH1 and position 164 of the first CL,
   (i-2) position 128 of the first CH1 and position 121 of the first CL,
   (i-3) position 129 of the first CH1 and position 121 of the first CL,
   (i-4) position 131 of the first CH1 and position 119 of the first CL,
   (i-5) position 141 of the first CH1 and position 135 of the first CL, and
   (i-6) position 171 of the first CH1 and position 165 of the first CL; and
(ii) a second antigen-binding domain comprising a polypeptide H2 and a polypeptide L2, wherein the polypeptide H2 comprises a second CH1 linked to a second VH, and the polypeptide L2 comprises a second CL linked to a second VL; wherein: the polypeptide H1 consists of VH and CH1 from N-terminus to C-terminus, the polypeptide H2 comprises VH, CH1 and Fc in order from N-terminus to C-terminus, and the C-terminus of the polypeptide H1 is fused with the C-terminus of the polypeptide H2 optionally through a peptide linker; or the polypeptide H1 comprises VH, CH1 and Fc in order from N-terminus to C-terminus, the polypeptide H2 consists of VH and CH1 from N-terminus to C-terminus, and the C-terminus of the polypeptide H2 is fused with the C-terminus of the polypeptide H1, optionally through a peptide linker.

The peptide linker represents a peptide having an amino acid sequence. In some embodiments, the peptide linker is a peptide with an amino acid sequence of at least 5 amino acids; in one embodiment, the peptide linker is a peptide with an amino acid sequence of 5 to 100 amino acids; in a further embodiment, the peptide linker is a peptide with an amino acid sequence of 10 to 50 amino acids. In one embodiment, the peptide linker is (GxS)n or (GxS)nGm, wherein G=glycine, S=serine, and (x=3, n=3, 4, 5 or 6, and m=0, 1, 2 or 3) or (x=4, n=2, 3, 4 or 5, and m=0, 1, 2 or 3). In one embodiment, x=4 and n=3 or 4. In one embodiment, the peptide linker is (G4S)4.

In some embodiments, the polypeptide H1 consists of VH and CH1 from N-terminus to C-terminus, the polypeptide H2 comprises VH, CH1 and Fc in order from N-terminus to C-terminus, and the C-terminus of the polypeptide H1 is fused with the C-terminus of the polypeptide H2, optionally through a peptide linker.

In some embodiments, the polypeptide H1 comprises VH, CH1 and Fc in order from N-terminus to C-terminus, the polypeptide H2 consists of VH and CH1 from N-terminus to C-terminus, and the C-terminus of the polypeptide H2 is fused with the C-terminus of the polypeptide H1, optionally through a peptide linker.

The present disclosure provides a dimerized polypeptide comprising a heavy chain constant region 1 (CH1) and a light chain constant region (CL), wherein amino acid substitutions that cause an electrostatic interaction interface to be formed between CH1 and CL are comprised at position 139 of CH1 and position 114 of CL.

In some embodiments, the amino acid at position 139 of CH1 is substituted with a positively charged amino acid, and the amino acid at position 114 of CL is substituted with a negatively charged amino acid; or the amino acid at position 139 of CH1 is substituted with a negatively charged amino acid, and the amino acid at position 114 of CL is substituted with a positively charged amino acid.

In some embodiments, the positively charged amino acid is selected from the group consisting of K, R and H; the negatively charged amino acid is selected from the group consisting of D and E.

In some embodiments, CH1 and CL comprise amino acid substitutions selected from the group consisting of: T139R and S114E; T139R and S114D; T139K and S114E; T139K and S114D; T139D and S114K; T139D and S114R; T139E and S114K; and T139E and S114R.

The present disclosure provides an antigen-binding protein comprising the dimerized polypeptide described above.

In some embodiments, the antigen-binding protein comprises a first antigen-binding domain, and the first antigen-binding domain comprises a Fab comprising a first heavy chain variable region VH1, a first light chain variable region VL1, and the dimerized polypeptide; in the dimerized polypeptide, the CH1 is a first CH1, and the CL is a first CL; VH1 and the first CH1 are linked directly or by a linker, and VL1 and the first CL are linked directly or by a linker. In some embodiments, the C-terminus of VH1 and the N-terminus of the first CH1 are linked directly or by a linker, and the C-terminus of VL1 and the N-terminus of the first CL are linked directly or by a linker.

In some embodiments, the antigen-binding protein comprises a first antigen-binding domain and a second antigen-binding domain, wherein the second antigen-binding domain comprises a second heavy chain variable region VH2 and a second light chain variable region VL2, and the first antigen-binding domain and the second antigen-binding domain bind to different antigens or bind to different epitopes on the same antigen; in some embodiments, the second antigen-binding domain comprises a Fab. In some embodiments, the C-terminus of VH2 and the N-terminus of the second CH1 are linked directly or by a linker, and the C-terminus of VL2 and the N-terminus of the second CL are linked directly or by a linker.

In some embodiments, the first CH1 and the first CL comprise amino acid substitutions that cause an electrostatic interaction interface to be formed between the first CH1 and the first CL; and/or

the second CH1 and the second CL comprise amino acid substitutions that cause an electrostatic interaction interface to be formed between the second CH1 and the second CL.

In some embodiments, the amino acids for forming an electrostatic interaction interface in the first CH1 and the second CH1 are oppositely charged, and the amino acids for forming an electrostatic interaction interface in the first CL and the second CL are oppositely charged.

In some embodiments, the amino acid substitutions that cause an electrostatic interaction interface to be formed between the first CH1 and the first CL are at position 139 of the first CH1 and position 114 of the first CL; and/or the amino acid substitutions that cause an electrostatic interaction interface to be formed between the second CH1 and the second CL are at position 139 of the second CH1 and position 114 of the second CL.

In some embodiments, position 139 of the first CH1 and position 139 of the second CH1 are each substituted with an oppositely charged amino acid, and position 114 of the first CL and position 114 of the second CL are each substituted with an oppositely charged amino acid.

In some embodiments, the amino acid at position 139 of the first CH1 is substituted with a positively charged amino acid, and the amino acid at position 114 of the first CL is substituted with a negatively charged amino acid; or the amino acid at position 139 of the first CH1 is substituted with a negatively charged amino acid, and the amino acid at position 114 of the first CL is substituted with a positively charged amino acid; and/or

the amino acid at position 139 of the second CH1 is substituted with a negatively charged amino acid, and the amino acid at position 114 of the second CL is substituted with a positively charged amino acid; or the amino acid at position 139 of the second CH1 is substituted with a positively charged amino acid, and the amino acid at position 114 of the second CL is substituted with a negatively charged amino acid.
In some embodiments, the positively charged amino acid is selected from the group consisting of K, R and H; the negatively charged amino acid is selected from the group consisting of D and E.

In some embodiments, the first CH1 and the first CL comprise amino acid substitutions selected from the group consisting of: T139R and S114E; T139R and S114D; T139K and S114E; T139K and S114D; T139D and S114K; T139D and S114R; T139E and S114K; and T139E and S114R; and/or

the second CH1 and the second CL comprise amino acid substitutions selected from the group consisting of: T139R and S114E; T139R and S114D; T139K and S114E; T139K and S114D; T139D and S114K; T139D and S114R; T139E and S114K; and T139E and S114R.

In some embodiments, the first CH1 and the first CL comprise amino acid substitutions selected from the group consisting of: T139R and S114E; T139R and S114D; T139K and S114E; T139K and S114D; and/or

the second CH1 and the second CL comprise amino acid substitutions selected from the group consisting of: T139D and S114K; T139D and S114R; T139E and S114K; and T139E and S114R.

In some embodiments, the first CH1 and the first CL comprise amino acid substitutions selected from the group consisting of: T139D and S114K; T139D and S114R; T139E and S114K; and T139E and S114R; and/or the second CH1 and the second CL comprise amino acid substitutions selected from the group consisting of: T139R and S114E; T139R and S114D; T139K and S114E; and T139K and S114D.

The present disclosure provides an antigen-binding protein comprising:
(i) a first antigen-binding domain comprising a polypeptide H1 and a polypeptide L1, wherein the polypeptide H1 comprises a first CH1 linked to a first VH, and the polypeptide L1 comprises a first CL linked to a first VL; and
(ii) a second antigen-binding domain comprising a polypeptide H2 and a polypeptide L2, wherein the polypeptide H2 comprises a second CH1 linked to a second VH, and the polypeptide L2 comprises a second CL linked to a second VL;
   wherein: amino acid substitutions that cause an electrostatic interaction interface to be formed between the first CH1 and the first CL are comprised at position 139 of the first CH1 and position 114 of the first CL; and/or
   amino acid substitutions that cause an electrostatic interaction interface to be formed between the second CH1 and the second CL are comprised at position 139 of the second CH1 and position 114 of the second CL.

In some embodiments, the antigen-binding protein is a bispecific bivalent antigen-binding protein, wherein the polypeptide H1 comprises VH, CH1 and Fc in order from N-terminus to C-terminus; the polypeptide H2 comprises VH, CH1 and Fc in order from N-terminus to C-terminus.

In some embodiments, the antigen-binding protein is a bispecific tetravalent antigen-binding protein, wherein the polypeptide H1 consists of VH and CH1 from N-terminus to C-terminus, the polypeptide H2 comprises VH, CH1 and Fc in order from N-terminus to C-terminus, and the C-terminus of the polypeptide H1 is fused with the C-terminus of the polypeptide H2 optionally through a peptide linker; or the polypeptide H1 comprises VH, CH1 and Fc in order from N-terminus to C-terminus, the polypeptide H2 consists of VH and CH1 from N-terminus to C-terminus, and the C-terminus of the polypeptide H2 is fused with the C-terminus of the polypeptide H1 optionally through a peptide linker.

In some embodiments, the antigen-binding protein of the present disclosure is a multispecific antibody, e.g., a bispecific antibody. In some embodiments, the antigen-binding protein of the present disclosure is a chimeric antibody, a humanized antibody or a fully human antibody, a multivalent antibody, or an antibody drug conjugate.

In some embodiments, the antigen-binding protein of the present disclosure comprising the above amino acid substitutions is produced in a single cell with improved polypeptide H1/L1 and polypeptide H2/L2 (e.g., heavy chain/light chain) pairing or in an improved yield, compared to an antigen-binding protein without these amino acid substitutions.

In some embodiments, the proportion of correct pairing of the polypeptide H1/L1 and polypeptide H2/L2 (e.g., heavy chain/light chain) in the antigen-binding protein of the present disclosure is at least 55%, at least 60%, at least 65%, at least 70%, at least 75%, at least 80%, at least 85%, at least 90%, at least 95%, at least 96%, at least 97%, at least 98%, at least 99% or 100%. Calculation formula: proportion of correct pairing of polypeptide H1/L1 and polypeptide H2/L2 (e.g., heavy chain/light chain)=(peak intensity of correct first antigen-binding molecules+peak intensity of correct second antigen-binding molecules)/(peak intensity of correct first antigen-binding molecules+peak intensity of correct second antigen-binding molecules+peak intensity of other impurities)×100%.

In some embodiments, the proportion of correct pairing of the polypeptide H1/L1 and polypeptide H2/L2 (e.g., heavy chain/light chain) in the antigen-binding protein of the present disclosure is increased, relative to a wild type, by at least 5%, at least 10%, at least 15%, at least 20%, at least 25%, at least 30%, at least 35%, at least 40%, at least 45%, at least 46%, at least 47%, at least 48%, at least 49% or 50%.

In some embodiments, by removing a natural disulfide bond from the CH1/CL interface and introducing a non-natural disulfide bond into the interface, the proportion of correct pairing of the polypeptide H1/L1 and polypeptide H2/L2 (e.g., heavy chain/light chain) in the antigen-binding protein of the present disclosure is increased, relative to a wild type, by at least 5%, at least 10%, at least 15%, at least 20%, at least 25%, at least 30%, at least 35%, at least 40%, at least 45%, at least 46%, at least 47%, at least 48%, at least 49% or 50%.

In some embodiments, by introducing a pair of electrostatically complementary amino acids into the CH1/CL interface, the proportion of correct pairing of the polypeptide H1/L1 and polypeptide H2/L2 (e.g., heavy chain/light chain) in the antigen-binding protein of the present disclosure is increased, relative to a wild type, by at least 5%, at least 10%, at least 15%, at least 20%, at least 25%, at least 30%, at least 35%, at least 40%, at least 45%, at least 46%, at least 47%, at least 48%, at least 49% or 50%.

In some embodiments, by removing a natural disulfide bond from the CH1/CL interface and introducing a non-natural disulfide bond and also a pair of electrostatically complementary amino acids into the interface, the proportion of correct pairing of the polypeptide H1/L1 and polypeptide H2/L2 (e.g., heavy chain/light chain) in the antigen-binding protein of the present disclosure is increased, relative to a wild type, by at least 5%, at least 10%, at least 15%, at least 20%, at least 25%, at least 30%, at least 35%, at least 40%, at least 45%, at least 46%, at least 47%, at least 48%, at least 49% or 50%.

The present disclosure further provides a nucleic acid molecule or a combination thereof encoding the aforementioned dimeric polypeptide or antigen-binding protein.

The present disclosure further provides a nucleic acid expression vector or a combination thereof comprising the aforementioned nucleic acid molecule or combination thereof.

The present disclosure further provides a host cell comprising the aforementioned nucleic acid molecule or combination thereof.

In some embodiments, the host cell is any kind of cellular system that can be engineered to produce the dimeric polypeptide or antigen-binding protein according to the present disclosure, such as a eukaryotic or prokaryotic cell. The eukaryotic cell includes, but is not limited to, for example, nucleated cells derived from yeasts, fungi, insects, plants, animals, humans or other multicellular organisms.

The present disclosure further provides a method for preparing any one of the aforementioned dimeric polypeptides or antigen-binding proteins, the method comprising the following steps:
(1) transforming a host cell with the aforementioned nucleic acid expression vector;
(2) culturing the host cell under conditions that permit synthesis of the antigen-binding protein to obtain a cell culture; and
(3) recovering the antigen-binding protein from the cell culture.

In some embodiments, the aforementioned nucleic acid expression vector comprises: a heavy-chain-encoding plasmid and a light-chain-encoding plasmid; in transforming the host cell, the light-chain-encoding plasmid is in excess relative to the heavy-chain-encoding plasmid; for example, the heavy-chain-encoding plasmid and the light-chain-encoding plasmid are in a molar ratio of 1:(1-10), e.g., 1:(1-5), e.g., 2:3.

In some embodiments, the aforementioned nucleic acid expression vector comprises:
a first plasmid comprising a nucleic acid molecule encoding the polypeptide Hl;
a second plasmid comprising a nucleic acid molecule encoding the polypeptide Ll;
a third plasmid comprising a nucleic acid molecule encoding the polypeptide H2; and
a fourth plasmid comprising a nucleic acid molecule encoding the polypeptide L2.

In some embodiments, the aforementioned nucleic acid expression vector comprises:
a first plasmid comprising a nucleic acid molecule encoding the first heavy chain;
a second plasmid comprising a nucleic acid molecule encoding the first light chain;
a third plasmid comprising a nucleic acid molecule encoding the second heavy chain; and
a fourth plasmid comprising a nucleic acid molecule encoding the second light chain. In some embodiments, in transforming the host cell, the first plasmid and the second plasmid are in a molar ratio of 1:1 to 1:10, 1:1 to 1:9, 1:1 to 1:8, 1:1 to 1:7, 1:1 to 1:6, 1:1 to 1:5, 1:1 to 1:4, 1:1 to 1:3, 1:1 to 1:2, 1:1 to 1:1.9, 1:1 to 1:1.8, 1:1 to 1:7, 1:1 to 1:1.6, 1:1 to 1:1.5, 1:1 to 1:1.4, 1:1 to 1:1.3, 1:1 to 1:1.2, 1:1 to 1:1.1, or 1:1 to 1:1.05.

In one embodiment, in transforming the host cell, the first plasmid and the second plasmid are in a molar ratio of 1:1, 1:1.05, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or 1:10.0.

In some embodiments, in transforming the host cell, the third plasmid and the fourth plasmid are in a molar ratio of 1:1 to 1:10, 1:1 to 1:9, 1:1 to 1:8, 1:1 to 1:7, 1:1 to 1:6, 1:1 to 1:5, 1:1 to 1:4, 1:1 to 1:3, 1:1 to 1:2, 1:1 to 1:1.9, 1:1 to 1:1.8, 1:1 to 1:7, 1:1 to 1:1.6, 1:1 to 1:1.5, 1:1 to 1:1.4, 1:1 to 1:1.3, 1:1 to 1:1.2, 1:1 to 1:1.1, or 1:1 to 1:1.05.

In one embodiment, in transforming the host cell, the third plasmid and the fourth plasmid are in a molar ratio of 1:1, 1:1.05, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or 1:10.0.

In some embodiments, in transforming the host cell, the first plasmid, the second plasmid, the third plasmid and the fourth plasmid are in a molar ratio of 1:(1-10):1:(1-10), e.g., 1:(1-5):1:(1-5), e.g., 2:3:2:3.

In some embodiments, in transforming the host cell, the first plasmid, the second plasmid, the third plasmid and the fourth plasmid are in a molar ratio of 1:(1-10):1:(1-10), 1:(1-9):1:(1-9), 1:(1-8):1:(1-8), 1:(1-7):1:(1-7), 1:(1-6):1:(1-6), 1:(1-5):1:(1-5), 1:(1-4):1:(1-4), 1:(1-3):1:(1-3) or 1:(1-2):1:(1-2).

In one embodiment, in transforming the host cell, the first plasmid, the second plasmid, the third plasmid and the fourth plasmid are in a molar ratio of 1:1:1:1, 1:1.05:1:1.05, 1:1.1:1:1.1, 1:1.2:1:1.2, 1:1.3:1:1.3, 1:1.4:1:1.4, 1:1.5:1:1.5 (or 2:3:2:3), 1:1.6:1:1.6, 1:1.7:1:1.7, 1:1.8:1:1.8, 1:1.9:1:1.9, 1:2.0:1:2.0, 1:2.1:1:2.1, 1:2.2:1:2.2, 1:2.3:1:2.3, 1:2.4:1:2.4, 1:2.5:1:2.5, 1:2.6:1:2.6, 1:2.7:1:2.7, 1:2.8:1:2.8, 1:2.9:1:2.9, 1:3.0:1:3.0, 1:3.1:1:3.1, 1:3.2:1:3.2, 1:3.3:1:3.3, 1:3.4:1:3.4, 1:3.5:1:3.5, 1:3.6:1:3.6, 1:3.7:1:3.7, 1:3.8:1:3.8, 1:3.9:1:3.9, 1:4.0:1:4.0, 1:4.1:1:4.1, 1:4.2:1:4.2, 1:4.3:1:4.3, 1:4.4:1:4.4, 1:4.5:1:4.5, 1:4.6:1:4.6, 1:4.7:1:4.7, 1:4.8:1:4.8, 1:4.9:1:4.9, 1:5.0:1:5.0, 1:5.1:1:5.1, 1:5.2:1:5.2, 1:5.3:1:5.3, 1:5.4:1:5.4, 1:5.5:1:5.5, 1:5.6:1:5.6, 1:5.7:1:5.7, 1:5.8:1:5.8, 1:5.9:1:5.9, 1:6.0:1:6.0, 1:6.1:1:6.1, 1:6.2:1:6.2, 1:6.3:1:6.3, 1:6.4:1:6.4, 1:6.5:1:6.5, 1:6.6:1:6.6, 1:6.7:1:6.7, 1:6.8:1:6.8, 1:6.9:1:6.9, 1:7.0:1:7.0, 1:7.1:1:7.1, 1:7.2:1:7.2, 1:7.3:1:7.3, 1:7.4:1:7.4, 1:7.5:1:7.5, 1:7.6:1:7.6, 1:7.7:1:7.7, 1:7.8:1:7.8, 1:7.9:1:7.9, 1:8.0:1:8.0, 1:8.1:1:8.1, 1:8.2:1:8.2, 1:8.3:1:8.3, 1:8.4:1:8.4, 1:8.5:1:8.5, 1:8.6:1:8.6, 1:8.7:1:8.7, 1:8.8:1:8.8, 1:8.9:1:8.9, 1:9.0:1:9.0, 1:9.1:1:9.1, 1:9.2:1:9.2, 1:9.3:1:9.3, 1:9.4:1:9.4, 1:9.5:1:9.5, 1:9.6:1:9.6, 1:9.7:1:9.7, 1:9.8:1:9.8, 1:9.9:1:9.9, or 1:10.0:1:10.0.

In some embodiments, the nucleic acid expression vector comprises:
a first plasmid comprising a nucleic acid molecule encoding the polypeptide H1 and a nucleic acid molecule encoding the polypeptide H2;
a second plasmid comprising a nucleic acid molecule encoding the polypeptide L1; and
a third plasmid comprising a nucleic acid molecule encoding the polypeptide L2.

In some embodiments, the nucleic acid expression vector comprises:
a first plasmid comprising a nucleic acid molecule encoding the heavy chain;
a second plasmid comprising a nucleic acid molecule encoding the first light chain; and
a third plasmid comprising a nucleic acid molecule encoding the second light chain.

In some embodiments, in transforming the host cell, the first plasmid, the second plasmid and the third plasmid are in a molar ratio of 1:(1-10):(1-10), preferably 1:(1-5):(1-5), more preferably 2:3:3.

In some embodiments, in transforming the host cell, the first plasmid, the second plasmid and the third plasmid are in a molar ratio of 1:(1-10):(1-10), 1:(1-9):(1-9), 1:(1-8):(1-8), 1:(1-7):(1-7), 1:(1-6):(1-6), 1:(1-5:(1-5), 1:(1-4):(1-4), 1:(1-3):(1-3) or 1:(1-2):(1-2).

In some embodiments, in transforming the host cell, the heavy chain plasmid, the first light chain plasmid and the second light chain plasmid are in a molar ratio of 1:1:1, 1:1.05:1.05, 1:1.1:1.1, 1:1.2:1.2, 1:1.3:1.3, 1:1.4:1.4, 1:1.5:1.5 (or 2:3:3), 1:1.6:1.6, 1:1.7:1.7, 1:1.8:1.8, 1:1.9:1.9, 1:2.0:2.0, 1:2.1:2.1, 1:2.2:2.2, 1:2.3:2.3, 1:2.4:2.4, 1:2.5:2.5, 1:2.6:2.6, 1:2.7:2.7, 1:2.8:2.8, 1:2.9:2.9, 1:3.0:3.0, 1:3.1:3.1, 1:3.2:3.2, 1:3.3:3.3, 1:3.4:3.4, 1:3.5:3.5, 1:3.6:3.6, 1:3.7:3.7, 1:3.8:3.8, 1:3.9:3.9, 1:4.0:4.0, 1:4.1:4.1, 1:4.2:4.2, 1:4.3:4.3, 1:4.4:4.4, 1:4.5:4.5, 1:4.6:4.6, 1:4.7:4.7, 1:4.8:4.8, 1:4.9:4.9, 1:5.0:5.0, 1:5.1:5.1, 1:5.2:5.2, 1:5.3:5.3, 1:5.4:5.4, 1:5.5:5.5, 1:5.6:5.6, 1:5.7:5.7, 1:5.8:5.8, 1:5.9:5.9, 1:6.0:6.0, 1:6.1:6.1, 1:6.2:6.2, 1:6.3:6.3, 1:6.4:6.4, 1:6.5:6.5, 1:6.6:6.6, 1:6.7:6.7, 1:6.8:6.8, 1:6.9:6.9, 1:7.0:7.0, 1:7.1:7.1, 1:7.2:7.2, 1:7.3:7.3, 1:7.4:7.4, 1:7.5:7.5, 1:7.6:7.6, 1:7.7:7.7, 1:7.8:7.8, 1:7.9:7.9, 1:8.0:8.0, 1:8.1:8.1, 1:8.2:8.2, 1:8.3:8.3, 1:8.4:8.4, 1:8.5:8.5, 1:8.6:8.6, 1:8.7:8.7, 1:8.8:8.8, 1:8.9:8.9, 1:9.0:9.0, 1:9.1:9.1, 1:9.2:9.2, 1:9.3:9.3, 1:9.4:9.4, 1:9.5:9.5, 1:9.6:9.6, 1:9.7:9.7, 1:9.8:9.8, 1:9.9:9.9, or 1:10.0:10.0.

In some embodiments, other methods known in the art may also be used in the present disclosure to balance the expression levels of the two heavy chains, such as the use of strong/weak promoters.

The present disclosure further provides a pharmaceutical composition comprising any one of the aforementioned antigen-binding proteins and a pharmaceutically acceptable carrier.

The pharmaceutically acceptable carrier refers to an ingredient, which is non-toxic to a subject, in a pharmaceutical formulation other than the active ingredient. The pharmaceutically acceptable carrier includes any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g., by injection or infusion).

In some other aspects, the present disclosure further provides a method for eliminating an immunosuppression-associated disease in a subject, the method comprising administering to the subject a therapeutically effective amount of the aforementioned antigen-binding protein or the aforementioned pharmaceutical composition; the therapeutically effective amount is a unit dose of the composition comprising 0.1-3000 mg of the aforementioned antigen-binding protein.

In some embodiments, the antigen-binding protein or pharmaceutical composition of the present disclosure is administered to the subject at a dose of about 10 μg/kg to about 1000 mg/kg in a single or cumulative application.

The present disclosure further provides use of any one of the aforementioned dimerized polypeptides or antigen-binding proteins in the preparation of a medicament.

The present disclosure further provides use of any one of the aforementioned dimerized polypeptides or antigen-binding proteins in the preparation of a medicament for treating a cancer, an autoimmune disease or an inflammatory disease.

The present disclosure further provides a method for treating and/or preventing a disease, such as a cancer, an autoimmune disease or an inflammatory disease, the method comprising administering to a patient in need thereof an effective amount of the aforementioned antigen-binding protein or pharmaceutical composition.

The present disclosure further provides any one of the aforementioned dimerized polypeptides, antigen-binding proteins or pharmaceutical compositions for use in treating a cancer, an autoimmune disease or an inflammatory disease.

In some embodiments, the cancer includes, but is not limited to, carcinoma, lymphoma, blastoma, sarcoma, leukemia and lymphoid malignancies. More specific examples of the cancer include squamous cell carcinoma, myeloma, small-cell lung cancer, non-small cell lung cancer (NSCLC), head and neck squamous cell carcinoma (HNSCC), neuroglioma, Hodgkin's lymphoma, non-Hodgkin's lymphoma, diffuse large B-cell lymphoma (DLBCL), follicular lymphoma, acute lymphoblastic leukemia (ALL), acute myeloid leukemia (AML), chronic lymphocytic leukemia (CLL), chronic myeloid leukemia (CML), primary mediastinal large B-cell lymphoma, mantle cell lymphoma (MCL), small lymphocytic lymphoma (SLL), large B-cell lymphoma rich in T-cells/histiocytes, multiple myeloma, myeloid cell leukemia-1 protein (Mc1-1), myelodysplastic syndrome (MDS), gastrointestinal (tract) cancer, kidney cancer, ovarian cancer, liver cancer, lymphoblastic leukemia, lymphocytic leukemia, colorectal cancer, endometrial cancer, prostate cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, bladder cancer, hepatoma, breast cancer, colon cancer, hepatocellular carcinoma (HCC), clear cell renal cell carcinoma (RCC), head and neck cancer, laryngeal cancer, hepatobiliary cancer, central nervous system cancer, esophageal cancer, malignant pleural mesothelioma, systemic light chain amyloidosis, lymphoplasmacytic lymphoma, myelodysplastic syndrome, myeloproliferative tumors, neuroendocrine tumors, Merkel cell carcinoma, testicular cancer and skin cancer.

In some embodiments, the autoimmune disease or the inflammatory disease is selected from the group consisting of: rheumatoid arthritis, psoriasis, Crohn's disease, ankylosing spondylitis, multiple sclerosis, type I diabetes, hepatitis, myocarditis, Sjogren's syndrome, autoimmune hemolytic anemia after transplant rejection, bullus pemphigoid, Graves' disease, Hashimoto's thyroiditis, systemic lupus erythematosus (SLE), myasthenia gravis, pemphigus and pernicious anemia.

### BRIEF DESCRIPTION OF THE DRAWINGS

FIG. 1 shows molecular weight deconvoluted mass spectra of the IdeS-digested PD-1 monoclonal antibody products in Example 3.

FIG. 2 shows the molecular formats in Example 4: 1+1 asymmetric bispecific antibodies, with one arm using natural CHUCK and the other arm using CH1/Ck comprising a non-natural disulfide bond.

FIGS. 3A-3D show molecular weight deconvoluted mass spectra of the papain-digested initial-purification products of bispecific antibodies in Example 4.

FIG. 4A shows a two-step purification chromatogram of the initial-purification product of the TJ030-PR1104 protein; FIG. 4B shows a deglycosylated intact molecular weight total ion current chromatogram (upper) and an ultraviolet spectrum (lower), as well as molecular assignments to major peaks, of the TJ030-PR1104 protein after polishing purification; FIG. 4C shows a deglycosylated reduced molecular weight total ion current chromatogram (upper) and an ultraviolet spectrum (lower), as well as molecular assignments to major peaks, of the TJ030-PR1104 protein after polishing purification.

FIG. 5 shows that 1+1 asymmetric PD-1×CTLA-4 bispecific antibodies contribute to the cross-linking of PD-1-expressing cells and CTLA-4-expressing cells.

FIG. 6 shows a schematic diagram of the molecular formats in Example 5: a 1+1 asymmetric bispecific antibody is shown, with one arm using natural CH1/κ and the other arm using CH1/Cλ, comprising a non-natural disulfide bond; or with one arm using CH1/κ comprising a non-natural disulfide bond and the other arm using natural CH1/Cλ.

FIG. 7A shows a deglycosylated intact molecular weight ultraviolet spectrum and molecular assignments to major peaks of TJ030-PR1313 after polishing purification; FIG. 7B shows a Fab molecular weight deconvoluted mass spectrum of Lys-C-digested TJ030-PR1313.

FIG. 8 shows molecular weight deconvoluted mass spectra of the products of GingisKHAN protease treatment of PSMA 1+1 biepitopic antibodies in which a non-natural disulfide bond is introduced into CH1/CL.

FIG. 9A shows a schematic diagram of the molecular formats of the FAP×CD40 2+2 symmetric bispecific antibodies of Example 8; FIG. 9B shows reduced molecular weight deconvoluted mass spectra of two FAP×CD40 antibodies; FIGS. 9C-9D show molecular weight deconvoluted mass spectra of two IdeS-digested FAP×CD40 antibodies.

FIG. 10A shows the FACS binding EC₅₀ results of FAP×CD40 antibodies to CD40; FIG. 10B shows the FACS binding EC₅₀ results of FAP×CD40 antibodies to FAP; FIGS. 10C and 10D show the activation activity results of FAP×CD40 antibodies for CD40 in the presence and absence of FAP.

### DETAILED DESCRIPTION

The term “antigen” refers to any substance that can induce an immune response in the body; examples of antigens include, but are not limited to, peptides, proteins, glycoproteins, polysaccharides, lipids and synthetic or naturally-occurring chemical compounds or combinations thereof.

The term “antigen-binding protein” refers to a protein capable of binding to an antigen, including but not limited to, full-length antibodies, antibody fragments or fusion proteins of antibodies and other polypeptides. The “binding” may be, for example, specific binding. Examples of antibody fragments include, but are not limited to (i) a Fab fragment, a monovalent fragment consisting of VL, VH, CL and CH1 domains; (ii) a F(ab')2 fragment, a bivalent fragment comprising two Fab fragments linked by a disulfide bridge in the hinge region; (iii) a Fd fragment, consisting of VH and CH1 domains; (iv) a Fv fragment, consisting of VH and VL domains of one arm of the antibody; (V) a dsFv, an antigen-binding fragment formed with VH and VL via interchain disulfide bonds therebetween; and (vi) a diabody, a bispecific antibody and a multispecific antibody, comprising such fragments as an scFv, a dsFv and a Fab. Furthermore, the two domains of the Fv fragment, VL and VH, are linked by a synthetic linker, so that they can generate a single protein chain in which the VL and VH regions are paired to form a monovalent molecule (referred to as single chain Fv (scFv); see, e.g., Bird et al., (1988) Science, 242:423-426; and Huston et al., (1988) Proc. Natl. Acad. Sci USA 85:5879-5883). Such single-chain antibodies are also included in the term “antibody fragment”. Such antibody fragments are obtained by conventional techniques known to those skilled in the art, and screened for utility in the same manner as for intact antibodies. Antigen-binding domains may be produced by a recombinant DNA technique or by enzyme catalysis or chemical cleavage of intact immunoglobulins.

The antibodies may be of different isotypes; for example, antibodies are divided into different types (e.g., 5 types: IgA, IgD, IgE, IgG and IgM, and further subtypes such as IgG1, IgG2, IgG3, IgG4, IgA1 and IgA2) according to the amino acid sequences of the heavy chain constant regions of the antibodies. Heavy chain constant regions corresponding to the above 5 types are referred to as α, δ, ϵ, γ and μ, respectively.

The light chain of an antibody can be considered either Kappa (κ) or Lamda (λ) based on its amino acid sequence.

The term “(light chain) CL region” refers to the constant region of an antibody light chain, which is a region well known in the related art. A CL region can be identified using a conventional method; for example, whether a region of interest is a CL region can be determined using its homology with a known antibody. The boundaries of a CL region may vary. The CL region in the human κ chain generally consists of 107 amino acid residues, and the CL region in the human λ chain generally consists of 106 amino acid residues. The natural cysteine in the CL region of the human κ chain is at position 214 according to the Kabat numbering scheme, and the natural cysteine in the CL region of the human 2 chain is at position 214 according to the Kabat numbering scheme.

The term “(heavy chain) CH1 region” refers to the first constant region of a heavy chain, which is a region known in the related art. A CH1 region as defined herein may also comprise part of the hinge region that follows the CH1 region (which may be comprised in the hinge region of the Fab region). A CH1 region can be identified using a conventional method; for example, whether a region of interest is a CH1 region can be determined using its homology with a known antibody. Since the boundaries of a CH1 region may vary, in the heavy chains of human IgG1, IgG2, IgG3 and IgG4, a CH1 region as defined herein generally consists of amino acid residues 118-215 and additional part of the hinge region (e.g., amino acid residues 216-224); in the heavy chain of IgM, a CH1 region as defined herein generally consists of amino acid residues 118-216, but is not limited thereto.

The term “Fc region” refers to a region corresponding to a fragment having no antigen binding ability among 2 types of fragments obtained when an antibody is cleaved with papain. Generally, a Fc region refers to the C-terminal region of an antibody heavy chain, which comprises part of the hinge region and the second constant (CH2) region and the third constant (CH3) region of the heavy chain. The boundaries of a heavy chain Fc region may vary; for example, the human IgG1 heavy chain Fc region consists of the amino acid residue of Thr225 to the carboxy terminus of the CH3 region.

The term “antibody-dependent cellular cytotoxicity (ADCC)” refers to a case in which secreted Ig bound to Fc receptors (FcRs) present on some cytotoxic cells (e.g., natural killer (NK) cells, neutrophils and macrophages) enable these cytotoxic effector cells to bind specifically to an antigen-bearing target cell and subsequently kill the target cell with cytotoxic agents. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess the ADCC activity of a molecule of interest, an in vitro ADCC assay, such as that described in U.S. Pat. No. 5,500,362 or can be performed. Useful effector cells for such assays include peripheral blood mononuclear cells (PBMCs) and natural killer (NK) cells. Alternatively, or additionally, the ADCC activity of the molecule of interest can be assessed in vivo, e.g., in an animal model such as that disclosed in Clynes et al., PNAS USA 95:652-656 (1998).

The term “Fc receptor” or “FcR” describes a receptor that binds to the Fc region of an antibody. A preferred FcR is a human FcR. In addition, a preferred FcR is a FcR that binds to an IgG antibody (a γ receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. The activating receptor FcγRIIA comprises an immunoreceptor tyrosine-based activation motif (ITAM) in the cytoplasmic domain thereof. The inhibiting receptor FcγRIIB comprises an immunoreceptor tyrosine-based inhibition motif (ITIM) in the cytoplasmic domain thereof (see review M. Daeron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “human effector cell” refers to leukocytes that express one or more FcRs and perform effector functions. Preferably, the cells express at least FcγRIII and perform ADCC effector function. Examples of human leukocytes that mediate ADCC include peripheral blood mononuclear cells (PBMCs), natural killer (NK) cells, monocytes, cytotoxic T cells, and neutrophils; PBMCs and NK cells are preferred. Effector cells can be isolated from a natural source, e.g., blood.

The term “complement-dependent cytotoxicity” or “CDC” refers to the lysis of a target cell in the presence of complement. Activation of the classical complement pathway is initiated by the binding of the first component of the complement system (Clq) to antibodies (of appropriate subclasses) that are bound to their cognate antigens. To assess complement activation, a CDC assay, e.g., as described in Gazzano-Santoro et al., J. Immunol. Methods 202:163 (1996), can be performed.

The term “therapeutically effective amount” refers to an amount of an antibody (including a multispecific antibody), an antigen-binding antibody fragment thereof or a derivative thereof for treating a disease or disorder in a subject. In the case of tumors (e.g., cancerous tumors), the therapeutically effective amount of the antibody or antibody fragment (e.g., a multispecific antibody or antibody fragment) may reduce the number of cancer cells, reduce the primary tumor size, inhibit (i.e., slow to some extent and preferably stop) cancer cell infiltration into peripheral organs, inhibit (i.e., slow to some extent and preferably stop) tumor metastasis, inhibit tumor growth to some extent, and/or relieve one or more of the symptoms associated with the disorder to some extent. To the extent that the antibody or antibody fragment or derivative thereof may prevent growth and/or kill existing cancer cells, it may be a cytostatic and/or cytotoxic agent. For cancer therapy, in vivo efficacy can be measured, for example, by assessing survival time, time to disease progression (TTP), response rate (RR), duration of response and/or quality of life.

The term “natural disulfide bond” refers to a cysteine-cysteine covalent bond that is generally present in wild-type polypeptides (antibodies, etc.). The term “non-natural disulfide bond” refers to a cysteine-cysteine covalent bond formed in a position other than the position of the “natural disulfide bond” described above.

The term “multispecific antibody” refers to an antibody that binds to two or more different epitopes (e.g., two, three, four or more different epitopes). The epitopes may be on the same antigen or different antigens. One example of a multispecific antibody is a “bispecific antibody” that binds to two different epitopes.

The term “valent” denotes the presence of a specified number of binding sites in an antibody molecule. A natural antibody, for example, has two binding sites and is bivalent. As such, the term “tetravalent” denotes the presence of four binding sites in an antibody molecule.

The term “amino acid” primarily refers to 20 naturally-occurring amino acids selected from the group consisting of: alanine (Ala or A), cysteine (Cys or C), aspartic acid (Asp or D), glutamic acid (Glu or E), phenylalanine (Phe or F), glycine (Gly or G), histidine (His or H), isoleucine (He or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W) and tyrosine (Tyr or Y). The term “amino acid residue” refers to the amino acid units in a polypeptide, because when the amino acids constituting the polypeptide bind to each other, some of their groups are involved in the formation of peptide bonds and a molecule of water is lost, i.e., the residues after the amino acids linked by peptide bonds lose water. The terms “amino acid” and “amino acid residue” are used interchangeably herein.

Whether an amino acid is “positively charged” or “negatively charged” is determined according to the electrical charge of the side chain of the amino acid as measured at pH 7.4. Amino acids can be grouped by common side chain properties: (1) hydrophobicity: norleucine, Met, Ala, Val, Leu and Ile; (2) neutral hydrophilicity: Cys, Ser, Thr, Asn and Gln; (3) acidity (negatively charged): Asp and Glu; (4) alkalinity (positively charged): His, Lys and Arg; (5) residues affecting chain orientation: Gly and Pro; and (6) aromaticity: Trp, Tyr and Phe.

The term “interface” refers to a surface where one or more amino acids in the first domain and one or more amino acids in the second domain from an antigen-binding protein or antibody interact or come into contact. Exemplary interfaces exist, for example, between CH1/CL, between VH/VL and/or between CH3/CH3. In some embodiments, the interface includes, for example, hydrogen bonds, electrostatic interactions or salt bridges between amino acids that form the interface.

The term “vector” refers to a nucleic acid molecule capable of transporting another nucleic acid to which it has been linked. In one embodiment, the vector is a “plasmid” that refers to a circular double-stranded DNA loop into which additional DNA segments may be ligated. In another embodiment, the vector is a viral vector, wherein additional DNA segments may be ligated into the viral genome. The vectors disclosed herein are capable of autonomous replication in a host cell into which they have been introduced (e.g., bacterial vectors having a bacterial origin of replication and episomal mammalian vectors) or being integrated into the genome of a host cell upon introduction into the host cell and thereby replicated along with the host genome (e.g., non-episomal mammalian vectors).

Methods for producing and purifying antibodies and antigen-binding fragments are well known in the art, for example, those described in chapters 5-8 and 15 of “Antibodies: A Laboratory Manual”, Cold Spring Harbor Press. For example, mice can be immunized with human PD-1 or a fragment thereof, and the obtained antibodies can be renatured and purified, and amino acid sequencing can be performed by conventional methods. Likewise, antigen-binding fragments can be prepared by conventional methods. The antibody or the antigen-binding fragment described herein is genetically engineered to contain one or more additional human 1Rs in the non-human CDRs. Human FR germline sequences can be obtained at the website http://imgt.cines.fr of ImMunoGeneTics (IMGT) or from the immunoglobulin journal, 2001ISBN012441351, by comparing the IMGT human antibody variable region germline gene database with the MOE software.

The term “host cell” refers to a cell into which an expression vector has been introduced. Host cells may include bacterial, microbial, plant or animal cells. Bacteria susceptible to transformation include members of the Enterobacteriaceae family, such as strains of Escherichia coli or Salmonella; members of the Bacillaceae family, such as Bacillus subtilis; Pneumococcus; Streptococcus and Haemophilus influenzae. Suitable microorganisms include Saccharomyces cerevisiae and Pichia pastoris. Suitable animal host cell lines include CHO (Chinese hamster ovary cell line) and NS0 cells.

The engineered antibody or the antigen-binding fragment of the present disclosure can be prepared and purified by conventional methods. For example, cDNA sequences encoding the heavy and light chains can be cloned and recombined into a GS expression vector. Recombinant immunoglobulin expression vectors can be stably transfected into CHO cells. As a more recommended prior art, mammalian expression systems will result in glycosylation of antibodies, particularly at the highly conserved N-terminal site of the Fc region. Stable clones were obtained by expressing antibodies that bind specifically to human PD-1, or antibodies that bind to both PD-1 and PD-L1. Positive clones are expanded in a serum-free medium of a bioreactor to produce antibodies. The culture medium with the secreted antibody can be purified by conventional techniques. For example, purification is performed using an A or G Sepharose FF column containing an adjusted buffer. Non-specifically bound fractions are washed away. The bound antibody is eluted by the pH gradient method, and the antibody fragments are detected by SDS-PAGE and collected. The antibody can be filtered and concentrated by conventional methods. Soluble mixtures and polymers can also be removed by conventional methods, such as molecular sieves and ion exchange. The resulting product needs to be immediately frozen, e.g., at −70° C., or lyophilized

Unless otherwise indicated, the terms “first” and “second” of the present disclosure are merely generic identifiers, and should not be construed as identifying specific or particular portions of the antigen-binding protein provided herein; the “first” and “second” in any embodiment of the present disclosure can be reversed; for example, any amino acid substitutions described in the present disclosure as being in the first CH1 and the first CL may alternatively be in the second CH1 and the second CL.

The sequences involved in the present disclosure are as follows:

SEQ ID NO: 1
(PD-1/HC)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPS
NTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGN
VFSCSVMHEALHNHYTQKSLSLSPGK
SEQ ID NO: 2
(PD-1/LC)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
SEQ ID NO: 3
(PD-1/HC; S131C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPCSKSTSGGTAALGCLVKDYFP
EPVTVSWNSGALTSGVHTFPAVLQSSGLYSLSSVVTVPSSSLGTQTYICNVNHKP
SNTKVDKKVEPKSADKTHTCPPCPAPEAAGGPSVFLFPPKPKDTLMISRTPEVTC
VVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQG
NVFSCSVMHEALHNHYTQKSLSLSPGK
SEQ ID NO: 4
(PD-1/HC; L128C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPCAPSSKSTSGGTAALGCLVKDYFPE
PVTVSWNSGALTSGVHTFPAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGN
VFSCSVMHEALHNHYTQKSLSLSPGK
SEQ ID NO: 5
(PD-1/HC; A129C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLCPSSKSTSGGTAALGCLVKDYFPE
PVTVSWNSGALTSGVHTFPAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGN
VFSCSVMHEALHNHYTQKSLSLSPGK
SEQ ID NO: 6
(PD-1/HC; F170C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGTAALGCLVKDYFPE
PVTVSWNSGALTSGVHTCPAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGN
VFSCSVMHEALHNHYTQKSLSLSPGK
SEQ ID NO: 7
(PD-1/HC; A141C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGTACLGCLVKDYFPE
PVTVSWNSGALTSGVHTFPAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGN
VFSCSVMHEALHNHYTQKSLSLSPGK
SEQ ID NO: 8
(PD-1/LC; P119C is underlined)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPSVFIFCPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EA
SEQ ID NO: 9
(PD-1/LC; S121C is underlined)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPSVFIFPPCDEQLKSGTASVVCLLNNFYPREAKVQWKVDNALQSGN
SQESVTEQDSKDSTYSLSSTLTLSKADYEKHKVYACEVTHQGLSSPVTKSFNRG
EA
SEQ ID NO: 10
(PD-1/LC; T164C is underlined)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPSVFIFPPSDEQLKSGTASVVCLLNNFYPREAKVQWKVDNALQSGN
SQESVCEQDSKDSTYSLSSTLTLSKADYEKHKVYACEVTHQGLSSPVTKSFNRG
EA
SEQ ID NO: 11
(PD-1/LC; L135C is underlined)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPSVFIFPPSDEQLKSGTASVVCCLNNFYPREAKVQWKVDNALQSGN
SQESVTEQDSKDSTYSLSSTLTLSKADYEKHKVYACEVTHQGLSSPVTKSFNRG
EA
SEQ ID NO: 12
(PD-1/HC)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPS
NTKVDKKVEPKSCDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRWQQGN
VFSCSVMHEALHNHYTQKSLSLSPGK
SEQ ID NO: 13
(PD-1/LC)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
SEQ ID NO: 14
(CTLA-4/HC)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKK
VEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 15
(CTLA-4/LC)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKRTV
AAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 16
(CTLA-4/HC; F126C is underlined)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVCPLAPSSKSTSGGTAALGCLVKDYFPEPVTVSWN
SGALTSGVHTFPAVLQSSGLYSLSSVVTVPSSSLGTQTYICNVNHKPSNTKVDKK
VEPKSADKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 17
(CTLA-4/LC; S121C is underlined)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKRTV
AAPSVFIFPPCDEQLKSGTASVVCLLNNFYPREAKVQWKVDNALQSGNSQESV
TEQDSKDSTYSLSSTLTLSKADYEKHKVYACEVTHQGLSSPVTKSFNRGEA
SEQ ID NO: 18
(CTLA-4/HC; L128C is underlined)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPCAPSSKSTSGGTAALGCLVKDYFPEPVTVSWN
SGALTSGVHTFPAVLQSSGLYSLSSVVTVPSSSLGTQTYICNVNHKPSNTKVDKK
VEPKSADKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 19
(CTLA-4/HC; F170C is underlined)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPLAPSSKSTSGGTAALGCLVKDYFPEPVTVSWN
SGALTSGVHTCPAVLQSSGLYSLSSVVTVPSSSLGTQTYICNVNHKPSNTKVDKK
VEPKSADKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 20
(CTLA-4/LC; T164C is underlined)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKRTV
AAPSVFIFPPSDEQLKSGTASVVCLLNNFYPREAKVQWKVDNALQSGNSQESVC
EQDSKDSTYSLSSTLTLSKADYEKHKVYACEVTHQGLSSPVTKSFNRGEA
SEQ ID NO: 21
(CTLA-4/HC; S131C is underlined)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPLAPCSKSTSGGTAALGCLVKDYFPEPVTVSWN
SGALTSGVHTFPAVLQSSGLYSLSSVVTVPSSSLGTQTYICNVNHKPSNTKVDKK
VEPKSADKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 22
(CTLA-4/LC; P119C is underlined)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKRTV
AAPSVFIFCPSDEQLKSGTASVVCLLNNFYPREAKVQWKVDNALQSGNSQESV
TEQDSKDSTYSLSSTLTLSKADYEKHKVYACEVTHQGLSSPVTKSFNRGEA
SEQ ID NO: 23
(PD-1/HC; F126C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVCPLAPSSKSTSGGTAALGCLVKDYFP
EPVTVSWNSGALTSGVHTFPAVLQSSGLYSLSSVVTVPSSSLGTQTYICNVNHKP
SNTKVDKKVEPKSADKTHTCPPCPAPEAAGGPSVFLFPPKPKDTLMISRTPEVTC
VVVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQVSL
WCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 24
(PD-1/HC; L128C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPCAPSSKSTSGGTAALGCLVKDYFPE
PVTVSWNSGALTSGVHTFPAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRWQQGN
VFSCSVMHEALHNHYTQKSLSLSPGK
SEQ ID NO: 25
(PD-1/HC; F170C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGTAALGCLVKDYFPE
PVTVSWNSGALTSGVHTCPAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRWQQGN
VFSCSVMHEALHNHYTQKSLSLSPGK
SEQ ID NO: 26
(PD-1/HC; S131C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPCSKSTSGGTAALGCLVKDYFP
EPVTVSWNSGALTSGVHTFPAVLQSSGLYSLSSVVTVPSSSLGTQTYICNVNHKP
SNTKVDKKVEPKSADKTHTCPPCPAPEAAGGPSVFLFPPKPKDTLMISRTPEVTC
VVVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQVSL
WCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 27
(CTLA-4/LC)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKGQP
KAAPSVTLFPPSSEELQANKATLVCLISDFYPGAVTVAWKADSSPVKAGVETTTP
SKQSNNKYAASSYLSLTPEQWKSHRSYSCQVTHEGSTVEKTVAPTECS
SEQ ID NO: 28
(CTLA-4/HC; P171C is underlined)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPLAPSSKSTSGGTAALGCLVKDYFPEPVTVSWN
SGALTSGVHTFCAVLQSSGLYSLSSVVTVPSSSLGTQTYICNVNHKPSNTKVDKK
VEPKSADKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 29
(CTLA-4/LC; S165C is underlined)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKGQP
KAAPSVTLFPPSSEELQANKATLVCLISDFYPGAVTVAWKADSSPVKAGVETTTP
CKQSNNKYAASSYLSLTPEQWKSHRSYSCQVTHEGSTVEKTVAPTEAS
SEQ ID NO: 30
(PD-1/LC)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKGQPKAAPSVTLFPPSSEELQANKATLVCLISDFYPGAVTVAWKADSSPVKAG
VETTTPSKQSNNKYAASSYLSLTPEQWKSHRSYSCQVTHEGSTVEKTVAPTECS
SEQ ID NO: 31
(PD-1/HC; P171C is underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGTAALGCLVKDYFPE
PVTVSWNSGALTSGVHTFCAVLQSSGLYSLSSVVTVPSSSLGTQTYICNVNHKPS
NTKVDKKVEPKSADKTHTCPPCPAPEAAGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRWQQGN
VFSCSVMHEALHNHYTQKSLSLSPGK
SEQ ID NO: 32
(PD-1/LC; S165C is underlined)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKGQPKAAPSVTLFPPSSEELQANKATLVCLISDFYPGAVTVAWKADSSPVKAG
VETTTPCKQSNNKYAASSYLSLTPEQWKSHRSYSCQVTHEGSTVEKTVAPTEAS
SEQ ID NO: 33
(PD-1/HC; T139R and F170C are underlined)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGRAALGCLVKDYFP
EPVTVSWNSGALTSGVHTCPAVLQSSGLYSLSSVVTVPSSSLGTQTYICNVNHKP
SNTKVDKKVEPKSADKTHTCPPCPAPEAAGGPSVFLFPPKPKDTLMISRTPEVTC
VVVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQVSL
WCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 34
(PD-1/LC; S114E and T164C are underlined)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPEVFIFPPSDEQLKSGTASVVCLLNNFYPREAKVQWKVDNALQSGN
SQESVCEQDSKDSTYSLSSTLTLSKADYEKHKVYACEVTHQGLSSPVTKSFNRG
EA
SEQ ID NO: 35
(CTLA-4/HC; T139D is underlined)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPLAPSSKSTSGGDAALGCLVKDYFPEPVTVSWN
SGALTSGVHTFPAVLQSSGLYSLSSVVTVPSSSLGTQTYICNVNHKPSNTKVDKK
VEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 36
(CTLA-4/LC; S114K is underlined)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKRTV
AAPKVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
SEQ ID NO: 37
(PD-1/HC)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGRAALGCLVKDYFP
EPVTVSWNSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQVSL
WCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 38
(PD-1/LC)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIKRTVAAPEVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
SEQ ID NO: 39
(PD-1/HC)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSSASTKGPSVFPLAPSSKSTSGGDAALGCLVKDYFP
EPVTVSWNSGALTSGVHTFPAVLQSSGLYSLSSVVTVPSSSLGTQTYICNVNHKP
SNTKVDKKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQVSL
WCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 40
(PD-1/LC)
DIVMTQTPLSLPVTPGEPASISCRSSQSLVHSTGNTYLEWYLQKPGQSPQLLIYK
VSNRFSGVPDRESGSGSGTDFTLKISRVEAEDVGVYYCFQGSHVPYTFGGGTKV
EIKRTVAAPKVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
SEQ ID NO: 41
(CTLA-4/HC)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSSASTKGPSVFPLAPSSKSTSGGRAALGCLVKDYFPEPVTVSWN
SGALTSGVHTFPAVLQSSGLYSLSSVVTVPSSSLGTQTYICNVNHKPSNTKVDKK
VEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAVKGFYPS
DIAVEWESNGQPENNYKTTPPVLDSDGSFFLVSKLTVDKSRWQQGNVFSCSVM
HEALHNHYTQKSLSLSPGK
SEQ ID NO: 42
(CTLA-4/LC)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIKRTV
AAPEVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 43
(PD-1/HCDR1)
DYEMH
SEQ ID NO: 44
(PD-1/HCDR2)
LIDPETGGTVYNQKFKD
SEQ ID NO: 45
(PD-1/HCDR3)
ERFSYYGSTSDWYFD
SEQ ID NO: 46
(PD-1/LCDR1)
RSSQSLVHSTGNTYLE
SEQ ID NO: 47
(PD-1/LCDR2)
KVSNRFS
SEQ ID NO: 48
(PD-1/LCDR3)
FQGSHVPYT
SEQ ID NO: 49
(PD-1/VH)
EVQLVQSGAEVKKPGSSVKVSCKASGGTFSDYEMHWVRQAPGQGLEWMGLID
PETGGTVYNQKFKDRVTITADKSTSTAYMELSSLRSEDTAVYYCARERFSYYGS
TSDWYFDVWGQGTTVTVSS
SEQ ID NO: 50
(PD-1/VL)
DIVMTQTPLSLPVTPGEPASISCRSSQSLVHSTGNTYLEWYLQKPGQSPQLLIYK
VSNRFSGVPDRFSGSGSGTDFTLKISRVEAEDVGVYYCFQGSHVPYTFGGGTKV
EIK
SEQ ID NO: 51
(CTLA-4/HCDR1)
SYTMH
SEQ ID NO: 52
(CTLA-4/HCDR2)
FISYDGNNKYYADSVKG
SEQ ID NO: 53
(CTLA-4/HCDR3)
TGWLGPFDY
SEQ ID NO: 54
(CTLA-4/LCDR1)
RASQSVGSSYLA
SEQ ID NO: 55
(CTLA-4/LCDR2)
GAFSRAT
SEQ ID NO: 56
(CTLA-4/LCDR3)
QQYGSSPWT
SEQ ID NO: 57
(CTLA-4/VH)
QVQLVESGGGVVQPGRSLRLSCAASGFTFSSYTMHWVRQAPGKGLEWVTFISY
DGNNKYYADSVKGRFTISRDNSKNTLYLQMNSLRAEDTAIYYCARTGWLGPFD
YWGQGTLVTVSS
SEQ ID NO: 58
(CTLA-4/VL)
EIVLTQSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKVEIK

### EXAMPLES

The following examples further illustrate the present disclosure, but the present disclosure is not limited thereto. The experimental methods in the examples of the present disclosure in which specific conditions are not specified are generally performed under conventional conditions such as Antibodies: A Laboratory Manual and Molecular Cloning: A Laboratory Manual by Cold Spring Harbor Laboratory, or under conditions recommended by the manufacturer of the raw material or the goods. Reagents without specific origins indicated are commercially available conventional reagents.

### Example 1

### Experimental Materials and Methods

### 1.1. Monoclonal Antibody Expression and Purification

CHO-S cells (Thermo, A29133) in good growth states and in the logarithmic growth phase were used. The cells were centrifuged and 250 mL of the cells was inoculated at 6×10⁶ cells/mL. Solution 2 (800 μL of transfection reagent was diluted and well mixed with 9.2 mL of culture medium) was added to solution 1 (250 μg of plasmids was diluted and well mixed with 10 mL of culture medium), making a total volume of 20 mL. The solutions were gently and well mixed and then incubated at room temperature for 1-5 mM The mixed transfection solution was added dropwise to the cell culture with shaking. The culture flask was then placed on a 5% CO₂, 32° C. shaker. After 18-22 h of culture, 16 mL of Feed (Thermo, A29133) and 0.6 mL of Enhancer (Thermo, A29133) were added. On day 5, 16 mL of Feed (Thermo, A29133) was added. The cells were cultured at 120 rpm at 32° C. with 5% CO₂ until days 12-14 and centrifuged, and the supernatant was collected and purified by affinity chromatography (MabSelect SuRe column, GE, 17-5438).

### 1.2. Bispecific Antibody Expression and Purification

Bispecific antibodies were expressed in the same way as the monoclonal antibody PD-1 but were purified using a slightly more complicated strategy than the monoclonal antibody: the affinity chromatography initial purification in the first step was similar to that of the monoclonal antibody, but ion exchange chromatography was needed sometimes for polishing purification. Different anion and cation exchange chromatography methods can be selected according to the isoelectric point properties of antibodies.

The anion exchange chromatography method is as follows: the one-step purified sample was loaded onto a HiTrap Q HP column (GE, 17515601), equilibrated with solution A (20 mM PB, pH 7.0), and then subjected to gradient elution with 0-100% solution B (20 mM PB, 1 M NaCl, pH 7.0). The cation exchange chromatography method is as follows: the one-step purified sample was loaded onto a Capto S ImpAct pre-packed column (GE, 17-5441-22), equilibrated with solution A (50 mM NaAc, 50 mM NaCl, pH 5.0), and then subjected to gradient elution with 0-100% solution B (50 mM NaAc, 500 mM NaCl, pH 5.0).

### 1.3. Mass Spectrometry Analysis

In the present disclosure, protein samples were biologically analyzed using conventional high-resolution mass spectrometers 6530B ESI-Q-TOF (Agilent) and XEVO G2-XS Q-Tof (Waters).

### 1.3.1. Intact Molecular Weight

Samples were diluted and then separated by reversed-phase chromatography and analyzed by high-resolution mass spectrometry to give original spectra with different mass-to-charge ratios. After processing using deconvolution software, the intact molecular weights of the antibodies were obtained. Specifically, 50 μg of sample and standard were taken, diluted with mobile phase A (0.1% formic acid in water) to 0.5 mg/mL and centrifuged at 4° C. at 12,000 rpm for 10 mM, and the supernatants were transferred to sampler vials. Before sample injection, the chromatography column (Waters, 186008946) was equilibrated with 95% mobile phase A until it was stable. After sample injection, gradient elution was performed using mobile phase A and mobile phase B (0.1% formic acid in acetonitrile). After sample collection was complete, corresponding mass spectrum data were obtained at the position where the target peak appeared.

### 1.3.2. Deglycosylated Intact Molecular Weight

Samples were diluted and then separated by reversed-phase chromatography and analyzed by high-resolution mass spectrometry to give original spectra with different mass-to-charge ratios. After processing using deconvolution software, the intact molecular weights of the deglycosylated antibodies were obtained. Specifically, 100 μg of test sample and standard were taken, 2 μL of peptide N-glycosidase F (PNGase F, BioLabs, P0704L) was added to each of them, and 50 mM ammonium bicarbonate solution was added to bring the volume to 100 μL; deglycosylation was performed at 37° C. for 3 h. After incubation was complete, proteins were diluted with mobile phase A to a concentration of 0.5 μg/μL and centrifuged at 4° C. at 12,000 rpm for 10 mM, and the supernatants were transferred to sampler vials. Before sample injection, the chromatography column was equilibrated with 95% mobile phase A until it was stable. After sample injection, gradient elution was performed using mobile phase A and mobile phase B (0.1% formic acid in acetonitrile). After sample collection was complete, corresponding mass spectrum data were obtained at the position where the target peak appeared.

### 1.3.3. Reduced Molecular Weight

Samples were diluted and then separated by reversed-phase chromatography and analyzed by high-resolution mass spectrometry to give original spectra with different mass-to-charge ratios. After processing using deconvolution software, the reduced molecular weights of the antibodies were obtained. Specifically, 100 μg of test sample and standard were taken, 50 mM ammonium bicarbonate solution was added to bring the volume to 90 μL, and 10 μL of DTT was added to make a final concentration of 10 mM; the mixtures were incubated at 37° C. for 30 mM. After the incubation was complete, proteins were diluted with mobile phase A to a concentration of μg/μL and centrifuged at 4° C. at 12,000 rpm for 10 mM, and the supernatants were transferred to sampler vials. Before sample injection, the chromatography column was equilibrated with 95% mobile phase A until it was stable. After sample injection, gradient elution was performed using mobile phase A and mobile phase B (0.1% formic acid in acetonitrile). After sample collection was complete, corresponding mass spectrum data were obtained at the position where the target peak appeared.

### 1.3.4. Molecular Weight of F(ab′)2 After IdeS Digestion

Samples were digested with immunoglobulin G-degrading enzyme (IdeS, Promega, v7511) to give Fab fragments. The fragments were separated by reversed-phase chromatography and analyzed by high-resolution mass spectrometry to give original spectra with different mass-to-charge ratios. After processing using deconvolution software, the molecular weights of the antibody F(ab′)₂ fragments were obtained, and pairing information was obtained from the molecular weights. Specifically, 100 μg of test sample and standard were taken and diluted to 0.5 μg/μL with 50 mM Tris-HCl (pH 7.50); 100 μL of each of the dilutions was taken, 1 μL of IdeS was added, and the mixtures were incubated at 37° C. for 30 mM. After the reactions were complete, 1 μL of 10% aqueous formic acid solution was added, and the supernatants were transferred to sampler vials. Before sample injection, the chromatography column was equilibrated with 95% mobile phase A until it was stable. After sample injection, gradient elution was performed using mobile phase A and mobile phase B (0.1% formic acid in acetonitrile). After sample collection was complete, corresponding mass spectrum data were obtained at the position where the target peak appeared.

1.3.5. Molecular Weight of Fab after Lys-C or Papain Digestion

Samples were digested with protease (Lys-C, RHINO BIO, QIP-004-A or Papain, Solarbio, G8430) to give Fab fragments. The fragments were separated by reversed-phase chromatography and analyzed by high-resolution mass spectrometry to give original spectra with different mass-to-charge ratios. After processing using deconvolution software, the molecular weights of the antibody Fab fragments were obtained, and pairing information was obtained from the molecular weights. Take Lys-C digestion for Fab molecular weight measurement as an example: 100 μg of test sample and standard were taken and diluted to 0.5 μg/μL with 50 mM Tris-HCl (pH 7.50); 100 μL of each of the dilutions was taken, 0.25 μg of Lys-C was added, and the mixtures were incubated at 37° C. for 5 mM After the reactions were complete, 1 μL of 10% aqueous formic acid solution was added, and the supernatants were transferred to sampler vials. Before sample injection, the chromatography column was equilibrated with 95% mobile phase A until it was stable. After sample injection, gradient elution was performed using mobile phase A and mobile phase B (0.1% formic acid in acetonitrile). After sample collection was complete, corresponding mass spectrum data were obtained at the position where the target peak appeared.

### 1.3.6. Free Sulfhydryl Group Analysis

To obtain information about the sites and proportion of free sulfhydryl groups of test sample: 250 μg of test sample was taken, 95 μL of 8 M guanidine hydrochloride solution was added, and the mixture was incubated at 56° C. for 40 min. After heating was complete, 5μL of 0.1 M maleimide (NEM) was added. The mixture was well mixed and then reacted at room temperature in a dark place for 35 min. The mixture was centrifuged at 13,000 rpm for 15 mM, 100 μL of 50 mM Tris-HCl was added, and the centrifugation was continued; this process was repeated 3 times. Then 90 μL of 50 mM Tris-HCl and 10 μL of 1 M DTT solution were added, and the mixture was reacted for 40 min. The mixture was centrifuged at 13,000 rpm for 15 mM, 100 μL of 50 mM Tris-HCl was added, and the centrifugation was continued; this process was repeated 3 times. 20 μL of 1 M iodoacetamide (IAM) was added. The mixture was well mixed and then reacted at room temperature in a dark place for 35 min. The mixture was centrifuged at 13,000 rpm for 15 mM, 100 μL of 50 mM Tris-HCl was added, and the centrifugation was continued; this process was repeated 3 times. Trypsin was then added to make a ratio of enzyme to test sample of 1:25 (w/w), and the mixture was incubated at 37° C. for 16 h. After the mixture was taken out, 1.0 μL of formic acid was added to stop the reaction. Mass spectrometry analysis was performed, and the data were analyzed.

### 1.4. Cross-Linking of Cells

The ability of PD-1×CTLA-4 bispecific antibodies to co-bind to cells highly expressing human PD-1 and human CTLA-4 after polishing purification was determined by flow cytometry. First, HEK293 cells were transiently transfected with human CTLA-4 plasmids, and 24 h after the transfection, the HEK293 cells highly expressing CTLA-4 were labeled with Cell Trace Far red (Invitrogen, C34564), and the CHO-K1/PD-1 stably transfected strain was labeled with Cell Trace Violet (Invitrogen, C34557). The cells were added to a 96-well U-bottom plate (Costar, 3599) at 2E5 cells/well, and the test antibody was diluted to 100 nM, 10 nM, 1 nM, 0.1 nM and 0.01 nM and added to the 96-well U-bottom plate at 50 μL/well, making a total volume of 150 μL/well. The plate was incubated at 4° C. in a dark place for 1 h. The double-positive cell percentage of HEK293/CTLA-4 of CTLA-4 and CHO-K1/PD-1 was determined by flow cytometry.

### 1.5. Cell-Binding FACS Assays of FAP and CD40 Arms

CHO cells stably expressing human FAP (i.e., CHO/FAP cells) and HEK293 cells 48 hours after transient transfection (i.e., HEK293/CD40 cells) in the logarithmic growth phase were collected by centrifugation, washed with PBS and then centrifuged. The cells were plated at 2E5 cells/100 μL/well and centrifuged at 400 g for 5 min Different concentrations of test antibody were added. The cells were incubated on ice for 1 h, washed with PBS and centrifuged at 400 g for 5 min The goat anti-human secondary antibody Alexa Fluor 488 with a fluorophore was added, and the cells were stained in an ice bath for 1 h, washed twice with PBS and then assayed on a flow cytometer.

### 1.6. Effect of FAP×CD40 Bispecific Antibodies on CD40 Signaling Pathway Activation

To verify the effect of FAP×CD40 bispecific antibodies on CD40 signaling pathway activation and the effect on CD40 signaling pathway activation in the presence of FAP, the positive cell strain highly expressing human CD40, HEK-Blue CD40L cell, and the Flp-In CHO cell strain stably expressing human FAP were used. The cells were diluted to 5.5E5/mL with DMEM/F12K medium containing 10% heat-inactivated serum, and 90 μL of HEK-Blu CD40L cell suspension was added to each well of a 96-well flat-bottom cell culture plate, along with 90 μL of medium or the Flp-In CHO/FAP cell strain. To each well was added 20 μL of serially diluted antibody. The group without the antibody was used as a negative control. The plate was incubated overnight in a 37° C., 5% CO2 incubator. To another 96-well flat-bottom cell culture plate were added 180 μL of Quanti-Blue assay reagent and 20 μL of cell culture supernatant. The plate was incubated at room temperature for 30 min and OD655 readings were then taken on a microplate reader.

### Example 2

### Introduction of Amino Acid Substitutions in CH1/CL Interaction Interface

Positions for the introduction of non-natural disulfide bonds were designed and are shown in Table 1.

TABLE 1
Positions for the introduction of non-natural disulfide bonds into CH1/CL
Amino acid mutations for the introduction of non-natural disulfide bonds

| | |
|---|---|
| IgG1 CH1 mutation position¹ | CL mutation position² |

Antibodies comprising Kappa CL

| | | |
|---|---|---|
| Non-natural disulfide bond pairing mode #1 | S131C | P119C |
| Non-natural disulfide bond pairing mode #2 | L128C | S121C |
| Non-natural disulfide bond pairing mode #3 | A129C | S121C |
| Non-natural disulfide bond pairing mode #4 | F170C | T164C |
| Non-natural disulfide bond pairing mode #5 | A141C | L135C |

Antibodies comprising Lambda CL

| | | |
|---|---|---|
| Non-natural disulfide bond pairing mode #6 | P171C | S165C |

Amino acid mutations for the removal of naturally-occurring
disulfide bonds in heavy and light chains of antibodies

| | IgG1 CH1 mutation position¹ | CL mutation position² |
|---|---|---|
| Antibodies comprising Kappa CL | C220A | C214A |
| Antibodies comprising Lambda CL | C220A | C214A |

¹EU numbering
²Kabat numbering

Positions of amino acid mutations for the introduction of electrostatic effects were also designed and are shown in Table 2.

TABLE 2
Positions for the introduction of electrostatic effects into CH1/CL
Amino acid mutations for the introduction of electrostatic effects

| | IgG1 CH1 | CL mutation |
|---|---|---|
| | mutation position¹ | position² |
| Electrostatic effect pairing mode #1 | T139R | S114E |
| Electrostatic effect pairing mode #2 | T139R | S114D |
| Electrostatic effect pairing mode #3 | T139K | S114E |
| Electrostatic effect pairing mode #4 | T139K | S114D |
| Electrostatic effect pairing mode #5 | T139D | S114K |
| Electrostatic effect pairing mode #6 | T139D | S114R |
| Electrostatic effect pairing mode #7 | T139E | S114K |
| Electrostatic effect pairing mode #8 | T139E | S114R |

¹EU numbering
²Kabat numbering

### Example 3

### Construction of Expression Vector for PD-1 Monoclonal Antibodies and Protein Expression and Purification

Nucleic acids encoding the heavy chain (set forth in SEQ ID NO: 1) and the light chain (set forth in SEQ ID NO: 2) of the PD-1-IgG1-LALA antibody were each constructed onto a pTT5 plasmid vector. On the basis of this, a C220A mutation (EU numbering) was introduced into the heavy chain, and a C214A mutation (Kabat numbering) was introduced into the light chain. The two mutations were introduced simultaneously, completely eliminating the interchain disulfide bond naturally occurring at these positions (position 220 of CH1 and position 214 of CL).

To express PD-1 monoclonal antibodies with non-natural disulfide bond linking, S131C (SEQ ID NO: 3), L128C (SEQ ID NO: 4), A129C (SEQ ID NO: 5) or F170C (SEQ ID NO: 6) was further introduced into the heavy chains from which natural disulfide bonds were removed; similarly, P119C (SEQ ID NO: 8), S121C (SEQ ID NO: 9) or T164C (SEQ ID NO: 10) was further introduced into the light chains from which natural disulfide bonds were removed, as shown in Table 3.

TABLE 3
The light and heavy chain sequences and plasmid ratios of PD-1
monoclonal antibodies comprising non-natural disulfide bonds

| Protein No. | |
|---|---|
| (heavy chain:light chain | PD-1 arm |

| plasmid molar ratio) | Heavy chain | Light chain |
|---|---|---|
| TJ021-PR0103 | SEQ ID NO: 3 | SEQ ID NO: 8 |
| (2:3) | (S131C) | (P119C) |
| TJ021-PR0104 | SEQ ID NO: 4 | SEQ ID NO: 9 |
| (2:3) | (L128C) | (S121C) |
| TJ021-PR0105 | SEQ ID NO: 5 | SEQ ID NO: 9 |
| (2:3) | (A129C) | (S121C) |
| TJ021-PR0108 | SEQ ID NO: 6 | SEQ ID NO: 10 |
| (2:3) | (F170C) | (T164C) |

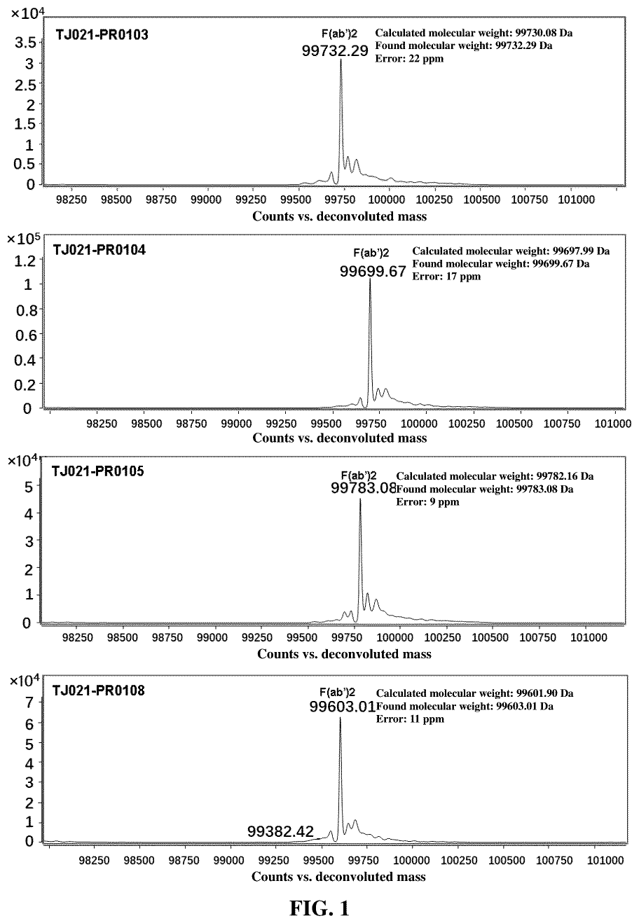

According to the sequences and plasmid ratios of PD-1 monoclonal antibodies shown in Table 3, the antibodies were expressed and purified according to the methods of Examples 1.1 and 1.2, and the PD-1 antibodies after introduction of non-natural disulfide bonds and one-step purification had similar protein expression levels and purity to PD-1 antibodies comprising natural disulfide bonds: there was no significant difference. To confirm whether non-natural disulfide bonds were formed, corresponding PD-1 antibodies were digested with IdeS to give molecular fragments of F(ab′)2 (FIG. 1), which demonstrate that the introduction of cysteine at the specific positions described above, S131C-P119C, L128C-S121C, A129C-S121C and F170C-T164C, enabled the formation of CH1/CL interchain disulfide bonds.

To further confirm the formation of non-natural disulfide bonds, particularly the presence of unpaired free cysteine residues in antibody molecules, the free sulfhydryl groups of monoclonal antibodies were further quantitatively characterized according to the method of Example 1.3. We defined: proportion of free sulfhydryl groups (%)=mass spectrometry signal intensity of NEM-modified peptide fragments/total mass spectrometry signal intensity of peptide fragments ×100%. The results show that the overall proportion of free sulfhydryl groups in PD-1 monoclonal antibodies with non-natural disulfide bonds S131C-P119C, L128C- S 121C, A129C-S121C and F170C-T164C is <3%, which indicates that these introduced non-natural cysteine residues can be paired to form disulfide bonds.

### Example 4

### PD-1×CTLA-4 Bispecific Antibodies with Introduction of Non-Natural Disulfide Bonds into CH1/Cκ

### 4.1. Molecular Formats

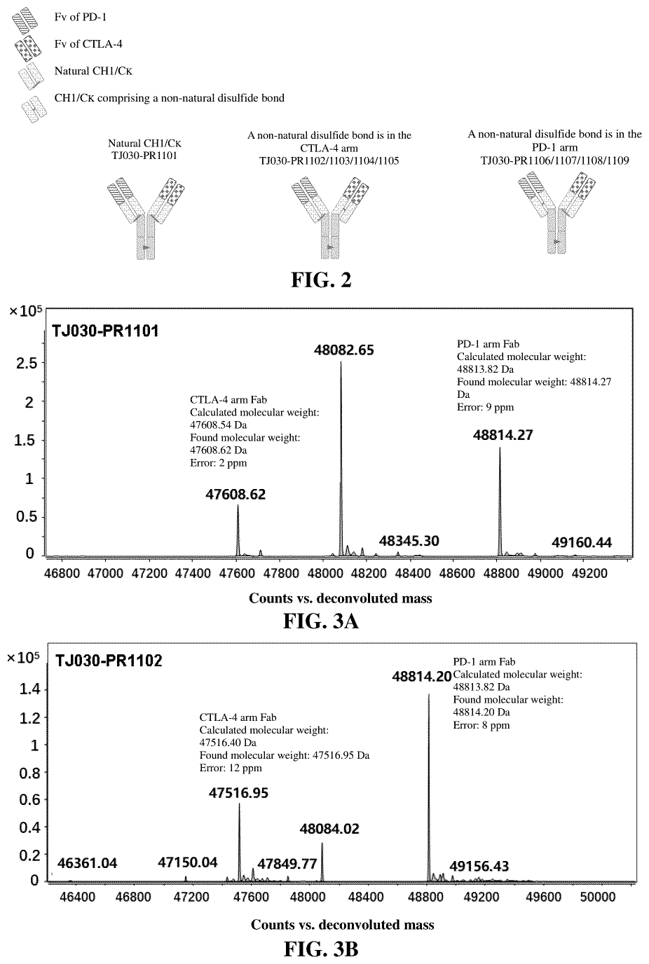

In view of the excellent performance of non-natural disulfide bonds applied to PD-1 monoclonal antibodies, 1+1 asymmetric bispecific antibodies were further designed on the basis of KIH (S354C/T366W; Y349C/T366S/L368A/Y407V). Theoretically, a non-natural disulfide bond can be used on either the PD-1 arm or the CTLA-4 arm; the Fc of the PD-1 arm comprises a cavity (hole) and the Fc of the CTLA-4 arm comprises a protuberance (knob), and vice versa; four combinations can thus be formed. In this example, the Fc of the PD-1 arm was made to comprise the amino acid mutation T366W leading to formation of a protuberance (knob), and the Fc of the CTLA-4 arm was made to comprise the amino acid mutation T366S/L368A/Y407V leading to formation of a cavity (hole) (a schematic diagram of the structure is shown in FIG. 2).

According to the sequences and plasmid ratios of PD-1×CTLA-4 bispecific antibodies shown in Table 4, bispecific antibodies were expressed and purified using the methods of Examples 1.1 and 1.2.

TABLE 4
The sequences and plasmid ratios of PD-1 × CTLA-4
bispecific antibodies comprising non-natural disulfide bonds

| Protein No. | | | | |
|---|---|---|---|---|
| (PD-1 heavy chain:PD-1 | | | | |
| light chain:CTLA-4 heavy | | | | |
| chain:CTLA-4 light chain | PD-1 arm | | CTLA-4 arm | |
| plasmid molar ratio) | Heavy chain | Light chain | Heavy chain | Light chain |
| TJ030-PR1101 | SEQ ID NO: 12 | SEQ ID NO: 13 | SEQ ID NO: 14 | SEQ ID NO: 15 |
| (1:1:1:1) | | | | |
| TJ030-PR1102 | SEQ ID NO: 12 | SEQ ID NO: 13 | SEQ ID NO: 16 | SEQ ID NO: 17 |
| (1:1:1:1) | | | | |
| TJ030-PR1103 | SEQ ID NO: 12 | SEQ ID NO: 13 | SEQ ID NO: 18 | SEQ ID NO: 17 |
| (1:1:1:1) | | | | |
| TJ030-PR1104 | SEQ ID NO: 12 | SEQ ID NO: 13 | SEQ ID NO: 19 | SEQ ID NO: 20 |
| (1:1:1:1) | | | | |
| TJ030-PR1105 | SEQ ID NO: 12 | SEQ ID NO: 13 | SEQ ID NO: 21 | SEQ ID NO: 22 |
| (1:1:1:1) | | | | |
| TJ030-PR1106 | SEQ ID NO: 23 | SEQ ID NO: 9 | SEQ ID NO: 14 | SEQ ID NO: 15 |
| (1:1:1:1) | | | | |
| TJ030-PR1107 | SEQ ID NO: 24 | SEQ ID NO: 9 | SEQ ID NO: 14 | SEQ ID NO: 15 |
| (1:1:1:1) | | | | |
| TJ030-PR1108 | SEQ ID NO: 25 | SEQ ID NO: 10 | SEQ ID NO: 14 | SEQ ID NO: 15 |
| (1:1:1:1) | | | | |
| TJ030-PR1109 | SEQ ID NO: 26 | SEQ ID NO: 8 | SEQ ID NO: 14 | SEQ ID NO: 15 |
| (1:1:1:1) | | | | |

### 4.2. Mass Spectrometry Mispairing Analysis of Initial-Purification Products

### 4.2.1. Effect of Non-Natural Disulfide Bond on Expression of Corresponding Fab

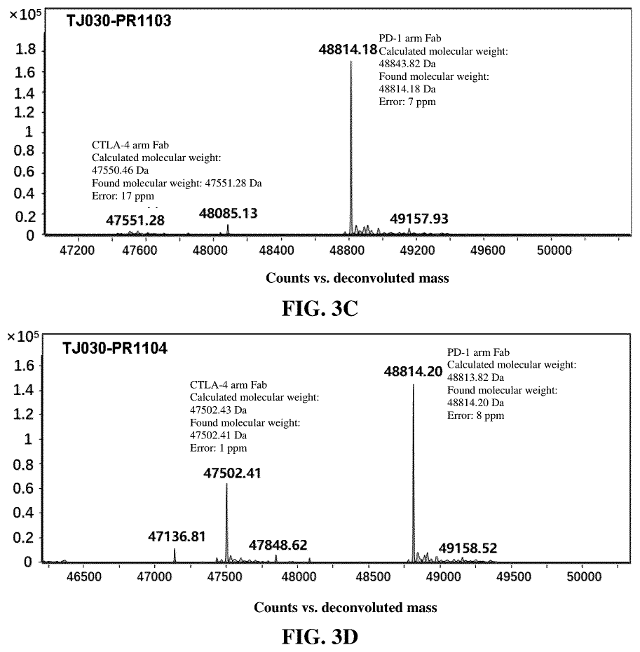

Take PD-1×CTLA-4 bispecific antibodies as an example: in the case of TJ030-PR1103 where a non-natural disulfide bond L128C-S121C was introduced into the CTLA-4 arm and which retained a natural disulfide bond in the PD-1 arm, in the co-transfection and expression of plasmids encoding 4 chains, the expression level of the CTLA-4 arm was significantly reduced by competition from the PD-1 arm. The molecular weight deconvoluted mass spectrometry results of the papain-digested initial-purification products of bispecific antibodies (FIG. 3) show that for TJ030-PR1101, peak intensity of CTLA-4 arm/peak intensity of PD-1 arm=1:2; for TJ030-PR1103, peak intensity of CTLA-4 arm/peak intensity of PD-1=1:46. In comparison, TJ030-PR1104 into which a non-natural disulfide bond F170C-T164C was introduced and TJ030-PR1105 into which a non-natural disulfide bond S131C-P119C was introduced did not have reduced expression levels in the presence of competition from the natural disulfide bond Fab arm: the peak intensity of CTLA-4 arm/peak intensity of PD-1 arm was kept to about 1:2.

### 4.2.2. Proportion of Light Chain Mispairing

To further quantify light chain mispairing, the proportion of correct pairing was calculated according to the molecular weight deconvoluted mass spectrometry results in FIG. 3; the calculation formula is: (peak intensity of correct PD-1 arm+peak intensity of correct CTLA-4 arm)/(peak intensity of correct PD-1 arm+peak intensity of correct CTLA-4 arm+peak intensity of other impurities). The results are shown in Table 5. It can be seen that whether the non-natural disulfide bonds were placed into the CTLA-4 arm or the PD-1 arm, the proportion of correct pairing in TJ030-PR1103 (L128C-S121C) and TJ030-PR1104 (F170C-T164C) was greatly improved relative to TJ030-PR1101 (using a natural disulfide bond) and to TJ030-PR1102 (F126C-S121C) reported in the prior art. For TJ030-PR1105 (S131C-P119C), when the non-natural disulfide bond was placed into the PD-1 arm, the proportion of correct pairing was greatly improved relative to both TJ030-PR1101 (using a natural disulfide bond) and TJ030-PR1102 (F126C-S121C) reported in the prior art.

TABLE 5
The proportion of correct pairing in PD-1 × CTLA-4 bispecific antibodies
after introduction of non-natural disulfide bonds into CH1/Cκ

| | Place a non-natural disulfide bond into | | Place a non-natural disulfide bond into | |
|---|---|---|---|---|
| | the CTLA-4 arm | | the PD-1 arm | |
| | | Proportion of | | Proportion of |
| | | correct pairing | | correct pairing |
| | Name of protein | (%) | Name of protein | (%) |
| Natural disulfide | TJ030-PR1101 | 47.7 | TJ030-PR1101 | 47.7 |
| bond | | | | |
| F126C-S121C | TJ030-PR1102 | 86.8 | TJ030-PR1106 | 66.3 |
| L128C-S121C | TJ030-PR1103 | 94.5 | TJ030-PR1107 | 68.9 |
| F170C-T164C | TJ030-PR1104 | 92.6 | TJ030-PR1108 | 76.5 |
| S131C-P119C | TJ030-PR1105 | 41.7 | TJ030-PR1109 | 77.5 |

### 4.4. Mass Spectrometry Mispairing Analysis of Polishing-Purification Products

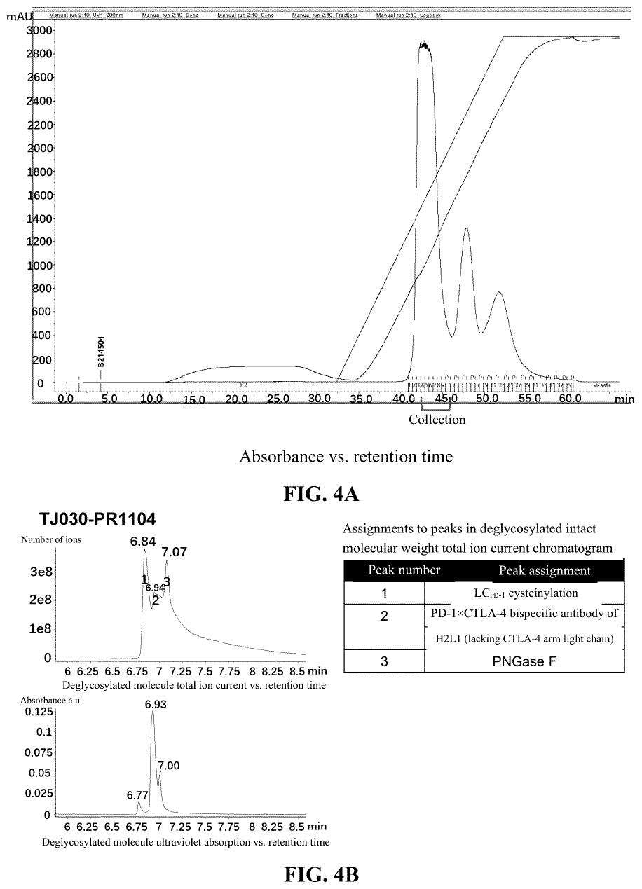

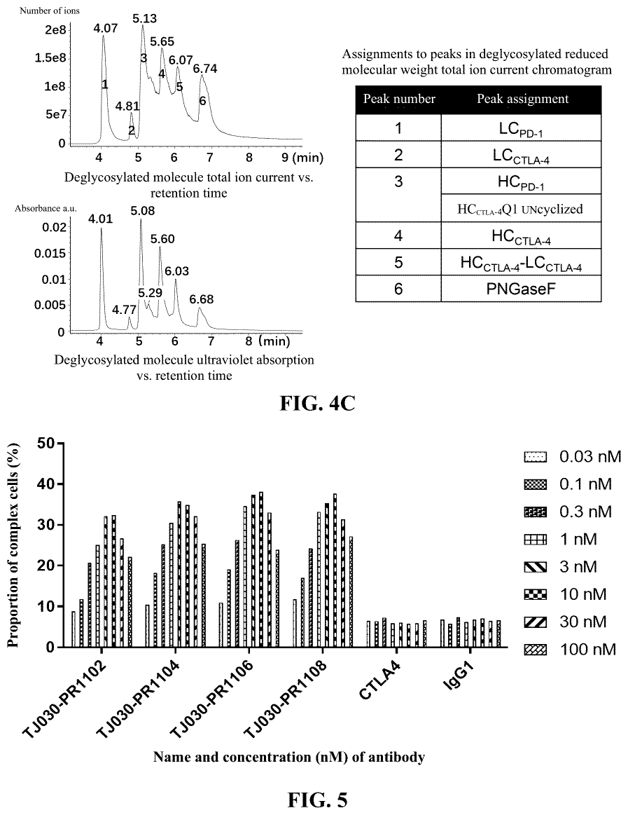

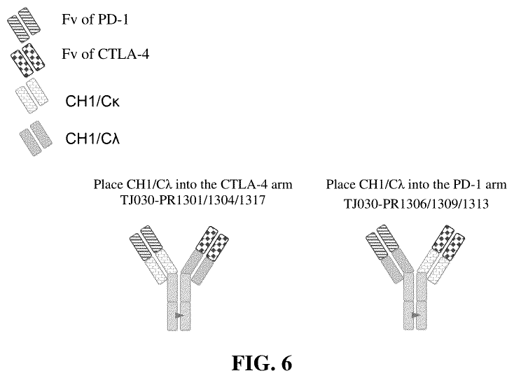

Take TJ030-PR1104 as an example: after the one-step purification product was purified through a polishing step according to the method of Example 1.2, characteristic peaks were collected for subsequent analysis, as shown in FIG. 4A.

### 4.4.1. Intact Molecular Weight and Deglycosylated Intact Molecular Weight

Determination of deglycosylated intact molecular weight was performed using the method of 1.3.2; the results are shown in FIG. 4B. The deglycosylated intact molecular weight spectrum shows the protein of interest, a 1+1 asymmetric bispecific antibody with correct pairing, and also shows the formation of H2L1 (in a format comprising two heavy chains and one light chain and lacking a CTLA-4 arm light chain) as a byproduct and the formation of a PD-1 light chain cysteine conjugate (LC_{PD-1}−Cys). The reason for this may be that the expression level of the CTLA-4 arm, particularly the light chain of the CTLA-4 arm, was insufficient, leading to the production of a large amount of H2L1. We speculate that such an incomplete H2L1 antibody molecule requires additional LC_{PD-1} to further stabilize the structure; even so, there were still no detectable light chain mispairing products of TJ030-PR1104 after polishing purification.

### 4.4.2. Determination of Reduced Molecular Weight and Deglycosylated Reduced Molecular Weight

Determination of reduced molecular weight and deglycosylated reduced molecular weight was performed using the method of 1.3.3; the results are shown in FIG. 4C. In addition to the 4 reduced protein sequences corresponding to the bispecific antibody TJ030-PR1104, the reduced molecular weight and deglycosylated reduced molecular weight mass spectra also show a pairing of CTLA-4 heavy chain and/or light chain (HC_{CTLA-4}-LC_{CTLA-4}), which further confirms the correct formation of the F170C-T164C non-natural disulfide bond.

### 4.5. Cross-Linking of Cells

According to Example 1.4, the ability of each of the PD-1×CTLA-4 bispecific antibodies TJ030-PR1102 (F126C-S121C was placed into the CTLA-4 arm), TJ030-PR1104 (F170C-T164C was placed into the CTLA-4 arm), TJ030-PR1106 (F126C-S121C was placed into the PD-1 arm) and TJ030-PR1108 (F170C-T164C was placed into the PD-1 arm) to co-bind to cells highly expressing human PD-1 and human CTLA-4 after polishing purification was determined by flow cytometry, with CTLA-4 monoclonal antibody and IgG as negative controls. The cross-linking results show that the 1+1 asymmetric PD-1×CTLA-4 bispecific antibodies can cross-link a cell expressing PD-1 and a cell expressing CTLA-4 (Table 6 and FIG. 5) and that the proportion of double-positive cells produced by cross-linking of cells gradually increased with the increasing bispecific antibody concentration within the range of 0.03 nM to 10 nM. Such cross-linking of cells occurred in bispecific antibody molecule incubation only rather than monoclonal antibody or IgG1 incubation. In addition, the bispecific antibody molecules TJ030-PR1104 and TJ030-PR1108 with introduction of the non-natural disulfide bond pair F170C-T164C resulted in significantly more double-positive cells at each concentration point in 0.03 nM-100 nM than the reported TJ030-PR1102 with a non-natural disulfide bond F126C-S121C.

TABLE 6
The proportion of double-positive cells as a function of concentration in the cross-linking of cells

| | 0.03 nM | 0.1 nM | 0.3 nM | 1 nM | 3 nM | 10 nM | 30 nM | 100 nM |
|---|---|---|---|---|---|---|---|---|
| TJ030-PR1102 | 8.873% | 11.826% | 20.721% | 25.081% | 32.191% | 32.384% | 26.741% | 22.157% |
| TJ030-PR1104 | 10.432% | 18.246% | 25.261% | 30.468% | 35.784% | 34.834% | 32.122% | 25.394% |
| TJ030-PR1106 | 10.932% | 19.097% | 26.322% | 34.658% | 37.428% | 38.135% | 33.056% | 23.863% |
| TJ030-PR1108 | 11.765% | 17.012% | 24.248% | 33.171% | 35.334% | 37.704% | 31.394% | 27.168% |
| CTLA4 | 6.444% | 6.41% | 7.28% | 5.834% | 6.05% | 5.755% | 5.874% | 6.616% |
| IgG1 | 6.741% | 5.791% | 7.404% | 6.247% | 6.78% | 7.102% | 6.462% | 6.65% |

### Example 5

### Optimization of Plasmid Ratio in PD-1×CTLA-4 Bispecific Antibody Expression

Bispecific antibody expression was performed using the method of Example 1.1. During transient transfection and expression, we increased the plasmid ratio for the light chain (the plasmid ratio of heavy chain to light chain was changed from 1:1 to 2:3), in hope of decreasing the corresponding proportion of antibodies with a “two heavy chains and one light chain (H2L1)” configuration. In addition, we introduced CH1/Ck or a non-natural disulfide bond mutant thereof into one arm and CH1/Cλ, or a non-natural disulfide bond mutant thereof into the other arm of the 1+1 asymmetric bispecific antibodies, so as to subsequently use Kappa Select or Lambda Select for purification and to study the effect of light chain subtype choices on reduction in light chain mispairing. Table 7 shows the sequences and plasmid ratios of PD-1×CTLA-4 bispecific antibodies.

TABLE 7
The sequences plasmid ratios of PD-1 × CTLA-4 bispecific antibodies

| Protein No. | | | | |
|---|---|---|---|---|
| (PD-1 heavy | | | | |
| chain:PD-1 light | | | | |
| chain:CTLA-4 heavy | | | | |
| chain:CTLA-4 light | | | | |
| chain plasmid molar | PD-1 arm | | CTLA-4 arm | |
| ratio) | Heavy chain | Light chain | Heavy chain | Light chain |
| TJ030-PR1301 | SEQ ID NO: 12 | SEQ ID NO: 13 | SEQ ID NO: 14 | SEQ ID NO: 27 |
| (2:3:2:3) | | | | |
| TJ030-PR1304 | SEQ ID NO: 12 | SEQ ID NO: 13 | SEQ ID NO: 28 | SEQ ID NO: 29 |
| (2:3:2:3) | | | | |
| TJ030-PR1317 | SEQ ID NO: 25 | SEQ ID NO: 10 | SEQ ID NO: 14 | SEQ ID NO: 27 |
| (2:3:2:3) | | | | |
| TJ030-PR1306 | SEQ ID NO: 12 | SEQ ID NO: 30 | SEQ ID NO: 14 | SEQ ID NO: 15 |
| (2:3:2:3) | | | | |
| TJ030-PR1309 | SEQ ID NO: 31 | SEQ ID NO: 32 | SEQ ID NO: 14 | SEQ ID NO: 15 |
| (2:3:2:3) | | | | |
| TJ030-PR1313 | SEQ ID NO: 12 | SEQ ID NO: 30 | SEQ ID NO: 19 | SEQ ID NO: 20 |
| (2:3:2:3) | | | | |
| TJ030-PR1230 | SEQ ID NO: 33 | SEQ ID NO: 34 | SEQ ID NO: 35 | SEQ ID NO: 36 |
| (2:3:2:3) | | | | |
| TJ030-PR1231 | SEQ ID NO: 25 | SEQ ID NO: 10 | SEQ ID NO: 14 | SEQ ID NO: 15 |
| (2:3:2:3) | | | | |
| TJ030-PR1320 | SEQ ID NO: 12 | SEQ ID NO: 13 | SEQ ID NO: 14 | SEQ ID NO: 15 |
| (2:3:2:3) | | | | |
| TJ030-PR1321 | SEQ ID NO: 37 | SEQ ID NO: 38 | SEQ ID NO: 35 | SEQ ID NO: 36 |
| (2:3:2:3) | | | | |
| TJ030-PR1322 | SEQ ID NO: 39 | SEQ ID NO: 40 | SEQ ID NO: 41 | SEQ ID NO: 42 |
| (2:3:2:3) | | | | |

PD-1×CTLA-4 bispecific antibodies were purified through a polishing step according to the method of Example 1 and analyzed by mass spectrometry. As shown in Table 8, after non-natural disulfide bonds P171C-S165C (TJ030-PR1304 and TJ030-PR1309) and F170C-T164C (TJ030-PR1317 and TJ030-PR1313) were introduced into the CH1/CL interfaces of PD-1×CTLA-4 bispecific antibodies, the proportion of correct pairing was significantly increased relative to bispecific antibodies TJ030-PR1301 and TJ030-PR1306 using natural disulfide bonds, whether the non-natural disulfide bonds were placed into the CTLA-4 arm or the PD-1 arm.

TABLE 8
The proportion of correct pairing in PD-1 × CTLA-4 bispecific
antibodies after introduction of non-natural disulfide bonds into CH1/CL

| | Place CH1/Cλ into | | Place CH1/Cλ into | |
|---|---|---|---|---|
| | the CTLA-4 arm | | the PD-1 arm | |
| | | Proportion | | Proportion |
| | | of correct | | of correct |
| | Name of | pairing | Name of | pairing |
| | protein | (%) | protein | (%) |
| Natural | TJ030-PR1301 | 63.0 | TJ030-PR1306 | 79.5* |
| disulfide bond | | | | |
| P171C-S165C | TJ030-PR1304 | 100 | TJ030-PR1309 | 87.9 |
| F170C-T164C | TJ030-PR1317 | 100 | TJ030-PR1313 | 100 |

*Correct pairing and mispairing lead to very similar (<2 Da) Fab molecular weights and it is thus difficult to distinguish between them.

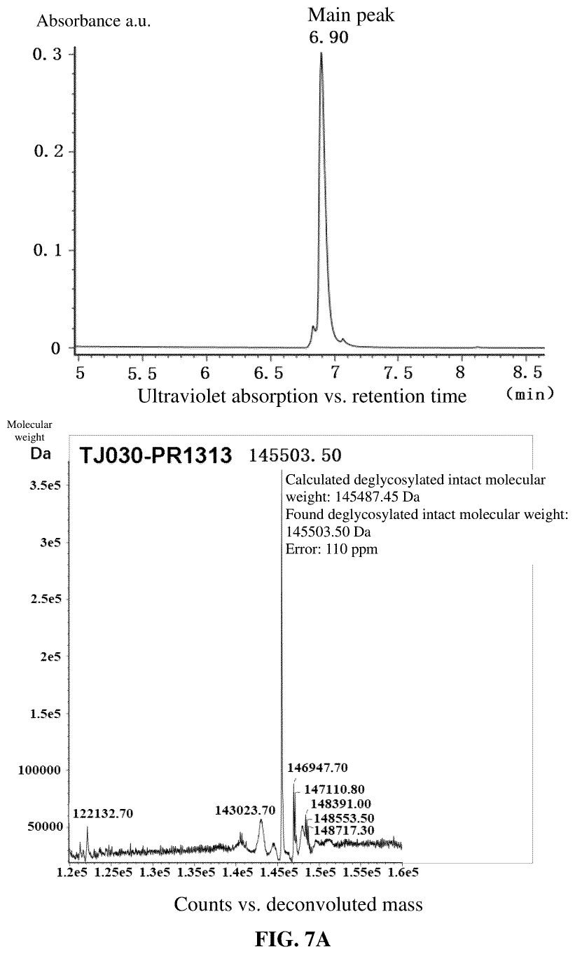

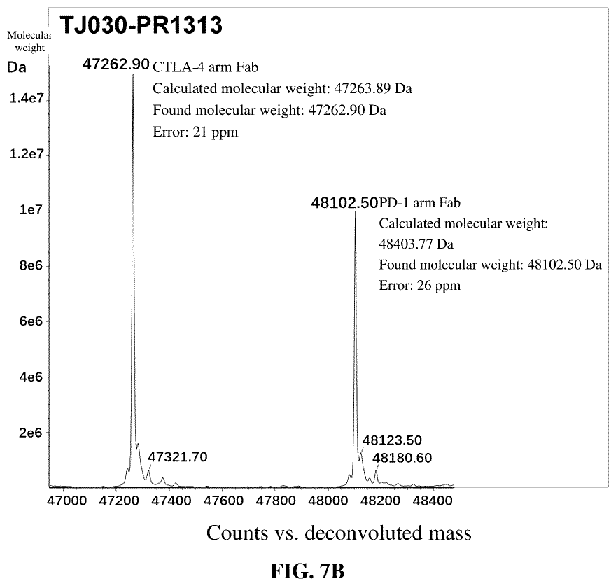

Take TJ030-PR1313 as an example: the bispecific antibody after Lys-C digestion and cation exchange polishing purification demonstrated that Fab pairing was completely correct and no light chain mispairing occurred. The deglycosylated intact molecular weight met expectations, and no obvious H2L1 byproduct lacking a light chain was present (FIG. 7).

As shown in Table 9, both TJ030-PR1231 and TJ030-PR1317 comprise amino acid mutations F170C-T164C and light chains of κ subtype in their PD-1 arms; the only difference between them is that TJ030-PR1231 comprises a light chain of κ subtype in its CTLA-4 arm and TJ030-PR1317 comprises a light chain of λ subtype in its CTLA-4 arm: TJ030-PR1317 shows a higher proportion of correct pairing; that is, using different light chain subtypes in the two arms is beneficial to improving the proportion of correct pairing in a bispecific antibody.

TABLE 9
The effect of light chain subtype choices in PD-1 × CTLA-4
bispecific antibodies on the proportion of correct pairing

| | | Proportion of |
|---|---|---|
| Name of | | correct |
| protein | Light chain subtypes | pairing (%) |
| TJ030-PR1231 | Place CH1/Ck into the PD-1 arm | 89.8 |
| | Place CH1/Ck into the CTLA-4 arm | |
| TJ030-PR1317 | Place CH1/Ck into the PD-1 arm | 100 |
| | Place CH1/Cλ into the CTLA-4 arm | |

### Example 6

### Introduction of Electrostatic Effects Reduces Light Chain Mispairing

The sequences and plasmid ratios of the bispecific antibody molecules TJ030-PR1220, TJ030-PR1221 and TJ030-PR1222 after introduction of electrostatic effect mutations at position 139 of CH1 and position 114 of CL are shown in Table 7. The results show that introduction of electrostatic effects can reduce light chain mispairing.

After electrostatic effect mutations were introduced at position 139 of CH1 and position 114 of CL in the PD-1 arm and the CTLA-4 arm of TJ030-PR1231, the bispecific antibody molecule TJ030-PR1230 was obtained (the sequences and plasmid ratio are shown in Table 7). The one-step initial-purification product shows no light chain mispairing (Table 10), which demonstrates that the electrostatic effect of HC139-LC114 can further reduce light chain mispairing.

TABLE 10
The proportion of correct pairing in PD-1 × CTLA-4 bispecific
antibodies after introduction of electrostatic effects into CH1/CL

| | Proportion | | | | |
|---|---|---|---|---|---|
| | of correct | CTLA-4 arm | | PD-1 arm | |
| | pairing | Heavy | Light | Heavy | Light |
| | (%) | chain | chain | chain | chain |
| TJ030-PR1231 | 89.8 | T139 | S114 | T139 | S114 |
| (2:3:2:3) | | | | | |
| TJ030-PR1230 | 100.0 | T139R | S114E | T139D | S114K |
| (2:3:2:3) | | | | | |

In addition, it can be seen from Table 5 that comparison of TJ030-PR1230 with TJ030-PR1108 shows that increasing the transfection ratio of light chain (particularly the light chain CTLA-4 arm with weak expression) can also reduce light chain mispairing.

### Example 7

### PSMA 1+1 Biepitopic Antibodies with Introduction of Non-Natural Disulfide Bonds into CH1/CL

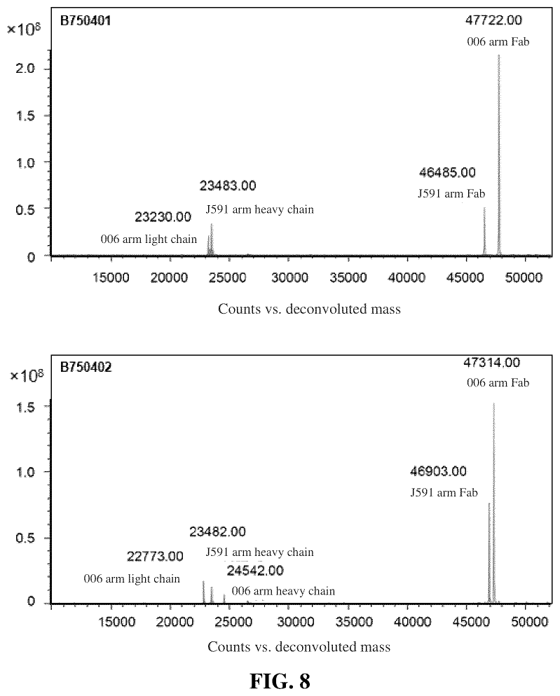

PSMA 1+1 asymmetric biepitopic antibodies were constructed according to the antibody sequences and plasmid ratios shown in Table 11, 1+1 asymmetric bispecific antibodies of heavy chain heterodimerization (T366W; T366S/L368A/Y407V) were also achieved using the KiH method, and the ProteinA initial-purification products were analyzed by mass spectrometry. No light chain mispairing products were found in the products of GingisKHAN protease treatment (FIG. 8).

TABLE 11
The sequences and plasmid ratios of
PSMA 1 + 1 biepitopic antibodies

| Protein No. | | | | |
|---|---|---|---|---|
| (J591 heavy | | | | |
| chain:J591 light | | | | |
| chain:006 heavy | J591 arm | | 006 arm | |
| chain:006 light chain | Heavy | Light | Heavy | Light |
| plasmid molar ratio) | chain | chain | chain | chain |
| B750401 (2:3:2:3) | SEQ ID | SEQ ID | SEQ ID | SEQ ID |
| | NO: 59 | NO: 60 | NO: 61 | NO: 62 |
| B750402 (2:3:2:3) | SEQ ID | SEQ ID | SEQ ID | SEQ ID |
| | NO: 63 | NO: 64 | NO: 65 | NO: 66 |

SEQ ID NO: 59
(J591/HC)
EVQLVQSGPEVKKPGATVKISCKTSGYTFTEYTIHWVKQAPGKGLEWIGNINPN
NGGTTYNQKFEDKATLTVDKSTDTAYMELSSLRSEDTAVYYCAAGWNFDYWG
QGTLLTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVS
NKALPAPIEKTISKAKGQPREPQVYTLPPSREEMTKNQVSLWCLVKGFYPSDIAV
EWESNGQPENNYKTTPPVLDSDGSFFLYSKLTVDKSRWQQGNVFSCSVMHEAL
HNHYTQKSLSLSPGK
SEQ ID NO: 60
(J591/LC)
DIQMTQSPSSLSTSVGDRVTLTCKASQDVGTAVDWYQQKPGPSPKLLIYWASTR
HTGIPSRFSGSGSGTDFTLTISSLQPEDFADYYCQQYNSYPLTFGPGTKVDIKGQP
KANPTVTLFPPSSEELQANKATLVCLISDFYPGAVTVAWKADGSPVKAGVETTK
PSKQSNNKYAASSYLSLTPEQWKSHRSYSCQVTHEGSTVEKTVAPTECS
SEQ ID NO: 61
(006/HC; F170C is underlined)
QVQLVESGGGVVQPGRSLRLSCAASGFAFSRYGMHWVRQAPGKGLEWVAVIW
YDGSNKYYADSVKGRFTISRDNSKNTQYLQMNSLRAEDTAVYYCARGGDFLYY
YYYGMDVWGQGTTVTVSSASTKGPSVFPLAPSSKSTSGGTAALGCLVKDYFPE
PVTVSWNSGALTSGVHTCPAVLQSSGLYSLSSVVTVPSSSLGTQTYICNVNHKPS
NTKVDKKVEPKSADKTHTCPPCPAPELLGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLSC
AVKGFYPSDIAVEWESNGQPENNYKTTPPVLDSDGSFFLVSKLTVDKSRWQQGN
VFSCSVMHEALHNHYTQKSLSLSPGK
SEQ ID NO: 62
(006/LC; T164C is underlined)
DIQMTQSPSSLSASVGDRVTITCRASQGISNYLAWYQQKTGKVPKFLIYEASTLQ
SGVPSRFSGGGSGTDFTLTISSLQPEDVATYYCQNYNSAPFTFGPGTKVDIKRTVA
APSVFIFPPSDEQLKSGTASVVCLLNNFYPREAKVQWKVDNALQSGNSQESVCE
QDSKDSTYSLSSTLTLSKADYEKHKVYACEVTHQGLSSPVTKSFNRGEA
SEQ ID NO: 63
(J591/HC)
EVQLVQSGPEVKKPGATVKISCKTSGYTFTEYTIHWVKQAPGKGLEWIGNINPN
NGGTTYNQKFEDKATLTVDKSTDTAYMELSSLRSEDTAVYYCAAGWNFDYWG
QGTLLTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVS
NKALPAPIEKTISKAKGQPREPQVYTLPPSREEMTKNQVSLWCLVKGFYPSDIAV
EWESNGQPENNYKTTPPVLDSDGSFFLYSKLTVDKSRWQQGNVFSCSVMHEAL
HNHYTQKSLSLSPGK
SEQ ID NO: 64
(J591/LC)
DIQMTQSPSSLSTSVGDRVTLTCKASQDVGTAVDWYQQKPGPSPKLLIYWASTR
HTGIPSRFSGSGSGTDFTLTISSLQPEDFADYYCQQYNSYPLTFGPGTKVDIKRTV
AAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
SEQ ID NO: 65
(006/HC; P171C is underlined)
QVQLVESGGGVVQPGRSLRLSCAASGFAFSRYGMHWVRQAPGKGLEWVAVIW
YDGSNKYYADSVKGRFTISRDNSKNTQYLQMNSLRAEDTAVYYCARGGDFLYY
YYYGMDVWGQGTTVTVSSASTKGPSVFPLAPSSKSTSGGTAALGCLVKDYFPE
PVTVSWNSGALTSGVHTFCAVLQSSGLYSLSSVVTVPSSSLGTQTYICNVNHKPS
NTKVDKKVEPKSADKTHTCPPCPAPELLGGPSVFLFPPKPKDTLMISRTPEVTCV
VVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLSC
AVKGFYPSDIAVEWESNGQPENNYKTTPPVLDSDGSFFLVSKLTVDKSRWQQGN
VFSCSVMHEALHNHYTQKSLSLSPGK
SEQ ID NO: 66
(006/LC; S165C is underlined)
DIQMTQSPSSLSASVGDRVTITCRASQGISNYLAWYQQKTGKVPKFLIYEASTLQ
SGVPSRFSGGGSGTDFTLTISSLQPEDVATYYCQNYNSAPFTFGPGTKVDIKGQP
KAAPSVTLFPPSSEELQANKATLVCLISDFYPGAVTVAWKADSSPVKAGVETTTP
CKQSNNKYAASSYLSLTPEQWKSHRSYSCQVTHEGSTVEKTVAPTEAS

### Example 8

### Expression, Purification and Characterization of FAP×CD40 Bispecific Antibodies

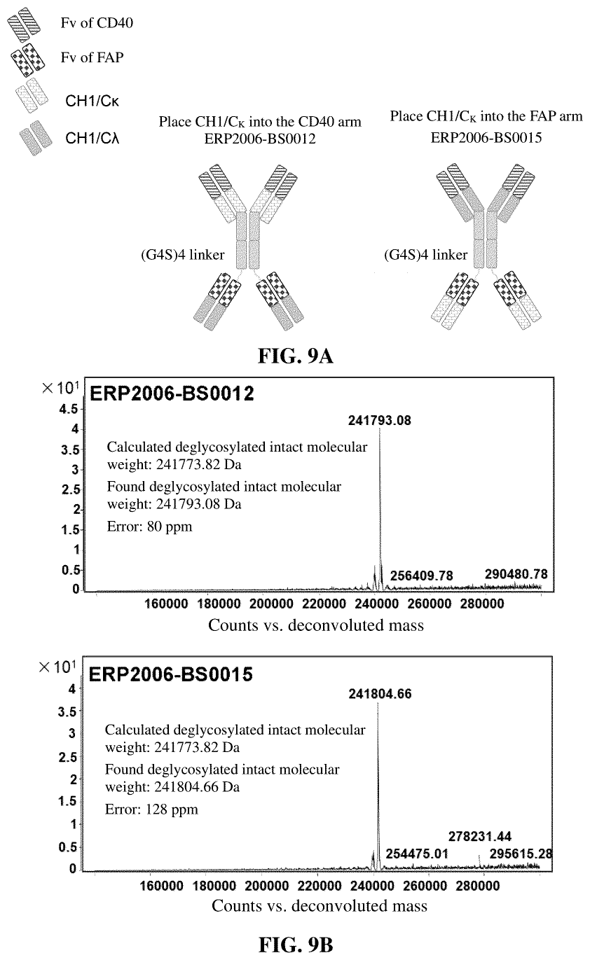

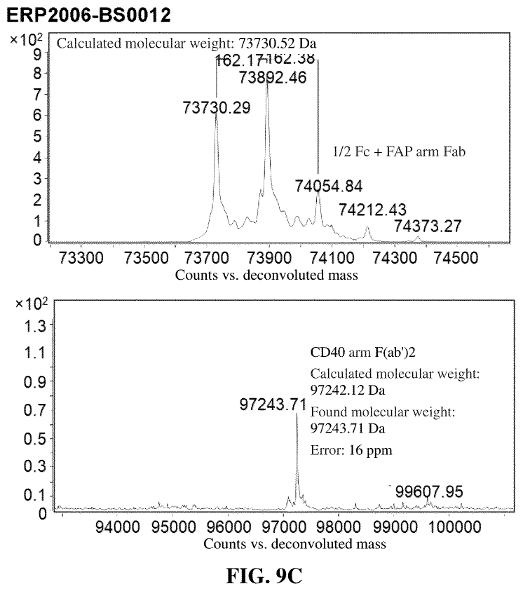

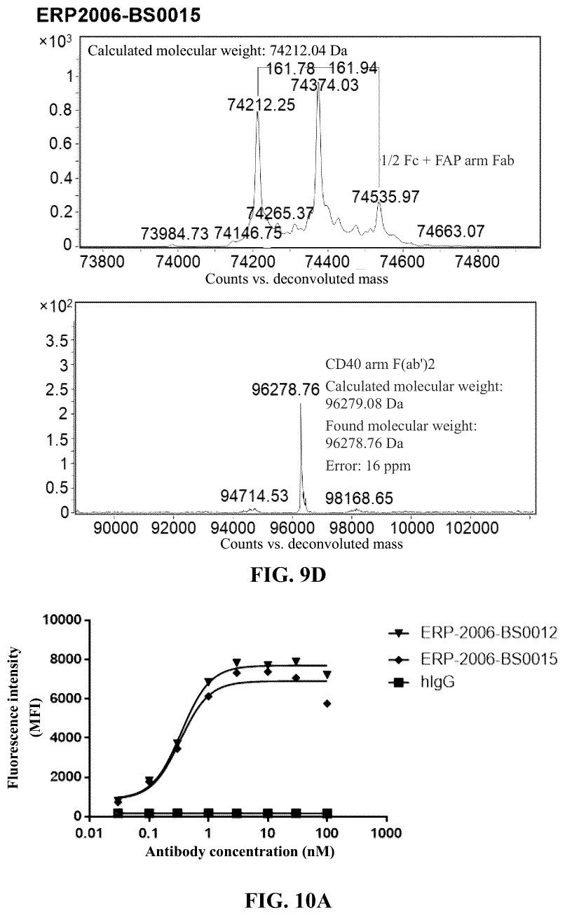

FAP×CD40 bispecific antibodies were constructed and expressed according to the antibody sequences and plasmid ratios shown in Table 12 (see FIG. 9A for molecular formats).

TABLE 12
The plasmid ratios of FAP × CD40 bispecific
antibodies comprising non-natural disulfide bonds

| Protein No. (heavy | | | |
|---|---|---|---|
| chain:CD40 arm | | | |
| light chain:FAP | | | |
| arm light chain | | CD40 arm | FAP arm |
| plasmid molar ratio) | Heavy chain | light chain | light chain |
| ERP2006-BS0012 | SEQ ID | SEQ ID | SEQ ID |
| (2:3:3) | NO: 67 | NO: 68 | NO: 69 |
| ERP2006-BS0015 | SEQ ID | SEQ ID | SEQ ID |
| (2:3:3) | NO: 70 | NO: 71 | NO: 72 |

SEQ ID NO: 67
(CD40-Fc-FAP/HC; P171C is underlined)
QVQLVQSGAEVKKPGASVKVSCKASGYILTTYWITWVRQAPGQGLEWMGDIH
PGSGSTKYNEKFKSRVTLTVDTSISTAYMELSRLRSEDTAVYYCARRDYWGQGT
TVTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSC
DKTHTCPPCPAPEFEGGPSVFLFPPKPKDTLMISRTPEVTCVVVDVSHEDPEVKF
NWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHN
HYTQKSLSLSPGGGGGSGGGGSGGGGSGGGGSQVQLQQSGVEVKKPGASVTV
SCRASGYSFADHFIHWVRQAPGQGFQWMGWINPNRGVTHHAQDFQGRVAMTR
DMSTDTVYMELTSLRSDDTAVYYCARDASLTARPYYFYGFDVWGQGTLVTVSS
ASTKGPSVFPLAPSSKSTSGGTAALGCLVKDYFPEPVTVSWNSGALTSGVHTFCA
VLQSSGLYSLSSVVTVPSSSLGTQTYICNVNHKPSNTKVDKKVEPKSS
SEQ ID NO: 68
(CD40/LC)
DIVMTQSPLSLPVTPGEPASISCRSSQNIVNSQGNTYLEWYLQKPGQSPQLLIYK
VTNRFSGVPDRFSGSGSGTDFTLKISRVEAEDVGVYYCFQASLVPWTFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
SEQ ID NO: 69
(FAP/LC; S165C is underlined)
DIQMTQSPSSVSASVGDRVTITCRASQGISSWLAWYQQKPGKAPKLLIYAASSLQ
SGVPSRFSGSGSGTDFTLTISSLQPEDFATYYCQQANSFPPAFGQGTKVEIKGQPK
AAPSVTLFPPSSEELQANKATLVCLISDFYPGAVTVAWKADSSPVKAGVETTTPC
KQSNNKYAASSYLSLTPEQWKSHRSYSCQVTHEGSTVEKTVAPTESS
SEQ ID NO: 70
(CD40-Fc-FAP/HC; P171C is underlined)
QVQLVQSGAEVKKPGASVKVSCKASGYILTTYWITWVRQAPGQGLEWMGDIH
PGSGSTKYNEKFKSRVTLTVDTSISTAYMELSRLRSEDTAVYYCARRDYWGQGT
TVTVSSASTKGPSVFPLAPSSKSTSGGTAALGCLVKDYFPEPVTVSWNSGALTSG
VHTFCAVLQSSGLYSLSSVVTVPSSSLGTQTYICNVNHKPSNTKVDKKVEPKSSD
KTHTCPPCPAPEFEGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHY
TQKSLSLSPGGGGGSGGGGSGGGGSGGGGSQVQLQQSGVEVKKPGASVTVSC
RASGYSFADHFIHWVRQAPGQGFQWMGWINPNRGVTHHAQDFQGRVAMTRD
MSTDTVYMELTSLRSDDTAVYYCARDASLTARPYYFYGFDVWGQGTLVTVSSA
STKGPSVFPLAPSSKSTSGGTAALGCLVKDYFPEPVTVSWNSGALTSGVHTCPAV
LQSSGLYSLSSVVTVPSSSLGTQTYICNVNHKPSNTKVDKKVEPKSC
SEQ ID NO: 71
(CD40/LC; S165C is underlined)
DIVMTQSPLSLPVTPGEPASISCRSSQNIVNSQGNTYLEWYLQKPGQSPQLLIYK
VTNRFSGVPDRFSGSGSGTDFTLKISRVEAEDVGVYYCFQASLVPWTFGGGTKV
EIKGQPKAAPSVTLFPPSSEELQANKATLVCLISDFYPGAVTVAWKADSSPVKAG
VETTTPCKQSNNKYAASSYLSLTPEQWKSHRSYSCQVTHEGSTVEKTVAPTESS
SEQ ID NO: 72
(FAP/LC)
DIQMTQSPSSVSASVGDRVTITCRASQGISSWLAWYQQKPGKAPKLLIYAASSLQ
SGVPSRFSGSGSGTDFTLTISSLQPEDFATYYCQQANSFPPAFGQGTKVEIKRTVA
APSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC

TABLE 13
The CDR sequences of the parent CD40 antibody 9E5-
25 and the parent FAP antibody Ab10

| Name of antibody | CDR | Sequence | SEQ ID NO: |
|---|---|---|---|
| 9E5-25 | HCDR1 | TYWIT | 73 |
| | HCDR2 | DIHPGSGSTKYNEKFKS | 74 |
| | HCDR3 | RDY | 1 |
| | LCDR1 | RSSQNIVNSQGNTYLE | 75 |
| | LCDR2 | KVTNRFS | 76 |
| | LCDR3 | FQASLVPWT | 77 |
| Ab10 | HCDR1 | DHFIH | 80 |
| | HCDR2 | WINPNRGVTHHAQDFQG | 81 |
| | HCDR3 | DASLTARPYYFYGFDV | 82 |
| | LCDR1 | RASQGISSWLA | 83 |
| | LCDR2 | AASSLQS | 84 |
| | LCDR3 | QQANSFPPA | 85 |

SEQ ID NO: 78
(CD40/VH)
QVQLVQSGAEVKKPGASVKVSCKASGYILTTYWITWVRQAPGQGLEWMGD
IHPGSGSTKYNEKFKSRVTLTVDTSISTAYMELSRLRSEDTAVYYCARRD
YWGQGTTVTVSS
SEQ ID NO: 79
(CD40/VL)
DIVMTQSPLSLPVTPGEPASISCRSSQNIVNSQGNTYLEWYLQKPGQSPQ
LLIYKVTNRFSGVPDRFSGSGSGTDFTLKISRVEAEDVGVYYCFQASLVP
WTFGGGTKVEIK
SEQ ID NO: 86
(FAP/VH)
QVQLQQSGVEVKKPGASVTVSCRASGYSFADHFIHWVRQAPGQGFQWMGW
INPNRGVTHHAQDFQGRVAMTRDMSTDTVYMELTSLRSDDTAVYYCARDA
SLTARPYYFYGFDVWGQGTLVTVSS
SEQ ID NO: 87
(FAP/VL)
DIQMTQSPSSVSASVGDRVTITCRASQGISSWLAWYQQKPGKAPKLLIYA
ASSLQSGVPSRFSGSGSGTDFTLTISSLQPEDFATYYCQQANSFPPAFGQ
GTKVEIK

The SEC purity of the bispecific antibodies after one-step ProteinA purification was >99%, the deglycosylated intact molecular weight, the reduced molecular weight and the molecular weight after IdeS digestion all met expectations, and no unassignable byproducts were found (FIG. 9B and FIG. 9C).

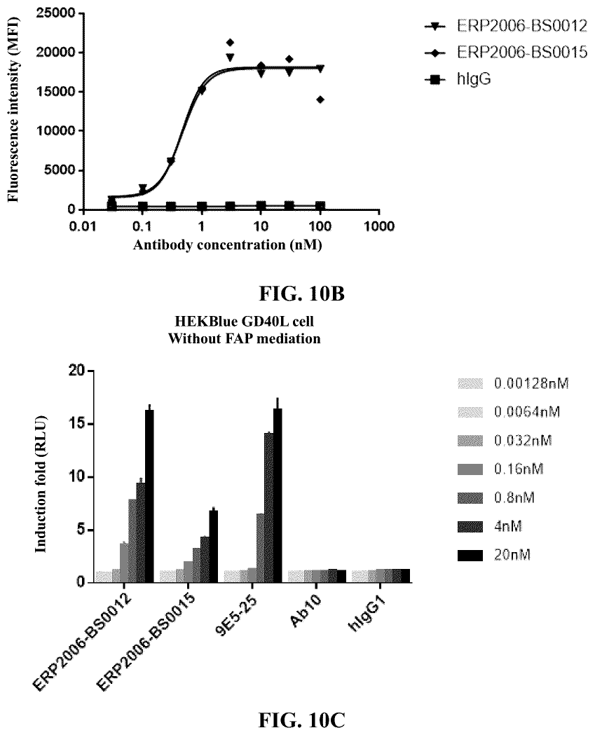

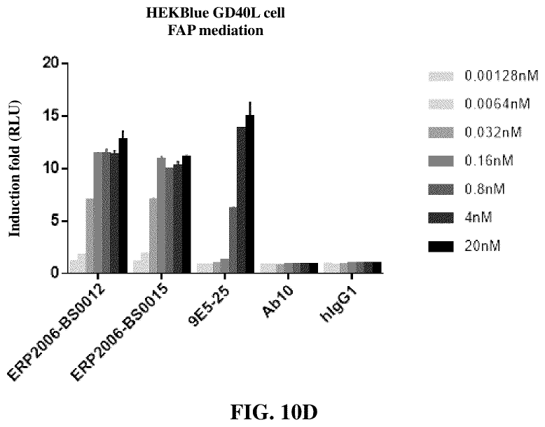

As shown in FIG. 10A and FIG. 10B, the FACS binding EC 50 values of the bispecific antibodies ERP2006-BS0012 and ERP2006-BS0015 to CD40 were 0.471 nM and 0.456 nM, respectively. The FACS binding EC 50 values of ERP2006-BS0012 and ERP2006-BS0015 to FAP were 0.349 nM and 0.336 nM, respectively. The bispecific antibodies ERP2006-BS0012 and ERP2006-BS0015 have similar affinity for FAP to the parent FAP monoclonal antibody AblO and similar affinity for CD40 to the parent CD40 monoclonal antibody 9E5-25.

FIG. 10C and FIG. 10D show that ERP2006-BS0012 and ERP2006-BS0015 have CD40 activation activity in the absence of FAP, but the activity is weaker than that of the parent antibody 9E5-25; this characteristic leads to a reduction in the peripheral tissue CD40 activation activity of the bispecific antibodies in the absence of FAP, and can reduce the peripheral toxicity of the CD40 monoclonal antibody. In the presence of FAP protein, the CD40 activation activity of ERP2006-BS0012 and ERP2006-BS0015 was significantly enhanced, which indicates that their CD40 activation activity is FAP-dependent. Their CD40 activation activity is stronger than that of the parent CD40 monoclonal antibody 9E5-25; this characteristic makes the bispecific antibodies have stronger CD40 activation activity in tumors highly expressing FAP.

Although the foregoing invention has been described in detail by way of drawings and examples for purposes of clarity of understanding, the description and examples should not be construed as limiting the scope of the present disclosure. The disclosures of all patents and scientific literature cited herein are clearly incorporated by reference in their entirety.

1. A dimerized polypeptide comprising a heavy chain constant region 1 (CH1) and a light chain constant region (CL), wherein: CH1 and CL comprise natural-non-cysteine-to-cysteine amino acid substitutions at positions selected from one or more of (i-1) to (i-6):
(i-1) position 170 of CH1 and position 164 of CL,
(i-2) position 128 of CH1 and position 121 of CL,
(i-3) position 129 of CH1 and position 121 of CL,
(i-4) position 131 of CH1 and position 119 of CL,
(i-5) position 141 of CH1 and position 135 of CL, and
(i-6) position 171 of CH1 and position 165 of CL.

2. The dimerized polypeptide according to claim 1, wherein the CH1 further comprises a natural-cysteine-to-non-cysteine amino acid substitution, and the CL further comprises a natural-cysteine-to-non-cysteine amino acid substitution.

3. The dimerized polypeptide according to claim 1, wherein: the CH1 and CL comprise the following amino acid substitutions:
(a) C220A in CH1 and C214A in CL; and
(b) amino acid substitutions selected from at least one of the following:
(b-1) F170C in CH1 and T164C in CL;
(b-2) L128C in CH1 and S121C in CL;
(b-3) A129C in CH1 and S121C in CL;
(b-4) S131C in CH1 and P119C in CL;
(b-5) A141C in CH1 and L135C in CL; and
(b-6) P171C in CH1 and S165C in CL.

4. The dimerized polypeptide according to claim 1, wherein: the CH1 and CL further comprise amino acid substitutions that cause an electrostatic interaction interface to be formed between CH1 and CL.

5. The dimerized polypeptide according to claim 1, wherein: the CH1 and CL comprise amino acid substitutions selected from any one of (1)-(4):
(1) C220A in CH1 and C214A in CL; F170C in CH1 and T164C in CL; and T139R in CH1 and S114E in CL;
(2) C220A in CH1 and C214A in CL; F170C in CH1 and T164C in CL; and T139D in CH1 and S114K in CL;
(3) C220A in CH1 and C214A in CL; P171C in CH1 and S165C in CL; and T139R in CH1 and S114E in CL; and
(4) C220A in CH1 and C214A in CL; P171C in CH1 and S165C in CL; and T139D in CH1 and S114K in CL.

6. An antigen-binding protein comprising the dimerized polypeptide according to claim 1, wherein the antigen-binding protein comprises or is a bispecific or multispecific antibody.

7. The antigen-binding protein according to claim 6, comprising a first antigen-binding domain, wherein the first antigen-binding domain comprises a Fab comprising a first heavy chain variable region VH1, a first light chain variable region VL1; or
comprising a first antigen-binding domain and a second antigen-binding domain, wherein the wherein the first antigen-binding domain comprises a Fab comprising a first heavy chain variable region VH1, a first light chain variable region VL1, a first CH1 and a first CL, the second antigen-binding domain comprises a second heavy chain variable region VH2 and a second light chain variable region VL2, a second CH1 and a second CL, and the first antigen-binding domain and the second antigen-binding domain bind to different antigens or bind to different epitopes on the same antigen.

8. The antigen-binding protein according to claim 7, wherein: the first CH1 and the first CL comprise the following amino acid substitutions:
(a) C220A in CH1 and C214A in CL; and
(b) amino acid substitutions selected from at least one of the following:
(b-1) F170C in CH1 and T164C in CL;
(b-2) L128C in CH1 and S121C in CL;
(b-3) A129C in CH1 and S121C in CL;
(b-4) S131C in CH1 and P119C in CL;
(b-5) A141C in CH1 and L135C in CL; and
(b-6) P171C in CH1 and S165C in CL.

9. The antigen-binding protein according to claim 74, wherein:
the first CH1 and the first CL further comprise amino acid substitutions that cause an electrostatic interaction interface to be formed between the first CH1 and the first CL, and wherein the amino acid substitutions are at position 139 of the first CH1 and position 114 of the first CL; and/or the second CH1 and the second CL comprise amino acid substitutions that cause an electrostatic interaction interface to be formed between the second CH1 and the second CL, and wherein the amino acid substitutions are at position 139 of the second CH1 and position 114 of the second CL.

10. The antigen-binding protein according to claim 7, wherein:
the first CH1 and the first CL comprise the following amino acid substitutions:
(a) C220A in CH1 and C214A in CL;
(b) amino acid substitutions selected from at least one of the following:
(b-1) F170C in CH1 and T164C in CL;
(b-2) L128C in CH1 and S121C in CL;
(b-3) A129C in CH1 and S121C in CL;
(b-4) S131C in CH1 and P119C in CL;
(b-5) A141C in CH1 and L135C in CL; and
(b-6) P171C in CH1 and S165C in CL; and
(c) amino acid substitutions selected from any one of the following:
(c-1) T139R in CH1 and S114E in CL;
(c-2) T139R in CH1 and S114D in CL;
(c-3) T139K in CH1 and S114E in CL; and
(c-4) T139K in CH1 and S114D in CL;
and the second CH1 and the second CL comprise amino acid substitutions selected from any one of the following:
(1) T139D in CH1 and S114K in CL;
(2) T139D in CH1 and S114R in CL;
(3) T139E in CH1 and S114K in CL; and
(4) T139E in CH1 and S114R in CL;
or,
the first CH1 and the first CL comprise the following amino acid substitutions:
(a) C220A in CH1 and C214A in CL;
(b) amino acid substitutions selected from at least one of the following:
(b-1) F170C in CH1 and T164C in CL;
(b-2) L128C in CH1 and S121C in CL;
(b-3) A129C in CH1 and S121C in CL;
(b-4) S131C in CH1 and P119C in CL;
(b-5) A141C in CH1 and L135C in CL; and
(b-6) P171C in CH1 and S165C in CL; and
(c) amino acid substitutions selected from any one of the following:
(c-1) T139D in CH1 and S114K in CL;
(c-2) T139D in CH1 and S114R in CL;
(c-3) T139E in CH1 and S114K in CL; and
(c-4) T139E in CH1 and S114R in CL;
and the second CH1 and the second CL comprise amino acid substitutions selected from any one of the following:
(1) T139R in CH1 and S114E in CL;
(2) T139R in CH1 and S114D in CL;
(3) T139K in CH1 and S114E in CL; and
(4) T139K in CH1 and S114D in CL.

11. The antigen-binding protein according to any one of claims claim 740, wherein:
(1) the first CH1 and the first CL comprise the following amino acid substitutions:
(a) C220A in CH1 and C214A in CL;
(b) F170C in CH1 and T164C in CL; and
(c) T139R in CH1 and S114E in CL;
and the second CH1 and the second CL comprise the following amino acid substitutions:
T139D in CH1 and S114K in CL;
(2) the first CH1 and the first CL comprise the following amino acid substitutions:
(a) C220A in CH1 and C214A in CL;
(b) F170C in CH1 and T164C in CL; and
(c) T139D in CH1 and S114K in CL;
and the second CH1 and the second CL comprise the following amino acid substitutions:
T139R in CH1 and S114E in CL;
(3) the first CH1 and the first CL comprise the following amino acid substitutions:
(a) C220A in CH1 and C214A in CL;
(b) P171C in CH1 and S165C in CL; and
(c) T139R in CH1 and S114E in CL;
and the second CH1 and the second CL comprise the following amino acid substitutions:
T139D in CH1 and S114K in CL; or
(4) the first CH1 and the first CL comprise the following amino acid substitutions:
(a) C220A in CH1 and C214A in CL;
(b) P171C in CH1 and S165C in CL; and
(c) T139D in CH1 and S114K in CL;
and the second CH1 and the second CL comprise the following amino acid substitutions:
T139R in CH1 and S114E in CL.

12. The antigen-binding protein according to claim 7, wherein: the first CL is from an antibody κ light chain (Cκ); the second CL is from an antibody λ light chain (Cλ) or κ light chain (Cκ).

13. The antigen-binding protein according to claim 6, wherein: the antigen-binding protein further comprises a Fc region comprising a first subunit Fc1 and a second subunit Fc2 capable of associating with each other, and the Fc1 and/or the Fc2 are/is selected from the group consisting of Fc of human IgG1, IgG2, IgG3 and IgG4.

14. The antigen-binding protein according to claim 13, wherein: the Fc 1 and/or the Fc2 comprise(s) a modification that alters the half-life of the antigen-binding protein, wherein the half-life is dependent on FcRn binding affinity; the Fc1 and/or the Fc2 comprise(s) a modification that alters effector functions, wherein binding affinity for Fcγ receptors or C1q complement protein is increased or decreased; and/or the Fc1 and Fc2 comprise such amino acid substitutions that Fc1 is preferentially paired with Fc2 over Fc1.

15. The antigen-binding protein according to claim 7, wherein the first antigen-binding domain specifically binds to CTLA-4, and the second antigen-binding domain specifically binds to PD-1; or, the first antigen-binding domain specifically binds to PD-1, and the second antigen-binding domain specifically binds to CTLA-4.

16. A nucleic acid molecule encoding the dimerized polypeptide according to claim 1.

17. A host cell comprising the nucleic acid molecule according to claim 16.

18. A method for preparing the dimerized polypeptide according to claim 1, comprising the following steps:
(1) transforming a host cell with a nucleic acid expression vector comprising the nucleic acid molecule according to claim 16;
(2) culturing the host cell under conditions that permit synthesis of the antigen-binding protein to obtain a cell culture; and
(3) recovering the antigen-binding protein from the cell culture.

19. A pharmaceutical composition comprising the antigen-binding protein according to claim 6 and a pharmaceutically acceptable carrier.

20. A method of treating a cancer, an autoimmune disease or an inflammatory disease in a subject in need thereof, the method comprising: administering a therapeutically effective amount of the antigen-binding protein according to claim 6 to the subject.

21. The dimerized polypeptide according to claim 4, wherein the amino acid substitutions that cause an electrostatic interaction interface to be formed between CH1 and CL are at position 139 of CH1 and position 114 of CL.
